(19) United States
(12) Patent Application Publication
(10) Pub. No.: US 2023/0389844 A1
(43) Pub. Date: Dec. 7, 2023

(54) SENSING SYSTEMS AND METHODS FOR DIAGNOSING KIDNEY DISEASE

(71) Applicant: Dexcom, Inc., San Diego, CA (US)

(72) Inventors: Matthew L. JOHNSON, San Diego, CA (US); Qi AN, San Diego, CA (US); Rush BARTLETT, San Diego, CA (US); John PADERI, San Diego, CA (US)

(21) Appl. No.: 18/327,012

(22) Filed: May 31, 2023

Related U.S. Application Data
(60) Provisional application No. 63/365,702, filed on Jun. 1, 2022.
(60) Provisional application No. 63/376,673, filed on Sep. 22, 2022.
(60) Provisional application No. 63/387,078, filed on Dec. 12, 2022.
(60) Provisional application No. 63/377,332, filed on Sep. 27, 2022.
(60) Provisional application No. 63/403,568, filed on Sep. 2, 2022.
(60) Provisional application No. 63/403,582, filed on Sep. 2, 2022.

(51) Int. Cl. A61B 5/20; A61B 5/1486; A61B 5/00; A61B 5/145; A61B 5/0205; A61B 5/07; G16H 40/67; G16H 50/20

(52) U.S. Cl. CPC A61B 5/201; A61B 5/14865; A61B 5/7275; A61B 5/4842; A61B 5/486; A61B 5/746; A61B 5/14532; A61B 5/0205; A61B 5/076; A61B 5/7282; A61B 5/7267; G16H 40/67; G16H 50/20

(57) ABSTRACT

Certain aspects of the present disclosure provide a monitoring system comprising a continuous analyte sensor configured to generate analyte measurements associated with analyte levels of a patient, and a sensor electronics module coupled to the continuous analyte sensor and configured to receive and process the analyte measurements.

### CROSS-REFERENCE TO RELATED APPLICATIONS

This application claims priority to and benefit of U.S. Provisional Application No. 63/365,702, filed Jun. 1, 2022, and U.S. Provisional Application No. 63/376,673, filed Sep. 22, 2022, and U.S. Provisional Application No. 63/387,078, filed Dec. 12, 2022, and U.S. Provisional Application No. 63/377,332, filed Sep. 27, 2022, and U.S. Provisional Application No. 63/403,568, filed Sep. 2, 2022, and U.S. Provisional Application No. 63/403,582, filed Sep. 2, 2022, which are hereby assigned to the assignee hereof and hereby expressly incorporated by reference in their entirety as if fully set forth below and for all applicable purposes.

### BACKGROUND

The kidneys are responsible for many critical functions within the human body including, but not limited to, filtering waste and excess fluids, which are excreted in the urine, and removing acid that is produced by the cells of the body to maintain a healthy balance of water, salts, and minerals (e.g., such as sodium, calcium, phosphorus, and potassium) in the blood. Thus, the kidneys play a major role in homeostasis by renal mechanisms that transport and regulate water, salt, and mineral secretion, reabsorption, and excretion. Further, kidneys secrete renin (e.g., angiotensinogenase), which forms part of the renin-angiotensin-aldosterone system (RAAS) that mediates extracellular fluid and arterial vasoconstriction (e.g., blood pressure). More specifically, high blood pressure (e.g., hypertension) can be regulated through RAAS inhibitors such as angiotensin-converting enzyme (ACE) inhibitors and angiotensin receptor blockers (ARBs). Should the kidney become diseased or injured, the impairment or loss of these functions can cause significant damage to the human body.

Kidney disease occurs when a kidney becomes diseased or injured. Kidney disease is generally classified as either acute or chronic based upon the duration of the disease. Acute kidney injury (AKI) (also referred to as “acute kidney failure” or “acute renal failure”) is usually caused by a sudden event that leads to kidney malfunction, such as dehydration, blood loss from major surgery or injury, and/or the use of medicines. On the other hand, chronic kidney disease (CKD) (or “chronic kidney failure”) is usually caused by a long-term disease, such as high blood pressure or diabetes, which slowly damages the kidneys and reduces their function over time.

As briefly mentioned above, the kidneys play a major role in potassium homeostasis by renal mechanisms that transport and regulate water, salt, and mineral secretion, reabsorption, and excretion. In some cases, elevated potassium levels in a patient with untreated CKD may lead to hyperkalemia. Hyperkalemia is the medical term that describes a potassium level in the blood that is higher than normal (e.g., higher than normal blood potassium levels between 3.6 and 5.2 millimoles per liter (mmol/L)). Hyperkalemia can increase the risk of cardiac arrhythmia episodes and even sudden death. Symptoms associated with mild hyperkalemia include muscle weakness, numbness, tingling, nausea, or other unusual feelings, while symptoms of very elevated potassium levels include heart palpitations, shortness of breath, chest pain, nausea, or vomiting. In more severe cases of hyperkalemia, patients may experience respiratory failure, sudden cardiac death, or other mortality-driven events.

Similarly, low potassium levels in a patient with untreated CKD may lead to the progression of hypokalemia. Hypokalemia is the medical term that describes potassium levels in the blood that are lower than normal. Patients with CKD may develop hypokalemia due to gastrointestinal potassium loss from, e.g., diarrhea or vomiting or renal potassium loss from non-potassium-sparing diuretics (e.g., diuretics used to increase the amount of fluid passed from the body in urine, without regard for the amount of potassium being lost from the body in the urine). Similar with hyperkalemia, severe hypokalemia can lead to symptoms of respiratory failure, sudden cardiac death, or other mortality-driven events.

A lack of readily noticeable symptoms in many patients is why hyperkalemia and hypokalemia are often referred to as “silent killers,” especially when patients have become sensitized to irregular potassium levels. For example, in severe cases where hypokalemia or hyperkalemia leads to severe symptoms such as mortality-driven events, the diagnosis, being hypokalemia or hyperkalemia, as the mediating mechanism, may not be readily apparent by the time a patient is evaluated by medical personnel.

CKD may also alter glucose homeostasis of a patient, thereby making CKD an independent risk factor for hypoglycemia. In particular, the kidneys also play an important role in the regulation of blood glucose (e.g., blood sugar). With respect to renal involvement in glucose homeostasis, the primary mechanisms include release of glucose into circulation via gluconeogenesis, uptake of glucose from the circulation to satisfy the kidneys' energy needs, and reabsorption of glucose at the level of the proximal tubule of the kidney. For example, gluconeogenesis is the formation of glucose from precursors (e.g., lactate, glycerol and/or amino acids). During renal gluconeogenesis, glucose is formed by the kidneys and released into circulation. Gluconeogenesis, primarily in the liver but also in the kidney, occurs to maintain glucose homeostasis by preventing low blood glucose.

As kidney function declines, however, the formation of glucose also declines, and thus limits the kidney's ability to react to falling blood glucose. In some cases, a reduction in the kidney's ability to react to falling blood glucose levels may lead to hypoglycemia. Hypoglycemia is the medical term that describes a blood sugar (e.g., glucose) level that is lower than normal (e.g., a blood sugar level below 70 milligrams per deciliter (mg/dL), or 3.9 millimoles per liter (mmol/L)). 1.7% of hospitalizations annually are due to hypoglycemia for early-stage CKD (CKD<3). Further, for end stage renal disease (ESRD)-related hospitalizations, 3.6% are due to hypoglycemia with a 30% mortality rate. In particular, severe hypoglycemia can lead to damage of the heart muscle, neurocognitive dysfunction, and in certain cases, seizures or even death.

As such, kidney dysfunction may result in lower and/or longer low glucose levels. Impaired kidneys may be slower, and, in some cases, less effective, at combating falling glucose levels resulting in poor glucose control (e.g., given kidneys filter insulin, reabsorb glucose filtered from the proximal tubule, and generate glucose through gluconeogenesis). Hypoglycemic events in kidney disease result from decreased insulin clearance and impaired gluconeogenesis by the kidneys. For ESRD-related hospitalizations, dialysis is also a compounding factor.

Further, decreased insulin metaboiism and clearance may occur as a result of declining kidney health. Insulin is a hormone that allows the body to use glucose for energy, or store glucose as fat. In other words, insulin stimulates potassium and glucose uptake by a patient's cells, reducing serum (e.g., extracellular) potassium and glucose levels. Insulin is cleared by the kidneys; thus, as kidney function declines, insulin is cleared more slowly. Accordingly, a typical dose of insulin may have a prolonged and/or pronounced effect on glucose in a patient with kidney dysfunction. As kidney dysfunction progresses, insulin may have an even more prolonged and/or pronounced effect on glucose. Thus, a patient with kidney disease may be at risk for hypoglycemia, and the risk increases with disease progression. For example, a patient with CKD may find that an insulin dose that is predictive of glucose clearance at one point in time later results in hypoglycemia.

Kidney dysfunction may also result in higher and/or longer high glucose levels. For example, impaired kidneys may be slower and, in some cases, less effective at reducing glucose levels (i.e., clearing glucose) given kidneys are response for filtering, reabsorbing, and consuming glucose from the blood. Patients suffering from higher and/or longer high glucose levels may be diagnosed as hyperglycemic. Hyperglycemia is the medical term that describes a blood sugar (e.g., glucose) level that is higher than normal (e.g., significantly elevated blood sugar levels, usually elevated above 180 to 200 mg/dL, or 10 to 11.1 mmol/L). Patients with hyperglycemia may suffer from an array of negative physiological effects (for example, nerve damage (neuropathy), kidney failure, skin ulcers, diabetic ketoacidosis, or bleeding into the vitreous of the eye) associated with the deterioration of small blood vessels.

Other health complications may also develop with CKD, including but not limited to anemia, bone weakness, fluid retention, gout, heart disease, hypertension, hyperphosphatemia, metabolic acidosis, uremia, etc. These complications, as well as those described above, may occur more frequently and with greater severity as kidney disease progresses, leading to poor quality of life and increased morbidity and mortality.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example decision support system that may be used in connection with implementing embodiments of the present disclosure.

FIG. 2 is a diagram conceptually illustrating an example continuous analyte monitoring system including example continuous analyte sensor(s) with sensor electronics, in accordance with certain aspects of the present disclosure.

FIG. 3 illustrates example inputs and example metrics that are calculated based on the inputs for use by the decision support system of FIG. 1, according to some embodiments disclosed herein.

FIG. 4 is an example workflow for generating a kidney disease prediction for a patient utilizing a continuous analyte sensor and providing one or more recommendations for treatment based on the generated kidney disease prediction, according to certain embodiments of the present disclosure.

FIG. 5 is a flow diagram depicting a method for training machine learning models to provide predictions associated with kidney disease, according to certain embodiments of the present disclosure.

FIG. 6 is a block diagram depicting a computing device configured to perform the operations of FIGS. 4 and/or 5, according to certain embodiments disclosed herein.

FIGS. 7A-7B depict exemplary enzyme domain configurations for a continuous multi-analyte sensor, according to certain embodiments disclosed herein.

FIGS. 7C-7D depict exemplary enzyme domain configurations for a continuous multi-analyte sensor, according to certain embodiments disclosed herein.

FIG. 7E depicts an exemplary enzyme domain configuration for a continuous multi-analyte sensor, according to certain embodiments disclosed herein.

FIGS. 8A-8B depict alternative views of an exemplary dual electrode enzyme domain configuration for a continuous multi-analyte sensor, according to certain embodiments disclosed herein.

FIGS. 8C-8D depict alternative views of an exemplary dual electrode enzyme domain configuration for a continuous multi-analyte sensor, according to certain embodiments disclosed herein.

FIG. 8E depicts an exemplary dual electrode configuration for a continuous multi-analyte sensor, according to certain embodiments disclosed herein.

FIG. 9A depicts an exemplary enzyme domain configuration for a continuous multi-analyte sensor, according to certain embodiments disclosed herein.

FIGS. 9B-9C depict alternative exemplary enzyme domain configurations for a continuous multi-analyte sensor, according to certain embodiments disclosed herein.

FIG. 10 depicts an exemplary enzyme domain configuration for a continuous multi-analyte sensor, according to certain embodiments disclosed herein.

FIGS. 11A-11D depict alternative views of exemplary dual electrode enzyme domain configurations G1-G4 for a continuous multi-analyte sensor, according to certain embodiments disclosed herein.

FIGS. 12A-12B schematically illustrate an example configuration and component of a device for measuring an electrophysiological signal and/or concentration of a target ion in a biological fluid in vivo, according to certain embodiments disclosed herein.

FIG. 13 schematically illustrates additional example configurations and component of a device for measuring an electrophysiological signal and/or a concentration of a target ion in a biological fluid in vivo, according to certain embodiments disclosed herein.

FIGS. 14A-14C schematically illustrate example configurations and components of a device for measuring an electrophysiological signal and/or concentration of a target analyte in a biological fluid in vivo, according to certain embodiments disclosed herein.

FIG. 15 is a diagram depicting an example continuous analyte monitoring system configured to measure target ions and/or other analytes as discussed herein, according to certain embodiments disclosed herein.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Accurate assessment of kidney function (as well as heart function, in some cases) is important as a screening tool and for monitoring disease progression and guiding prognosis at least with respect to chronic kidney disease (CKD). However, conventional disease diagnostic methods and systems for such diseases, including, but not limited to, glomerular filtration rate (GFR) tests, albumin-to-creatinine ratio (ACR) tests, electrocardiogram (ECG) monitoring, and blood tests for monitoring potassium levels of a patient, face many challenges with respect to accuracy and reliability. Further, conventional disease diagnostic methods and systems generally fail to provide an efficient and complete analysis of factors which may likely contribute to CKD, and thus, CKD is difficult to diagnose in its early stages.

The current standard for diagnosis of CKD is based on glomerular filtration rate (GFR). GFR is an assessment of the flow rate of filtered fluid through the kidney, and is determined via measurement or estimation of clearance rates of exogenous or endogenous filtration markers that are cleared exclusively through glomerular filtration. A clearance rate of a marker is the volume of blood plasma that is cleared of the marker per unit time and is used to approximate the GFR. Thus, GFR is used as an approximate measurement of kidney function, and can help determine the presence and severity of CKD.

GFR can be assessed from urinary or plasma clearance measurements of exogenous filtration markers (e.g., measured GFR (mGFR)), or from measured serum levels of endogenous filtration markers using formulas (e.g., estimated GFR (eGFR)). Yet, both of these methods have limitations relating to both administration of such tests and the data gathered therefrom. For example, in an mGFR test, multiple blood (or urinary) tests are performed to calculate the clearance rate of the exogenous marker(s), such as iothalamate or inulin, over a period of a few hours (e.g., 5-6 hours). Samples are collected over several hours due to early phase rapid decay of the markers, which makes it impossible to determine an initial point of marker administration. Therefore, both the number of samples and the collection time period are greater to compensate for missing early data. As a result, mGFR tests may be costly, time-intensive, intrusive, and painful for patients. Additionally, mGFR tests require the patient to visit a clinic or other medical facility, thereby further adding to costly and inconvenient nature thereof.

Results from mGFR methods are also limited in assessment of kidney filtration and secretion. Renal reserve is the ability of individual nephrons of the kidneys to increase filtration by up to 30% in response to stress or high protein load. An otherwise healthy individual with full renal reserve capacity undergoing mGFR testing will have increased filtration due to their renal reserve. This can result in the overestimation of actual kidney function by mGFR testing. To normalize the mGFR results and account for renal reserve, renal reserve may be separately measured in a renal reserve test wherein an amino acid solution is administered to the patient and urine output thereafter monitored. However, for a CKD patient undergoing mGFR testing, renal reserve capacity may be impaired and there may only be a limited, if any, increase in filtration during testing, thereby creating uncertainty about how much, if at all, mGFR results have overestimated actual kidney function.

Additionally, because mGFR methods only determine overall kidney function, and do not differentiate between filtration activity and secretion activity, certain aspects of kidney health, such as reabsorption of glucose, cannot be determined through mGFR alone. Rather, additional testing may be required, such as for determining tubule health and the presence of tubulointerstitial fibrosis, which are both key aspects of kidney health. Thus, monitoring of additional kidney health markers, such as markers of tubule health, may be needed in addition to mGFR testing for more comprehensive assessments of global kidney health.

As an alternative to measuring GFR, estimating GFR provides a more convenient and rapid analysis for evaluating kidney function. Estimated GFR (eGFR) is typically determined based on an estimation of clearance rates of endogenous filtration markers such as creatinine, which can be determined from urine and/or blood samples from a patient. For example, blood samples may be collected from a patient over a 24 hour time period and creatinine levels therein measured for estimating the creatinine clearance rate of the patient based on several assumptions. Generally, different formulas may be used depending on whether the creatinine measurements were taken from blood or urine samples, and further depending on the patient's age, sex, weight, and/or ethnicity. However, the formulas for estimating GFR are based on certain assumptions and thus, such formulas may not be generalizable across all populations. Additionally, like mGFR, eGFR methods fail to take into consideration the serum potassium levels, as well as insulin levels, of the patient.

As another example, ACR tests are commonly used for screening and diagnosing kidney disease. In particular, an ACR test measures both albumin and creatinine in a one-time urine sample, also known as a spot urine sample. ACR is the first method of preference to detect elevated protein, specifically albumin, in the urine. Persistent increased albumin levels in the urine, i.e., increased albumin excretion, measured using the ACR test provides a marker of kidney damage; however, the test is not without flaws. For example, a failure to consider the influence of creatinine excretion on the ratio of albumin to creatinine in the current use of ACR tests may lead to inaccurate results, and consequently be misleading. Further, spot urine samples for ACR are more vulnerable to transient changes in excretion of creatinine and albumin than timed collections that average such changes over a longer time period; thus, random spot urine ACR results are less consistent than timed urine samples.

Both eGFR and ACR testing methods also suffer from their reliance on the measurement of creatinine levels. Creatinine is a waste product produced by the muscles from the breakdown of creatine, a non-protein compound which facilitates the recycling of adenosine triphosphate (ATP). Creatinine is then filtered out of the body by the kidney and released with urine. Because creatinine is produced by muscles, creatinine levels are a function of an individual's muscle mass and thus, in certain circumstances, may be more of a reflection of the patient's muscle mass rather than the patient's kidney function.

Creatinine levels are also subject to a biological delay of 24 to 48 hours. For example, in a patient suffering acute kidney injury (AKI), the patient's creatinine levels may not reflect the injury and thus, change in kidney function, until 24 to 48 hours after the injury. Thus, measured creatinine levels may not accurately reflect the patient's kidney function in real-time. In certain cases, by the time creatinine levels are elevated to a level where CKD may be diagnosed, approximate one-half or more of kidney function may be lost. In fact, kidney dysfunction is only accurately reflected in creatinine levels after significant GFR loss (e.g., <50 mg/dL), which corresponds to CKD stage 3 and beyond. ACR and eGFR are, therefore, not useful for diagnosis of chronic kidney disease until stage 3 or later.

Outside of GFR and ACR, another conventional method for identifying potassium imbalances and, therefore, a kidney problem, is electrocardiogram (ECG) monitoring. More particularly, ECG monitoring has been touted as a method used to recognize the arrhythmogenic effects of severe hyperkalemia and/or hypokalemia, such as peaked T wave, QRS widening, PR shortening (e.g., the PR interval is the time from the beginning of the P wave (atrial depolarization) to the beginning of the QRS complex (ventricular depolarization), and a shortened PR interval may indicate a certain disease), bradycardia (e.g., slower-than-expected heart rate), as well as other indices of cardiac function. Thus, because hyperkalemia and/or hypokalemia may be attributable to chronic kidney dysfunction, changes in ECG measurements reflecting hyperkalemia and/or hypokalemia may indicate a need for further testing for kidney dysfunction.

However, there exists a treatment paradox in that ECG devices, or the ability to interpret such ECG devices, are not readily accessible to most patients, for example in their own homes. Further, evidence is conflicting as to whether ECG findings are reliable, especially in patients with chronic hyperkalemia and/or hypokalemia. Additionally, changes in potassium levels happen well before changes in corresponding cardiac function that would be detected by ECG. Potassium levels change before cardiac function because changes in potassium levels are the underlying biological mechanism responsible for corresponding changes in cardiac function that may be detected by an ECG. As such, it is preferential to know if a potassium level would be changing to an unsafe range because this may indicate impending cardiac rhythm irregularities prior to these dangerous rhythm irregularities occurring in a patient. For example, high false-positives and high-false negatives are often seen with the use of ECG monitors. As used herein, a false positive is a result that indicates a given condition exists when the condition does not, and a false negative is a result that indicates a given condition does not exist when, in fact, the condition does exist. Further, such monitoring lacks the ability for continuous monitoring which is needed to provide a complete picture as to a patient's health.

Additionally, in some cases, T wave abnormalities detected by ECG monitoring may be attributed to factors unrelated to potassium levels. For example, T wave abnormalities occur as a result of subarachnoid hemorrhage, ischemic stroke, subdural hematoma, heart failure, myocardial edema, viral infection (e.g., Covid-19), traumatic brain injury, rare diseases, and specific oncologic pathways such as, but not limited to, pheochromocytoma. The value of using ECG alone to diagnose clinically significant hyperkalemia is further complicated by situations where T wave abnormalities are co-morbidities to kidney disease. In these cases, it is even more difficult to determine if the result of the T wave abnormality is a result of the kidney disease, or another clinical situation. Accordingly, T wave abnormalities recognized by ECG monitoring may not always be caused by decreased kidney function (e.g., abnormal potassium levels may, in some cases, be attributed to decreased kidney function). Thus, ECG alone may not provide sufficient information for assessing kidney health, nor provide sufficient information about the extent of kidney disease in a patient's body.

Again, adverse events associated with chronic kidney dysfunction are often related to high or low serum potassium levels (i.e., hyperkalemia or hypokalemia, respectively), which if left untreated, can create medical situations requiring urgent medical attention. Potassium is a crucial electrolyte and helps regulate fluid balance, muscle contractions, and nerve signaling in the human body. The kidney is primarily responsible for maintaining homeostasis of potassium levels in the body via control of potassium secretion, reabsorption, and excretion mechanisms. Thus, when kidney function declines, so may the control mechanisms for maintaining potassium homeostasis. Therefore, because potassium levels may indicate a change in kidney function, measuring the potassium levels of a patient is important for screening, diagnosing, staging, and monitoring CKD.

However, the current clinical standard for potassium measurement, and thus, for kidney health assessment, is a blood test. In some cases, whole blood samples are obtained by pricking the finger with a lancet. In some cases, blood samples are obtained using a venous blood draw. In venous blood sampling, a needle is inserted into a vein to collect a sample of blood for testing. Venous blood samples are often ordered for a patient on a weekly basis. Measuring whole blood, however, comes with a risk of hemolysis (e.g., the rupturing of red blood cells from external forces), particularly common in the finger prick method of blood collection, which can result in false positive measurements due to the high intracellular concentration of potassium that is released upon cell rupture. Of all routine blood tests, plasma/serum potassium measurement is one of the most sensitive measurements to the effect of hemolysis because red-cell potassium concentration is much higher than that of plasma (approximately 20 times higher). Accordingly, even slight hemolysis may cause a spuriously high plasma potassium concentration, which may make screening, diagnosing, and staging CKD based on potassium measurements unreliable.

Further, ensuring a patient continues to partake in such potassium monitoring activities, such as daily or weekly blood tests, may present a problem in itself. For patients with CKD, venous blood samples are often ordered on a weekly basis for potassium monitoring. However, such regular blood sampling may be costly, time-intensive, and painful for patients. As a result, patients may decide to forgo such potassium monitoring activities. And, a patient who forgoes engaging in such potassium monitoring activities, which help to stabilize the user's chronic condition, may fail to manage the condition outside of such tests. Where the condition is left unmanaged for too long, the patient' condition may significantly deteriorate, additional health issues may arise, and, in some cases, lead to an increased risk or likelihood of mortality.

Overall, existing diagnostic methods, such as those described above, suffer from a first technical problem of failing to continuously monitor the concentration of changing analytes, including potassium, to give a continuous readout. As used herein, the term “continuous” may mean fully continuous, semi-continuous, periodic, etc., and thus “continuously monitoring” may mean continuously monitoring, semi-continuously monitoring, periodically monitoring, etc. Such continuous monitoring of analytes is advantageous in screening, diagnosing, and staging a disease of a patient given the continuous measurements provide continuously up-to-date measurements, as well as information on trends and rates of analyte concentration changes over a continuous period. Such information may be used to make more informed decisions in the assessment of kidney health and treatment of kidney disease, and more particularly, chronic kidney disease (CKD).

Second, existing diagnostic methods suffer from another technical problem of failing to continuously monitor the concentration of a plurality of changing analytes, such as potassium and, e.g., glucose, simultaneously. In particular, the continuous monitoring of multiple analytes such as potassium, glucose, creatinine, lactate, urea (via blood urea nitrogen (BUN)), cystatin C, and/or C-peptide may provide additional insight when assessing the presence and/or severity of kidney disease, hyperkalemia, hypokalemia, and hyperglycemia and/or hypoglycemia in a patient. Further, the additional insight gained from using a combination of analytes, rather than just a single analyte, may help to increase the accuracy of the prediction, as well as make more informed patient-specific decisions and/or recommendations for the screening, diagnosing, staging, and monitoring of CKD.

As a result of these technical problems, diagnosing kidney disease, or a risk thereof, using conventional techniques may not only be inaccurate but also impossible, which, in some cases, might prove to be life threatening for a patient with such disease. Specifically, predicting the progression of chronic kidney disease (CKD) in a personalized manner with reasonable accuracy may be necessary given the dynamic and covert nature of kidney disease in its early stages, as well as patient heterogeneity. Thus, improved methods for screening, diagnosing, and staging CKD in a patient, as well as methods for understanding the interplay between CKD progression and measured analyte levels in the patient, are desired.

Accordingly, certain embodiments described herein provide a technical solution to the technical problems described above by providing decision support around kidney disease, and particularly, chronic kidney disease (CKD), using a continuous analyte monitoring system. Further, certain other embodiments, described herein provide a technical solution to the technical problems described above by providing decision support around kidney disease using a continuous analyte monitoring system, including, at least, a continuous potassium monitor (CPM). The decision support may be provided in the form of risk assessment (e.g., screening), diagnosis, staging, and/or monitoring kidney disease. As used herein, risk assessment may refer to an evaluation or estimation of present or future incidence of kidney dysfunction, kidney disease, one or more symptoms associated with kidney disease such as hyperkalemia and/or hypokalemia, and the like.

According to embodiments of the present disclosure, the decision support system presented herein is designed to provide a risk assessment, a diagnosis, and/or a staging for patients with, or at risk of, kidney disease as well as disease decision support to assist the patient in preventing, attenuating, and/or managing their kidney disease, or risk thereof. Providing kidney disease decision support may involve using large amounts of collected data, including for example, analyte data, patient information, and secondary sensor data mentioned above, to: (1) automatically detect and classify abnormal kidney function; (2) assess the risk of kidney disease; and (3) assess the presence and stage of kidney disease. In other words, the decision support system presented herein may offer information to direct and help improve care for patients with, or at risk, of kidney disease, such as CKD.

In certain embodiments, the decision support is provided in the form of a risk assessment of a patient developing kidney disease, e.g., CKD. In other words, decision support is provided in the form of a kidney disease screening. For example, periodic or continuous analyte measurements, as provided by one or more analyte sensors, may indicate an increased risk of developing kidney disease, such that additional diagnostic testing for kidney disease may be recommended. In another example, periodic or continuous analyte measurements, as provided by one or more analyte sensors, may indicate a low risk of developing kidney disease, such that additional diagnostic testing for kidney disease is not advised. Such analyte sensors, which may include a continuous glucose monitor (CGM) and/or a continuous potassium monitor (CPM), may be specifically used by a patient to screen for kidney disease according to methods described herein. Alternatively, where a patient is already utilizing an analyte sensor such as a CGM or CPM, a decision support system may periodically and/or continuously monitor or screen for increased kidney disease risk and alert a user (e.g., the patient) if risk of developing kidney disease is increased. For example, a CGM of a diabetes patient may be utilized to monitor for increased kidney disease risk in the diabetes patient. In some examples, upon such risk reaching or exceeding a predetermined threshold, a CPM may then be recommended for the patient to screen for kidney disease.

In certain embodiments, the decision support is provided in the form of a diagnosis of kidney disease, e.g., CKD. In other words, the decision support is provided in the form of an assessment of kidney disease presence and/or staging. For example, periodic or continuous analyte measurements, as provided by one or more analyte sensors, may indicate the presence and/or stage of kidney disease in a patient, which may be confirmed by additional diagnostic testing. In another example, periodic or continuous analyte measurements, as provided by one or more analyte sensors, may indicate healthy kidney function in a patient. For example, periodic or continuous analyte measurements may include the time a patient's analyte concentration revolves around a given set point over a 24-hour period, or a portion of the day (e.g., day time or night time). Additional examples may include time above a given threshold during a 24-hour day or during a portion of the day (e.g., day time or night time). Such indications may be based on analyte data, such as potassium levels, potassium level thresholds, potassium level rates of change, changes and rates of changes in potassium rates of change, average potassium levels, standard deviation of potassium levels, potassium clearance rates, personalized potassium data, and/or other changes in potassium data.

In certain examples, kidney disease decision support may further include assessing the risk of adverse health events associated with kidney disease, and/or providing patient-specific treatment recommendations for kidney disease. For example, in certain embodiments, the decision support is provided in the form of a risk assessment of mortality due to kidney disease. In certain embodiments, the decision support is provided in the form of a risk assessment of adverse health events such as hyperkalemia, hypokalemia, cardiac events, and the like. In certain embodiments, the decision support is provided in the form of a risk assessment of comorbidities, such as hypoglycemia, hyperglycemia, liver disease, and the like.

In certain embodiments, the decision support system may provide decision support to a patient based on a variety of collected data, including analyte data, patient information, secondary sensor data (e.g., non-analyte data), etc. For example, the analyte data may include continuously monitored potassium data in addition to other continuously monitored analyte data collected by a continuous analyte monitoring system, such as glucose, creatinine, urea (blood urea nitrogen (BUN)), inulin, dextran, saccharin, iothalamate, iohexol, 125I-iothanalamate, cystatin C, C-peptide, 51Cr-EDTA, lactate, asparagusic acid, polyfructosan, and betanin.

Potassium helps to regulate fluid balance, muscle contractions, and nerve signals in the body. A high-potassium diet may also help to reduce blood pressure and water retention, protect against stroke, and prevent osteoporosis and kidney stones. As mentioned herein, the kidneys play a major role in potassium homeostasis by renal mechanisms that transport and regulate potassium secretion, reabsorption and excretion. Thus, the continuously monitored analyte data may include potassium data as measured by a continuous potassium monitor (CPM) to indicate, or be used for determining, the patient's potassium levels and/or rates of change of the patient's potassium levels over time, for assessing kidney health and function for a patient.

According to certain embodiments, the decision support system described herein is designed to provide decision support in the form of risk assessment and treatment for CKD and/or potassium homeostasis. For example, in certain embodiments, the decision support system is designed to continuously measure serum potassium levels of a patient and provide recommendations for treatment, and specifically, potassium imbalance associated with kidney disease.

In certain embodiments, decision support risk assessment and treatment recommendations may be based on a patient's potassium levels, rates of change, trends, and/or thresholds. Different thresholds may be set based on risk(s) of, e.g., hyperkalemia and/or hypokalemia, available treatments, effectiveness of treatments, characteristics of the patient, patient activity, and/or the like.

Certain embodiments of the present disclosure also provide techniques and systems for correcting for a patient's potassium levels by using measurements associated with other analyte sensor data, the secondary sensor data, and/or other patient information, as further described below. As described above, the collected data also includes patient information, which may include information related to age, gender, family history of kidney disease, other health conditions, etc. Secondary sensor data may include accelerometer data, heart rate data (ECG, HRV, HR, etc.), temperature, blood pressure, time of sensor initiation or remaining sensor life relative to the initiation time, or any other sensor data other than analyte data.

In certain embodiments, the decision support system described herein may use various algorithms or artificial intelligence (AI) models, such as machine-learning models, trained based on patient-specific data, historical data, and/or population data to provide real-time decision support to a patient based on the collected information about the patient. For example, certain aspects are directed to algorithms and/or machine-learning models designed to assess the presence and severity of kidney disease in a patient. The algorithms and/or machine-learning models may be used in combination with one or more continuous analyte sensors, such as a CPM, to provide real-time kidney disease assessment and staging. In particular, the algorithms and/or machine-learning models may take into account parameters, such as potassium levels, rates of change of potassium levels of the patient over time, and other physiological parameters of a patient commonly associated with kidney disease, when screening, diagnosing, and staging CKD.

Based on these parameters, the algorithms and/or machine-learning models may provide a risk assessment of different CKD stages (and their corresponding severity), as well the progression a patient has made towards one or more of those CKD stages. The algorithms and/or machine-learning models may take into consideration population data, personalized patient-specific data, or a combination of both when screening, diagnosing, and staging CKD for a patient.

According to certain embodiments, prior to deployment, the machine learning models are trained with training data, e.g., including user-specific data and/or population data. As described in more detail herein, the population data may be provided in a form of a dataset including data records of historical patients with varying stages of kidney disease. Each data record may be used during training as input into the machine learning models to optimize such models to generate, as output, accurate predictions associated with CKD (e.g., predictions of kidney disease risk, presence, and/or severity in a patient, etc.).

The combination of a continuous analyte monitoring system with machine learning models and/or algorithms for screening, diagnosing, staging, and assessing risks of CKD provided by the decision support system described herein enables real-time diagnosis to allow early intervention. In particular, the decision support system may be used to provide an early alert of declining kidney function and/or deliver information about other complications related to the kidney. Early detection of such decompensation and/or other complications may allow for intervention at the earliest possible stage to ultimately improve kidney disease outcomes. For example, early intervention may reduce hospitalization, complications, and death, in some cases. In addition, potassium levels and changes in potassium levels provided by the continuous analyte monitoring system may be used as input into the machine learning models and/or algorithms to triage patients for more urgent care. In patients at risk for cardiac arrhythmia episodes and/or death, sudden increases in potassium may be used to inform urgent medical intervention, even before patients are able to report noticeable physiologic symptoms.

In addition, through the combination of a continuous analyte monitoring system with machine learnings and/or algorithms for screening, diagnosing, staging, and assessing risk of CKD, the decision support system described herein may provide the necessary accuracy and reliability patients expect. For example, biases, human errors, and emotional influence may be minimized when assessing the presence and severity of kidney disease in patients. Further, machine learning models and algorithms in combination with analyte monitoring systems may provide insight into patterns and/or trends of decreasing health of a patient, at least with respect to the kidney and/or heart, which may have been previously missed. Accordingly, the decision support system described herein may assist in the identification of kidney health for CKD screening, diagnosis, preventive, and treatment purposes.

### Example Decision Support System Including an Example Analyte Sensor

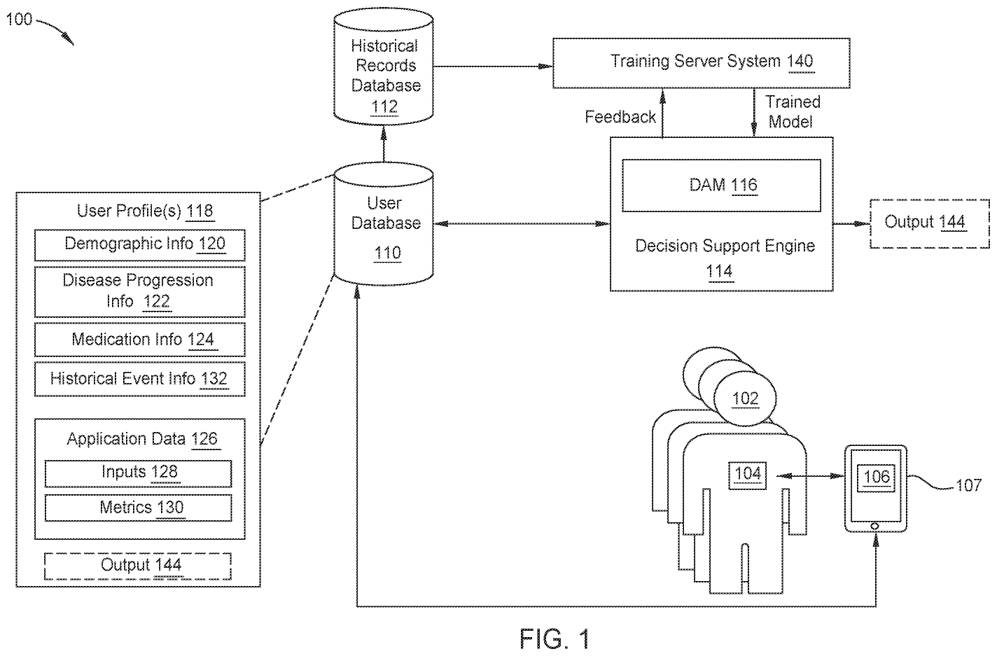

FIG. 1 illustrates an example decision support system 100 for screening, diagnosing, staging, and treating kidney disease, and particularly, chronic kidney disease (CKD), in relation to users 102 (individually referred to herein as a user and collectively referred to herein as users), using a continuous analyte monitoring system 104, including one or more analyte sensors. A user, in certain embodiments, may be the patient or, in some cases, the patient's caregiver. In certain embodiments, decision support system 100 includes continuous analyte monitoring system 104, a display device 107 that executes application 106, a decision support engine 114, a user database 110, a historical records database 112, a training server system 140, and a decision support engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. Analytes for measurement by the devices and methods may include, but may not be limited to, potassium, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-3 hydroxy-cholic acid; cortisol; creatine kinase; creatine kinase MM isoenzyme; cyclosporin A; cystatin C; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sisomicin; somatomedin C; specific antibodies recognizing any one or more of the following that may include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain implementations. Ions are a charged atoms or compounds that may include the following (sodium, potassium, calcium, chloride, nitrogen, or bicarbonate, for example). The analyte can be naturally present in the biological fluid, for example, a metabolic product, a hormone, an antigen, an antibody, an ion and the like. Alternatively, the analyte can be introduced into the body or exogenous, for example, a contrast agent for imaging, a radioisotope, a chemical agent, a fluorocarbon-based synthetic blood, a challenge agent analyte (e.g., introduced for the purpose of measuring the increase and or decrease in rate of change in concentration of the challenge agent analyte or other analytes in response to the introduced challenge agent analyte), or a drug or pharmaceutical composition, including but not limited to exogenous insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include potassium, and in some cases glucose, creatinine, urea (blood urea nitrogen (BUN)), inulin, dextran, saccharin, iothalamate, iohexol, 125I-iothanalamate, cystatin C, C-peptide, 51Cr-EDTA, lactate, asparagusic acid, polyfructosan, and betanin, other analytes listed, but not limited to, above may also be considered and measured by, for example, analyte monitoring system 104.

In certain embodiments, continuous analyte monitoring system 104 is configured to continuously measure one or more analytes and transmit the analyte measurements to an electric medical records (EMR) system (not shown in FIG. 1). An EMR system is a software platform which allows for the electronic entry, storage, and maintenance of digital medical data. An EMR system is generally used throughout hospitals and/or other caregiver facilities to document clinical information on patients over long periods. EMR systems organize and present data in ways that assist clinicians with, for example, interpreting health conditions and providing ongoing care, scheduling, billing, and follow up. Data contained in an EMR system may also be used to create reports for clinical care and/or disease management for a patient. In certain embodiments, the EMR may be in communication with decision support engine 114 (e.g., via a network) for performing the techniques described herein. In particular, as described herein, decision support engine 114 may obtain data associated with a user, use the obtained data as input into one or more trained model(s), and output a prediction. In some cases, the EMR may provide the data to decision support engine 114 to be used as input into one or more models, e.g., ML models. Further, in some cases, decision support engine 114, after making a prediction, may provide the output prediction to the EMR.

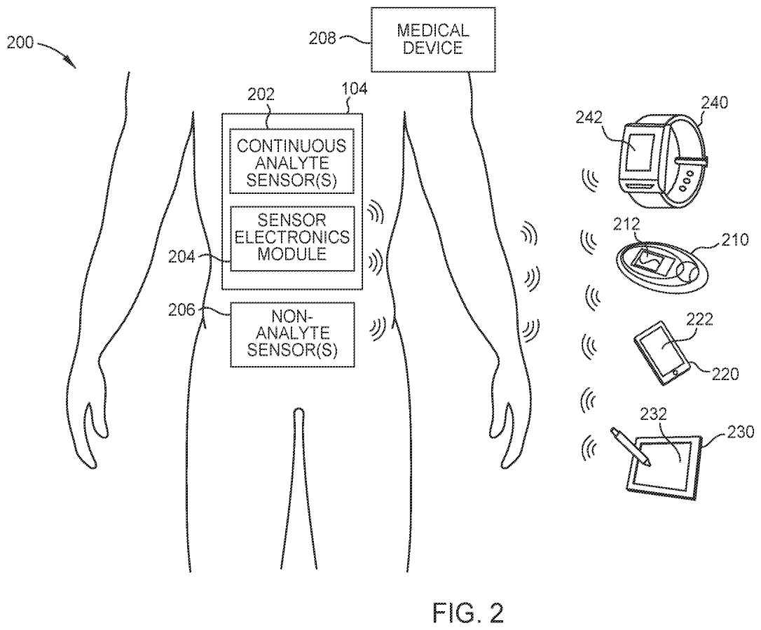

In certain embodiments, continuous analyte monitoring system 104 is configured to continuously measure one or more analytes and transmit the analyte measurements to display device 107 for use by application 106. In some embodiments, continuous analyte monitoring system 104 transmits the analyte measurements to display device 107 through a wireless connection (e.g., Bluetooth connection). In certain embodiments, display device 107 is a smart phone. However, in certain other embodiments, display device 107 may instead be any other type of computing device such as a laptop computer, a smart watch, a tablet, or any other computing device capable of executing application 106. In some embodiments, continuous analyte monitoring system 104 and/or analyte sensor application 106 transmit the analyte measurements to one or more other individuals having an interest in the health of the patient (e.g., a family member or physician for real-time treatment and care of the patient). Continuous analyte monitoring system 104 may be described in more detail with respect to FIG. 2.

Application 106 is a mobile health application that is configured to receive and analyze analyte measurements from analyte monitoring system 104. In particular, application 106 stores information about a user, including the user's analyte measurements, in a user profile 118 associated with the user for processing and analysis, as well as for use by decision support engine 114 to provide decision support recommendations or guidance to the user.

Decision support engine 114 refers to a set of software instructions with one or more software modules, including data analysis module (DAM) 116. In certain embodiments, decision support engine 114 executes entirely on one or more computing devices in a private or a public cloud. In such embodiments, application 106 communicates with decision support engine 114 over a network (e.g., Internet). In some other embodiments, decision support engine 114 executes partially on one or more local devices, such as display device 107, and partially on one or more computing devices in a private or a public cloud. In some other embodiments, decision support engine 114 executes entirely on one or more local devices, such as display device 107. As discussed in more detail herein, decision support engine 114 may provide decision support recommendations to the user via application 106. Decision support engine 114 provides decision support recommendations based on information included in user profile 118.

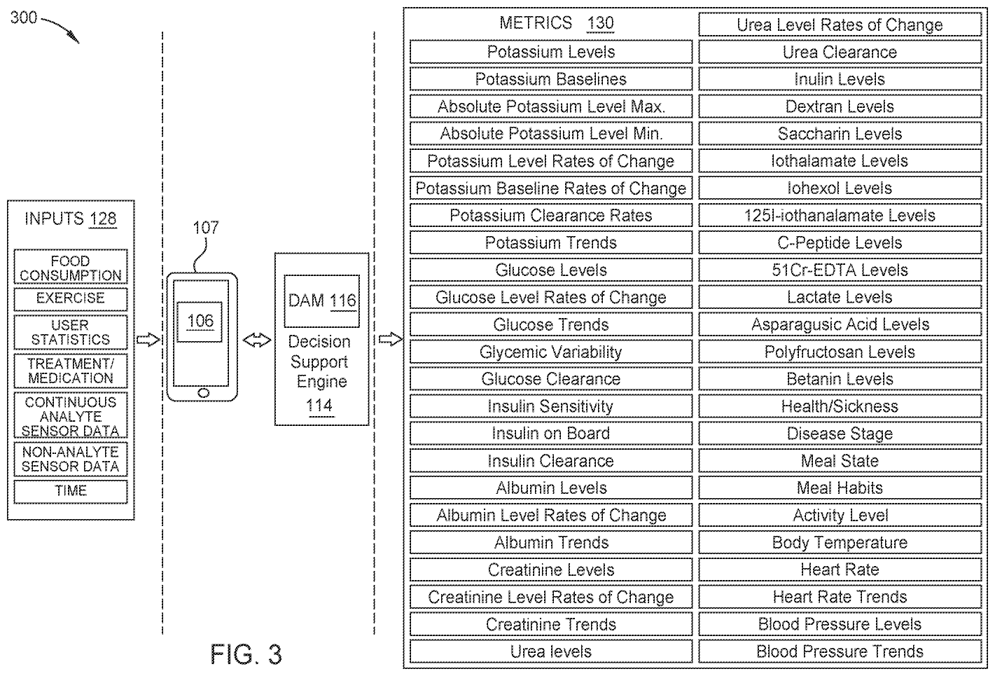

User profile 118 may include information collected about the user from application 106. For example, application 106 provides a set of inputs 128, including the analyte measurements received from continuous analyte monitoring system 104, that are stored in user profile 118. In certain embodiments, inputs 128 provided by application 106 include other data in addition to analyte measurements received from continuous analyte monitoring system 104. For example, application 106 may obtain additional inputs 128 through manual user input, one or more other non-analyte sensors or devices, other applications executing on display device 107, etc. Non-analyte sensors and devices include one or more of, but are not limited to, an insulin pump, an electrocardiogram (ECG) sensor or heart rate monitor, a blood pressure sensor, a respiratory sensor, a thermometer, a peritoneal dialysis machine, a hemodialysis machine, sensors or devices provided by display device 107 (e.g., accelerometer, camera, global positioning system (GPS), heart rate monitor, etc.) or other user accessories (e.g., a smart watch), or any other sensors or devices that provide relevant information about the user. Inputs 128 of user profile 118 provided by application 106 are described in further detail below with respect to FIG. 3.

DAM 116 of decision support engine 114 is configured to process the set of inputs 128 to determine one or more metrics 130. Metrics 130, discussed in more detail below with respect to FIG. 3, may, at least in some cases, be generally indicative of the health or state of a user, such as one or more of the user's physiological state, trends associated with the health or state of a user, etc. In certain embodiments, metrics 130 may then be used by decision support engine 114 as input for providing guidance to a user. As shown, metrics 130 are also stored in user profile 118. In certain embodiments, a user's glucose metrics may include glucose levels, time-stamped glucose levels, glucose rate(s) of change, glucose trend(s), a mean glucose level, a glucose management indicator (GMI), a glycemic variability, a time in range (TIR), a glucose clearance rate, minimum and maximum glucose levels, a glucose autocorrelation score, a glucose set point, etc.

User profile 118 also includes demographic info 120, disease progression info 122, and/or medication info 124. In certain embodiments, such information may be provided through user input or obtained from certain data stores (e.g., electronic medical records (EMRs), etc.). In certain embodiments, demographic info 120 may include one or more of the user's age, body mass index (BMI), ethnicity, gender, etc. In certain embodiments, disease progression info 122 may include information about a disease of a user, such as whether the user has been previously diagnosed with acute kidney injury (AKI), a condition that places the user at risk of developing AKI (e.g., myocardial infarction, rhabdomyolysis, sepsis or other infectious disease, hypo perfusion such as from blood loss, or other diseases of the kidney), or chronic kidney disease (CKD), or have had a history of hyperkalemia, hypokalemia, hyperglycemia, hypoglycemia, etc. In certain embodiments, information about a user's disease may also include the length of time since diagnosis, the level of disease control, level of compliance with disease management therapy, predicted kidney function, other types of diagnosis (e.g., heart disease, obesity) or measures of health (e.g., heart rate, exercise, stress, sleep, etc.), and/or the like.

In certain embodiments, medication info 124 may include information about the amount, frequency, and type of a medication taken by a user. In certain embodiments, the amount, frequency, and type of a medication taken by a user is time-stamped and correlated with the user's analyte levels, thereby, indicating the impact the amount, frequency, and type of the medication had on the user's analyte levels. In certain embodiments, medication information may include information about the consumption of one or more diuretics. Diuretics may be prescribed to a patient for the purpose of treating excessive fluid accumulation caused by, for example, congestive heart failure (CHF), liver failure, and/or nephritic syndrome. For example, CHF is a condition in which the heart is unable to efficiently pump blood to meet the body's oxygen and nutrient needs. The inability of the heart to efficiently pump blood impairs normal blood circulation and leads to excess fluid in the blood. The excess fluid leaks out of the blood vessels and accumulates in the lungs and other tissues. Accordingly, a patient may be prescribed diuretics to help the kidneys flush out the excess fluid and maintain normal blood volume.

Different types of diuretics prescribed to a patient may include loop diuretics, thiazide and thiazide-like diuretics, and potassium-sparing diuretics. Loop diuretics inhibit a protein found in a part of the nephron known as the loop of Henle. Loop diuretics may include, for example, Furosemide (Lasix), Bumetanide (Bumex), Torsemide (Demadex), Ethacrynic acid (Edecrin). Thiazide diuretics are commonly used to treat high blood pressure (hypertension), but also to manage heart failure. Thiazide diuretics inhibit a different protein than the loop diuretics do, which also helps in mineral reabsorption. Thiazide diuretics may include, for example, Chlorothiazide (Diuril), Hydrochlorothizaide (Hydrodiuril), Metolazone (Zytonix). Lastly, potassium sparing diuretics (e.g., such as Spironolactone) are weak diuretics used to increase the amount of fluid passed from the body in urine, while also preventing too much potassium from being lost from the body in the urine. As described in more detail below, decision support system 100 may be configured to use medication info 124 to determine optimal diuretics to be prescribed to different users. In particular, decision support system 100 may be configured to identify one or more optimal diuretics for prescription based on the health of the patient when one or more diuretics are prescribed, as well as the condition(s) of the patient to be treated.

In certain embodiments, medication information may include information about consumption of one or more drugs known to damage the kidney. One or more drugs known to damage the kidney may include nonsteroidal anti-inflammatory drugs (NSAIDS) such as ibuprofen (e.g., Advil, Motrin) and naproxen (e.g., Aleve), vancomycin, iodinated radiocontrast (e.g., refers to any contrast dyes used in diagnostic testing), angiotensin-converting enzyme (ACE) such as lisinopril, enalapril, and ramipril, aminoglycoside antibiotics such as neomycdin, gentamicin, tobramycin, and amikacin, antiviral human immunodeficiency virus (HIV) medications, zoledronic acid (e.g., Zometa, Reclast), foscarnet, lithium, and the like.

In certain embodiments, medication information may include information about consumption of one or more drugs known to control the complications of kidney disease. One or more drugs known to control the complications of kidney disease may include medications to lower blood pressure and preserve kidney function such as ACE inhibitors or angiotensin II receptor blockers, medications to treat anemia such as supplements of the hormone erythropoietin, medications used to lower cholesterol levels such as statins, medications used to prevent weak bones such as calcium and vitamin D supplements, phosphate binders, and the like.

In certain embodiments, user profile 118 is dynamic because at least part of the information that is stored in user profile 118 may be revised over time and/or new information may be added to user profile 118 by decision support engine 114 and/or application 106. Accordingly, information in user profile 118 stored in user database 110 provides an up-to-date repository of information related to a user.

User database 110, in some embodiments, refers to a storage server that operates in a public or private cloud. User database 110 may be implemented as any type of datastore, such as relational databases, non-relational databases, key-value datastores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 includes user profiles 118 associated with a plurality of users who similarly interact with application 106 executing on the display devices 107 of the other users. User profiles stored in user database 110 are accessible to not only application 106, but decision support engine 114, as well. User profiles in user database 110 may be accessible to application 106 and decision support engine 114 over one or more networks (not shown). As described above, decision support engine 114, and more specifically DAM 116 of decision support engine 114, can fetch inputs 128 from user database 110 and compute a plurality of metrics 130 which can then be stored as application data 126 in user profile 118.

In certain embodiments, user profiles 118 stored in user database 110 may also be stored in historical records database 112. User profiles 118 stored in historical records database 112 may provide a repository of up-to-date information and historical information for each user of application 106. Thus, historical records database 112 essentially provides all data related to each user of application 106, where data is stored according to an associated timestamp. The timestamp associated with information stored in historical records database 112 may identify, for example, when information related to a user has been obtained and/or updated.

Further, historical records database 112 may maintain time series data collected for users over a period of time, including for users who use continuous analyte monitoring system 104 and application 106. For example, analyte data for a user who has used continuous analyte monitoring system 104 and application 106 for a period of five years to manage the user's kidney condition may have time series analyte data associated with the user maintained over the five-year period.

Further, in certain embodiments, historical records database 112 may include data for one or more patients who are not users of continuous analyte monitoring system 104 and/or application 106. For example, historical records database 112 may include information (e.g., user profile(s)) related to one or more patients analyzed by, for example, a healthcare physician (or other known method), and not previously diagnosed with kidney disease (e.g., CKD), as well as information (e.g., user profile(s)) related to one or more patients who were analyzed by, for example, a healthcare physician (or other known method) and were previously diagnosed with (varying types and stages of) kidney disease. Data stored in historical records database 112 may be referred to herein as population data.

Data related to each patient stored in historical records database 112 may provide time series data collected over the disease lifetime of the patient. For example, the data may include information about the patient prior to being diagnosed with kidney disease and information associated with the patient during the lifetime of the disease, including information related to each stage of kidney disease (e.g., CKD) as it progressed and/or regressed in the patient, as well as information related to other diseases or conditions, such as hyperkalemia, hypokalemia, diabetes, heart conditions and diseases, or similar diseases that are co-morbid in relation to kidney disease. Such information may indicate symptoms of the patient, physiological states of the patient, potassium levels of the patient, glucose levels of the patient, creatinine levels of patient, BUN levels of the patient, cystatin C levels of the patient, C-peptide levels of the patient, albumin levels of the patient, creatinine levels of the patient, inulin levels of the patient, dextran levels of the patient, saccharin levels of the patient, iothalamate levels of the patient, iohexol levels of the patient, 125I-iothanalamate levels of the patient, 51Cr-EDTA levels of the patient, lactate levels of the patient, asparagusic acid levels of the patient, polyfructosan levels of the patient, betanin levels of the patient, states/conditions of one or more organs of the patient, habits of the patient (e.g., activity levels, food consumption, etc.), medication prescribed, etc., throughout the lifetime of the disease.

Although depicted as separate databases for conceptual clarity, in some embodiments, user database 110 and historical records database 112 may operate as a single database. That is, historical and current data related to users of continuous analyte monitoring system 104 and application 106, as well as historical data related to patients that were not previously users of continuous analyte monitoring system 104 and application 106, may be stored in a single database. The single database may be a storage server that operates in a public or private cloud.

As mentioned previously, decision support system 100 is configured to screen, diagnose, and stage kidney disease for a user using continuous analyte monitoring system 104 including one or more analyte sensors. In certain embodiments, continuous analyte monitoring system 104 includes, at least a continuous potassium monitor (CPM). In certain embodiments, decision support engine 114 is configured to provide real-time and or non-real-time decision support around kidney disease to the user and or others, including but not limited, to healthcare providers, family members of the user, caregivers of the user, researchers, artificial intelligence (AI) engines, and/or other individuals, systems, and/or groups supporting care or learning from the data. In particular, decision support engine 114 may be used to collect information associated with a user in user profile 118 stored in user database 110, to perform analytics thereon for predicting the presence and/or severity of kidney disease for the user and/or predicting the likelihood of the user developing kidney disease within a certain time period, as well as providing one or more recommendations for treatment based, at least in part, on the predictions. User profile 118 may be accessible to decision support engine 114 over one or more networks (not shown) for performing such analytics.

In certain embodiments, decision support system 100 is designed to predict the risk or likelihood of, or the presence and/or severity of, kidney disease in real-time (including near real-time), or within a specified period of time for a patient. In certain embodiments, to enable such prediction, decision support engine 114 is configured to collect information associated with a user in user profile 118 stored in user database 110, to perform analytics thereon for: (1) automatically detecting and classifying abnormal kidney function; (2) assessing the risk of kidney disease; and (3) assessing the presence and stage of kidney disease.

In certain embodiments, decision support engine 114 may utilize one or more trained machine learning models capable of determining the probability of the presence and/or severity of kidney disease for a user based on information provided by user profile 118. In the illustrated embodiment of FIG. 1, decision support engine 114 may utilize trained machine learning model(s) provided by a training server system 140. Although depicted as a separate server for conceptual clarity, in some embodiments, training server system 140 and decision support engine 114 may operate as a single server. That is, the model may be trained and used by a single server, or may be trained by one or more servers and deployed for use on one or more other servers. In certain embodiments, the model may be trained on one or many virtual machines (VMs) running, at least partially, on one or many physical servers in relational and or non-relational database formats.

Training server system 140 is configured to train the machine learning model(s) using training data, which may include data (e.g., from user profiles) associated with one or more patients (e.g., users or non-users of continuous analyte monitoring system 104 and/or application 106) previously diagnosed with varying stages of kidney disease, as well as patients not previously diagnosed with kidney disease (e.g., healthy patients). The training data may be stored in historical records database 112 and may be accessible to training server system 140 over one or more networks (not shown) for training the machine learning model(s). The training data may also, in some cases, include user-specific data for a user over time.

The training data refers to a dataset that has been featurized and labeled. For example, the dataset may include a plurality of data records, each including information corresponding to a different user profile stored in user database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an illustrative example, each relevant characteristic of a user, which is reflected in a corresponding data record, may be a feature used in training the machine learning model. Such features may include age, gender, change (e.g., delta) in analyte levels (e.g., potassium levels) from a first timestamp to a second timestamp, change (e.g., delta) in kidney disease stage or severity from a first timestamp to a second timestamp, change (e.g., delta) in analyte thresholds (e.g., potassium thresholds) of a user suffering from kidney disease from a first timestamp to a subsequent timestamp, the derivative of the measured linear system of analyte measurement (e.g., potassium measurement) at a point at a specific timestamp and or the difference in derivatives to determine rates of change in the slope of the increase or decrease in analyte values (e.g., potassium values), etc. In addition, the data record is labeled with an indication as to a kidney disease diagnosis, an assigned disease severity, and/or an identified risk of kidney disease, etc., associated with a patient of the user profile.

The model(s) are then trained by training server system 140 using the featurized and labeled training data. In particular, the features of each data record may be used as input into the machine learning model(s), and the generated output may be compared to label(s) associated with the corresponding data record. The model(s) may compute a loss based on the difference between the generated output and the provided label(s). This loss is then used to modify the internal parameters or weights of the model. By iteratively processing each data record corresponding to each historical patient, in certain embodiments, the model(s) may be iteratively refined to generate accurate predictions associated with kidney disease risk, presence, progression, improvement (e.g., regression), and severity in a patient. Further, in certain other embodiments, by iteratively processing each data record corresponding to each historical patient, in certain embodiments, the model(s) may be iteratively refined to generate accurate predictions the risk and/or presence of CKD.

As illustrated in FIG. 1, training server system 140 deploys these trained model(s) to decision support engine 114 for use during runtime. For example, decision support engine 114 may obtain user profile 118 associated with a user, use information in user profile 118 as input into the trained model(s), and output a prediction. The prediction may be indicative of the presence and/or severity of kidney disease for the user in real-time or within a certain time (e.g., shown as output 144 in FIG. 1). Output 144 generated by decision support engine 114 may also provide one or more recommendations for treatment based on the predictions. Output 144 may be provided to the user (e.g., through application 106), to a user's caretaker (e.g., a parent, a relative, a guardian, a teacher, a nurse, etc.), to a user's physician, or any other individual that has an interest in the wellbeing of the user for purposes of improving the user's health, such as, in some cases by effectuating the recommended treatment.

In certain embodiments, the user's own data is used to personalize the one or more models that are initially trained based on population data. For example, a model (e.g., trained using population data) may be deployed for use by decision support engine 114 to predict the presence and/or severity of kidney disease of a specific user. Some time after making a prediction using the model, decision support engine 114 may be configured to ask the user, or a caretaker, physician, etc., whether the predicted presence and/or severity of kidney disease was confirmed by, e.g., other diagnostic methods, and/or decision support engine 114 may use one or more diagnostic tests to confirm the diagnosis. In some cases, the user's answer and/or results from the diagnostic test(s) performed may deny the presence of kidney disease. Accordingly, the model may continue to be retrained and/or personalized using the user's answer, diagnostic test results, and/or physiological parameters of the user used as input into the model to personalize the model for the user.

In certain embodiments, output 144 generated by decision support engine 114 may be stored in user profile 118. In certain embodiments, output 144 may be patient-specific treatment decisions or recommendations for preventing one or more kidney disease predictions from occurring. For example, in certain embodiments, output 144 may be a prediction as to the presence and/or severity of chronic kidney disease (CKD) in a user. In certain embodiments, output 144 may be a prediction as to the risk of a user having CKD. In certain embodiments, output 144 may be a prediction as to the risk of a user having hyperkalemia and/or hypokalemia. In certain embodiments, output 144 may be a prediction as to a mortality risk of the patient. In certain embodiments, output 144 may be patient-specific treatment decisions or recommendations for CKD for the patient. In still further embodiments, output 144 may be a prediction as to the presence and/or severity of acute kidney injury (AKI), a prediction as to the risk of a user having AKI, patient-specific treatment decisions or recommendations for AKI for the patient, etc. Output 144 stored in user profile 118 may be continuously updated by decision support engine 114. Accordingly, previous diagnoses and/or physiological parameters of the user associated with kidney disease, originally stored as outputs 144 in user profile 118 in user database 110 and then passed to historical records database 112, may provide an indication of the progression of CKD/AKI (or other types of kidney disease) in a user over time, as well as provide an indication as to the effectiveness of different treatments (e.g., medications) recommended to a user to help stop progression of the disease.

In certain embodiments, a user's own historical data may be used to provide decision support and insight around the user's kidney function and/or disease. For example, a user's historical data may be used by an algorithm as a baseline to indicate improvements or deterioration in the user's kidney function. As an illustrative example, a user's data from two weeks prior may be used as a baseline that can be compared with the user's current data to identify whether the user's kidney function has improved or deteriorated. In certain embodiments, the user's own historical data may be used by training server system 140 to train a personalized model that may further be able to predict or project out the user's kidney function or the kidney's future improvement/deterioration based on the user's recent pattern of data (e.g., exercise data, food consumption data, etc.).

In certain embodiments, the model may be trained to provide lifestyle recommendations, exercise recommendations, food intake recommendations, medication recommendations, and other types of decision support recommendations to help the user prevent onset and/or progression of kidney disease, treat symptoms, and improve their kidney health and function based on the user's historical data, including how different types of medication, food, and treatment (e.g., such as dialysis) have impacted the user's kidney function in the past. In certain embodiments, the model may be trained to predict the underlying cause of certain improvements or deteriorations in the patient's kidney function. For example, application 106 may display a user interface with a graph that shows the patient's kidney functionality or a score thereof with trend lines and indicate, e.g., retrospectively, what caused the functionality of the kidney to suffer at certain points in time (e.g., excess potassium intake, ingestion of non-potassium sparing diuretics, etc.).

FIG. 2 is a diagram 200 conceptually illustrating an example continuous analyte monitoring system 104 including example continuous analyte sensor(s) with sensor electronics, in accordance with certain aspects of the present disclosure. For example, system 104 may be configured to continuously monitor one or more analytes of a user, in accordance with certain aspects of the present disclosure.

Generally, real-time or continuous measurements of analyte levels, rates of change, trends, clearance rates, and/or other analyte data, as measured in interstitial fluid or blood by a continuous analyte monitoring system, can be used to indicate a change in kidney function (e.g., either impairment or improvement of function). Such data can indicate a change in kidney function well in advance of conventional kidney disease diagnostic tools. Therefore, continuous analyte monitoring may provide earlier, and/or improved screening, diagnosis, prognosis, and/or staging of kidney disease as compared to conventional diagnostics.

In certain embodiments, clinical indicators may be used to determine whether a continuous analyte monitoring system, e.g., continuous analyte monitoring system 104, may be needed to assess a risk, presence, and/or stage of kidney disease in a patient. In one example, such clinical indicators include a GFR test, including mGFR and eGFR measurements. GFR testing may generally indicate the presence, risk, or likelihood of kidney dysfunction, and thus, after taking a GFR test, confirmation of the GFR results or screening for kidney dysfunction with a continuous analyte monitor may be desirable.

In yet another example, clinical indicators may include an annual screening. For example, at a patient's annual screening, with and without other risk factors for kidney disease, it may be desirable to initiate screening for kidney dysfunction with a continuous analyte monitor. In certain cases, a patient may be prescribed to use a continuous analyte monitor to screen for kidney disease for a period of time (e.g., two to four weeks or more).

In yet another example, clinical indicators may include previous symptoms of kidney dysfunction. For a patient experiencing, or with a history of experiencing, symptoms of kidney dysfunction, it may be desirable to initiate screening for kidney dysfunction with a continuous analyte monitor. In another example, clinical indicators may include previous symptoms of potassium imbalance. Generally, symptoms of potassium imbalance may include numbness/tingling, shortness of breath, chest pain, muscle weakness, and the like.

In yet another example, clinical indicators may include prescribed or taken medications. For a patient taking certain medications associated with kidney dysfunction, or medication associated with causing renal injury, it may be desirable to monitor and/or screen for kidney dysfunction. For example, a patient on a medication known to cause renal injury may use a continuous analyte monitor to screen for kidney dysfunction, which may have been caused by the medication.

In yet another example, clinical indicators may include comorbidities often associated with, and/or increasing the risk of, kidney dysfunction. Comorbidities associated with kidney disease include cardiovascular disease, obesity, liver disease, hypertension, and/or diabetes.

In yet another example, clinical indicators may include patient risk factors that may increase a patient's risk of kidney dysfunction and/or disease. Such risk factors for kidney disease may include age, history of low birth weight, and/or family history of kidney disease.

In yet another example, clinical indicators may include physiological parameters that may increase a patient's risk of kidney dysfunction. Such physiological parameters include abnormal potassium levels (e.g., from blood measurements), CPM data indicating further screening, diagnosis, and/or staging of kidney disease is desirable, and/or CGM data indicating screening, diagnosis, and/or staging of kidney disease is desirable.

In yet another example, clinical indicators may include adverse health events that may increase a patient's risk of kidney dysfunction. Such adverse health events may include hyperkalemia, hypokalemia, cardiac events, hyperglycemia, and/or hypoglycemia.

In certain embodiments, continuous analyte monitoring system 104 may be utilized as a short-term diagnostic tool (i.e., 10-14 days) to screen, diagnose, and/or stage a patient with kidney disease. For example, a triggering action (e.g., triggering while wearing an analyte sensor), may indicate utility for a patient to wear an analyte sensor (continuous or non-continuous) for a short time period to provide screening, diagnosis and/or staging of kidney disease. In one instance, a patient may wear a short-term continuous or non-continuous analyte sensor at each annual physical to screen for kidney disease. In another instance, a patient may wear a short-term continuous or non-continuous analyte sensor periodically (e.g., every 4 weeks), to monitor presence/stage of kidney disease. In yet another instance, a patient may wear a short-term continuous or non-continuous analyte sensor to determine a stage of kidney disease.

In yet another example, a patient may wear a short-term continuous or non-continuous analyte sensor to confirm and/or provide additional data for a clinical diagnosis of kidney disease, or to eliminate a possible clinical kidney disease diagnosis. For instance, where a patient's clinical creatinine level may indicate possible kidney dysfunction, the patient may wear a short-term continuous or non-continuous analyte sensor to monitor creatinine levels (or other analyte levels) and confirm kidney disease. In another instance, a patient may wear a short-term continuous or non-continuous analyte sensor to monitor risk factors associated with kidney disease, such as glucose imbalance, e.g., hypoglycemia, or potassium imbalance, e.g., hyperkalemia. In another instance, a patient may wear a short-term continuous or non-continuous analyte sensor during a mGFR test to provide additional insight into mGFR results.

In yet another example, a patient may wear a continuous analyte sensor, such as a CPM, whereby the monitor periodically functions as a short-term monitor to screen, diagnose, and/or stage a patient with kidney disease. For instance, a patient with diabetes using a continuous analyte monitor capable of sensing both glucose and potassium may utilize the continuous potassium monitoring functionality as a short-term monitoring tool for kidney disease.

In certain embodiments, continuous analyte monitoring system 104 may be utilized as a long-term diagnostic tool (i.e., greater than 14 days) to screen, diagnose, and/or stage a patient with kidney disease. For example, a patient at high risk for kidney disease and/or adverse events may utilize a continuous analyte sensor to continually screen, diagnose, and/or stage the patient for kidney disease for weeks, months, etc. For instance, a patient with stage 3 kidney disease may utilize a CPM to monitor their kidney disease and/or indicate worsening or improving kidney function over long periods of time.

Returning now to FIG. 2, continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 may be in wireless communication (e.g., directly or indirectly) with one or more of display devices 210, 220, 230, and 240. In certain embodiments, sensor electronics module 204 may also be in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

In certain embodiments, a continuous analyte sensor 202 may comprise a sensor for detecting and/or measuring analyte(s). The continuous analyte sensor 202 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 202 may be configured to continuously measure analyte levels of a user using one or more measurement techniques, such as enzymatic, chemical, physical, electrochemical, spectrophotometric, polarimetric, calorimetric, iontophoretic, radiometric, immunochemical, and the like. In certain aspects the continuous analyte sensor 202 provides a data stream indicative of the concentration of one or more analytes in the user. The data stream may include raw data signals, which are then converted into a calibrated and/or filtered data stream used to provide estimated analyte value(s) to the user.

In certain embodiments, continuous analyte sensor 202 may be a multi-analyte sensor, configured to continuously measure multiple analytes in a user's body. For example, in certain embodiments, the continuous multi-analyte sensor 202 may be a single sensor configured to measure potassium, glucose, lactate, ketones, creatinine, blood urea nitrogen (BUN), cystatin C, C-peptide, albumin, inulin, dextran, saccharin, iothalamate, iohexol, 125I-iothanalamate, 51Cr-EDTA, asparagusic acid, polyfructosan, and/or betanin in the user's body.

In certain embodiments, one or more multi-analyte sensors may be used in combination with one or more single analyte sensors. As an illustrative example, a multi-analyte sensor may be configured to continuously measure potassium and glucose and may, in some cases, be used in combination with an analyte sensor configured to measure only, for example, BUN levels or lactate levels. Information from each of the multi-analyte sensor(s) and single analyte sensor(s) may be combined to provide kidney disease decision support using methods described herein.

In certain embodiments, sensor electronics module 204 includes electronic circuitry associated with measuring and processing the continuous analyte sensor data, including prospective algorithms associated with processing and calibration of the sensor data. Sensor electronics module 204 can be physically connected to continuous analyte sensor(s) 202 and can be integral with (non-releasably attached to) or releasably attachable to continuous analyte sensor(s) 202. Sensor electronics module 204 may include hardware, firmware, and/or software that enables measurement of levels of analyte(s) via a continuous analyte sensor(s) 202. For example, sensor electronics module 204 can include a potentiostat, a power source for providing power to the sensor, other components useful for signal processing and data storage, and a telemetry module for transmitting data from the sensor electronics module to one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 210, 220, 230, and/or 240 are configured for displaying displayable sensor data, including analyte data, which may be transmitted by sensor electronics module 204. Each of display devices 210, 220, 230, or 240 can include a display such as a touchscreen display 212, 222, 232, /or 242 for displaying sensor data to a user and/or receiving inputs from the user. For example, a graphical user interface (GUI) may be presented to the user for such purposes. In some embodiments, the display devices may include other types of user interfaces such as a voice user interface instead of, or in addition to, a touchscreen display for communicating sensor data to the user of the display device and/or receiving user inputs. Display devices 210, 220, 230, and 240 may be examples of display device 107 illustrated in FIG. 1 used to display sensor data to a user of FIG. 1 and/or receive input from the user.

In some embodiments, one, some, or all of the display devices are configured to display or otherwise communicate the sensor data as it is communicated from the sensor electronics module (e.g., in a data package that is transmitted to respective display devices), without any additional prospective processing required for calibration and real-time display of the sensor data.

The plurality of display devices may include a custom display device specially designed for displaying certain types of displayable sensor data associated with analyte data received from sensor electronics module. In certain embodiments, the plurality of display devices may be configured for providing alerts/alarms based on the displayable sensor data. Display device 210 is an example of such a custom device. In some embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch, medical device 208 (e.g., an insulin delivery device or a blood glucose meter), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end user) for each particular display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data. In certain embodiments, the type of alarms customized for each particular display device, the number of alarms customized for each particular display device, the timing of alarms customized for each particular display device, and/or the threshold levels configured for each of the alarms (e.g., for triggering) are based on output 144 (e.g., as mentioned, output 144 may be indicative of the current health of a user, the state of a user's kidney, current treatment recommended to a user, and/or physiological parameters of a user when experiencing different stages of kidney disease) stored in user profile 118 for each user.

As mentioned, sensor electronics module 204 may be in communication with a medical device 208. Medical device 208 may be a passive device in some example embodiments of the disclosure. For example, medical device 208 may be an insulin pump for administering insulin to a user. For a variety of reasons, it may be desirable for such an insulin pump to receive and track potassium and/or glucose values transmitted from continuous analyte monitoring system 104, where continuous analyte sensor 202 is configured to measure potassium and/or glucose.

Further, as mentioned, sensor electronics module 204 may also be in communication with other non-analyte sensors 206. Non-analyte sensors 206 may include, but are not limited to, an altimeter sensor, an accelerometer sensor, a temperature sensor, a respiration rate sensor, a sweat sensor, etc. Non-analyte sensors 206 may also include monitors such as heart rate monitors, ECG monitors, blood pressure monitors, pulse oximeters, caloric intake, and medicament delivery devices. Non-analyte sensors 206 may also include data systems for measuring non-patient specific phenomena such as time, ambient pressure, or ambient temperature which could include an atmospheric pressure sensor, an external air temperature sensor or a clock, timer, or other time measure of when the sensor was first inserted or a measure of sensor life remaining compared to insertion time could be used as calibration or other data inputs for an algorithmic model. One or more of these non-analyte sensors 206 may provide data to decision support engine 114 described further below. In some aspects, a user may manually provide some of the data for processing by training server system 140 and/or decision support engine 114 of FIG. 1.

In certain embodiments, the non-analyte sensors 206 may be combined in any other configuration, such as, for example, combined with one or more continuous analyte sensors 202. As an illustrative example, a non-analyte sensor, e.g., a temperature sensor, may be combined with a continuous analyte sensor 202 configured to measure potassium to form a potassium/temperature sensor used to transmit sensor data to sensor electronics module 204 using common communication circuitry. As another illustrative example, a non-analyte sensor, e.g., a temperature sensor, may be combined with a multi-analyte sensor 202 configured to measure potassium and glucose to form a potassium/glucose/temperature sensor used to transmit sensor data to the sensor electronics module 204 using common communication circuitry.

In certain embodiments, a wireless access point (WAP) may be used to couple one or more of continuous analyte monitoring system 104, the plurality of display devices, medical device(s) 208, and/or non-analyte sensor(s) 206 to one another. For example, WAP 138 may provide Wi-Fi and/or cellular connectivity among these devices. Near Field Communication (NFC) and or Bluetooth may also be used among devices depicted in diagram 200 of FIG. 2.

FIG. 3 illustrates a diagram 300 of example inputs and example metrics that are calculated based on the inputs for use by the decision support system of FIG. 1, according to some embodiments disclosed herein. In particular, FIG. 3 provides a more detailed illustration of example inputs and example metrics introduced in FIG. 1.

FIG. 3 shows example inputs 128 on the left, application 106 and decision support engine 114 including DAM 116 in the middle, and metrics 130 on the right. In certain embodiments, each one of metrics 130 may correspond to one or more values, e.g., discrete numerical values, ranges, or qualitative values (high/medium/low, stable/unstable, etc.). Application 106 obtains inputs 128 through one or more channels (e.g., manual user input, sensors/monitors, other applications executing on display device 107, EMRs, etc.). As mentioned previously, in certain embodiments, inputs 128 may be processed by DAM 116 and/or decision support engine 114 to output metrics 130. Inputs and metrics 130 may be used by decision support engine 114 to provide decision support to the user. For example, inputs 128 and metrics 130 may be used by training server system 140 to train and deploy one or more machine learning models for use by decision support engine 114 for providing decision support around kidney disease.

In certain embodiments, starting with inputs 128, food consumption information may include information about one or more of meals, snacks, and/or beverages, such as one or more of the size, content (milligrams (mg) of potassium, carbohydrate, fat, protein, etc.), sequence of consumption, and time of consumption. In certain embodiments, food consumption may be provided by a user through manual entry, by providing a photograph through an application that is configured to recognize food types and quantities, and/or by scanning a bar code or menu. In various examples, meal size may be manually entered as one or more of calories, quantity (e.g., “three cookies”), menu items (e.g., “Royale with Cheese”), and/or food exchanges (e.g., 1 fruit, 1 dairy). In some examples, meal information may be received via a convenient user interface provided by application 106. In some examples, meal information may be provided via one or more other applications synchronized with application 106, such as one or more other mobile health applications executed by display device 107. In such examples, the synchronized applications may include, e.g., an electronic food diary application or photograph application.

In certain embodiments, food consumption information entered by a user may relate to potassium consumed by the user. Potassium for consumption may include any natural or designed food or beverage that contains potassium, such as apricot juice, avocadoes, beans, bananas, or potatoes, for example. Food consumption information entered by a user may also be related to other analytes, including any of the other analytes described herein.

In certain embodiments, exercise information is also provided as an input. Exercise information may be any information surrounding activities, such as activities requiring physical exertion by the user. For example, exercise information may range from information related to low intensity (e.g., walking a few steps) and high intensity (e.g., five mile run) physical exertion. In certain embodiments, exercise information may be provided, for example, by an accelerometer sensor on a wearable device such as a watch, fitness tracker, and/or patch. In certain embodiments, exercise information may also be provided through manual user input and/or through a surrogate sensor and prediction algorithm measuring changes to heart rate (or other cardiac metrics). When predicting that a user is exercising based on his/her sensor data, the user may be asked to confirm if exercise is occurring, what type of exercise, and or the level of strenuous exertion being used during the exercise over a specific period. This data may be used to train the system to learn about the user's exercise patterns to reduce the need for confirmation questions as time progresses. Other analytes and sensor data may also be included in this training set, including analytes and other measured elements described herein including temporal elements such as time and day.

In certain embodiments, user statistics, such as one or more of age, height, weight, BMI, body composition (e.g., % body fat), stature, build, or other information may also be provided as an input. In certain embodiments, user statistics are provided through a user interface, by interfacing with an electronic source such as an electronic medical record, and/or from measurement devices. In certain embodiments, the measurement devices include one or more of a wireless, e.g., Bluetooth-enabled, weight scale and/or camera, which may, for example, communicate with the display device 107 to provide user data.

In certain embodiments, treatment/medication information is also provided as an input. Medication information may include information about the type, dosage, and/or timing of when one or more medications are to be taken by the user. As mentioned herein, the medication information may include information about one or more diuretics, one or more drugs known to reduce potassium levels, one or more drugs known to damage the kidney, one or more drugs known to control the complications of acute or chronic kidney disease that are prescribed to the user, and/or one or more medications for treating one or more symptoms of acute or chronic kidney disease, hyperkalemia, hypokalemia, diabetes, and/or other conditions and diseases the user may have. Treatment information may include information regarding different lifestyle habits, surgical procedures, and/or other non-invasive procedures recommended by the user's physician. For example, the user's physician may recommend a user increase/decrease their potassium intake, exercise for a minimum of thirty minutes a day, or increase an insulin dosage or other medication to maintain, and/or improve, kidney health, reduce hyper- and/or hypokalemic episodes, etc. As another example, a healthcare professional may recommend that a user engage in at-home dialysis treatment and/or dialysis treatment at a clinic. Dialysis is a treatment for kidney failure that rids the body of unwanted toxins, waste products, and excess fluids by filtering a user's blood. Users with end stage renal disease (ESRD) (e.g., CKD stage 5) may be prescribed dialysis treatment to supplement and/or replace filtering generally performed by the kidney, given dialysis helps to keep the potassium, phosphorus, and sodium levels in a patient's body balanced. Dialysis treatment may be hemodialysis or peritoneal dialysis. In hemodialysis, blood is pumped out of a user's body to an artificial kidney machine, and returned to the body by tubes that connect the user to the machine. In peritoneal dialysis, the inside lining of the user's abdomen acts as a natural filter. As such, information about dialysis treatment for the user may be included in the treatment/medication information. In certain embodiments, treatment/medication information may be provided through manual user input.

In certain embodiments, analyte sensor data may also be provided as input, for example, through continuous analyte monitoring system 104. In certain embodiments, analyte sensor data may include potassium data (e.g., a user's potassium values) measured by at least a CPM (or multi-analyte sensor configured to measure at least potassium) that is a part of continuous analyte monitoring system 104. In certain embodiments, analyte sensor data may include glucose data measured by at least a glucose sensor (or multi-analyte sensor configured to measure at least glucose) that is a part of continuous analyte monitoring system 104. In certain embodiments, analyte sensor data may include creatinine data measured by at least a creatinine sensor (or multi-analyte sensor configured to measure at least creatinine) that is a part of continuous analyte monitoring system 104. In certain embodiments, analyte sensor data may include BUN data measured by at least a BUN sensor (or multi-analyte sensor configured to measure at least BUN) that is a part of continuous analyte monitoring system 104. In certain embodiments, analyte sensor data may include C-peptide data measured by at least a C-peptide sensor (or multi-analyte sensor configured to measure at least C-Peptide) that is a part of continuous analyte monitoring system 104. In certain embodiments, analyte sensor data may include cystatin C data measured by at least a cystatin C sensor (or multi-analyte sensor configured to measure at least cystatin C) that is a part of continuous analyte monitoring system 104. In certain embodiments, analyte sensor data may include lactate data measured by at least a lactate sensor (or multi-analyte sensor configured to measure at least lactate) that is a part of continuous analyte monitoring system 104. In certain embodiments, analyte sensor data may include inulin data measured by at least an inulin sensor (or multi-analyte sensor configured to measure at least inulin) that is a part of continuous analyte monitoring system 104. In certain embodiments, analyte sensor data may include dextran data measured by at least a dextran sensor (or multi-analyte sensor configured to measure at least dextran) that is a part of continuous analyte monitoring system 104. In certain embodiments, analyte sensor data may include saccharin data measured by at least a saccharin sensor (or multi-analyte sensor configured to measure at least saccharin) that is a part of continuous analyte monitoring system 104. In certain embodiments, analyte sensor data may include iothalamate data measured by at least an iothalamate sensor (or multi-analyte sensor configured to measure at least iothalamate) that is a part of continuous analyte monitoring system 104. In certain embodiments, analyte sensor data may include iohexol data measured by at least an iohexol sensor (or multi-analyte sensor configured to measure at least iohexol) that is a part of continuous analyte monitoring system 104. In certain embodiments, analyte sensor data may include 125I-iothanalamate data measured by at least a 125I-iothanalamate sensor (or multi-analyte sensor configured to measure at least 125I-iothanalamate) that is a part of continuous analyte monitoring system 104. In certain embodiments, analyte sensor data may include 51Cr-EDTA data measured by at least a 51Cr-EDTA sensor (or multi-analyte sensor configured to measure at least 51Cr-EDTA) that is a part of continuous analyte monitoring system 104. In certain embodiments, analyte sensor data may include asparagusic acid data measured by at least an asparagusic acid sensor (or multi-analyte sensor configured to measure at least asparagusic acid) that is a part of continuous analyte monitoring system 104. In certain embodiments, analyte sensor data may include polyfructosan data measured by at least a polyfructosan sensor (or multi-analyte sensor configured to measure at least polyfructosan) that is a part of continuous analyte monitoring system 104. In certain embodiments, analyte sensor data may include betanin data measured by at least a betanin sensor (or multi-analyte sensor configured to measure at least betanin) that is a part of continuous analyte monitoring system 104.

In certain embodiments, input may also be received from one or more non-analyte sensors, such as non-analyte sensors 206 described with respect to FIG. 2. Input from such non-analyte sensors 206 may include information related to a heart rate, heart rate variability (e.g., the variance in time between the beats of the heart), ECG data, a respiration rate, oxygen saturation, a blood pressure, or a body temperature (e.g. to detect illness, physical activity, etc.) of a user. In certain embodiments, electromagnetic sensors may also detect low-power radio frequency (RF) fields emitted from objects or tools touching or near the object, which may provide information about user activity or location.

In certain embodiments, input received from non-analyte sensors may include input relating to a user's insulin delivery. In particular, input related to the user's insulin delivery may be received, via a wireless connection on a smart pen, via user input, and/or from an insulin pump. Insulin delivery information may include one or more of insulin volume, time of delivery, etc. Other parameters, such as insulin action time or duration of insulin action, may also be received as inputs.

In certain embodiments, time may also be provided as an input, such as time of day or time from a real-time clock. For example, in certain embodiments, input analyte data may be timestamped to indicate a date and time when the analyte measurement was taken for the user.

User input of any of the above-mentioned inputs 128 may be through a user interface, such a user interface of display device 107 of FIG. 1.

As described above, in certain embodiments, DAM 116 and/or decision support engine (e.g., using one or more trained models) determines or computes the user's metrics 130 based on inputs 128. An example list of metrics 130 is shown in FIG. 3.

In certain embodiments, potassium levels may be determined from sensor data (e.g., potassium measurements obtained from a CPM of continuous analyte monitoring system 104, sweat sensor configured to measure potassium in sweat, where the sweat sensor may be one of non-analyte sensor(s) 206). For example, potassium levels refer to time-stamped potassium measurements or values that are continuously generated and stored over time.

In certain embodiments, a potassium baseline may be determined from sensor data (e.g., potassium measurements obtained from a continuous lactate sensor of continuous analyte monitoring system 104). A potassium baseline represents a user's normal (e.g., average) potassium levels during periods where significant fluctuations in potassium production is typically not expected. A user's baseline potassium is generally expected to remain constant over time, unless challenged through an action such as the consumption of potassium or potassium rich foods, during exercise, or changed as a result of declining kidney health or kidney dysfunction.

Each user may have a different potassium baseline. In certain embodiments, a user's potassium baseline may be determined by calculating an average of potassium levels of the user over a specified amount of time where significant fluctuations are not expected. For example, the baseline potassium for a user may be determined over a period of time when the user is sleeping, sitting in a chair, or other periods of time where the user is sedentary and not consuming food or medication which would reduce or increase potassium levels (e.g., where no external conditions exist that would affect the potassium baseline exist). In certain embodiments, DAM 116 may continuously calculate a potassium baseline, time-stamp the calculated potassium baseline, and store the corresponding information in the user's profile 118. In such embodiments, the potassium baseline may be determined based on average potassium levels through all of a user's daily activities.

In certain other embodiments, DAM 116 may use potassium levels measured over a period of time where the user is, at least for a subset of the period of time, engaging in exercise and/or consuming potassium and/or an external condition exists that would affect the potassium baseline. In such embodiments, DAM 116 may, in some examples, first identify which measured potassium values are not to be used for calculating the potassium baseline by identifying which potassium values have been affected by an external event, such as the consumption of food, exercise, medication, or other perturbation that would disrupt the capture of a potassium baseline measurement. DAM 116 may then exclude such measurements when calculating the potassium baseline of the user. In some other examples, DAM 116 may calculate the potassium baseline by first determining a percentage of the number of potassium values measured during this time period that represent the lowest lactate values measured. DAM 116 may then take an average of such potassium values to determine the potassium baseline level.

In certain embodiments, an absolute maximum potassium level may be determined from sensor data (e.g., potassium measurements obtained from a continuous CPM of continuous analyte monitoring system 104), health/sickness metrics (e.g., described in more detail below), and/or disease stage metrics (e.g., described in more detail below). The absolute maximum potassium level represents a user's maximum potassium level determined to be safe over a period of time (e.g., hourly, weekly, daily, etc.). In certain embodiments, the absolute maximum potassium level may be consistent across all users (e.g., set to 5.5 mmol/L based on current medical guidelines). In certain other embodiments, each patient may have a different absolute maximum potassium level. For example, an absolute maximum potassium level may be lower for a user diagnosed with stage 1 CKD (e.g., normal or high GFR (GFR>90 mL/min)) than a user diagnosed with stage 5 end stage CKD (e.g., GFR<15 mL/min) who also has hyperkalemia. In certain embodiments, the absolute maximum potassium level per patient may change over time. For example, a user may be initially assigned an absolute maximum potassium level based on clinical input. This assigned absolute maximum potassium level may be adjusted over time based on other sensor data, disease stages, comorbidities, etc. for the patient.

For example, a user's absolute maximum potassium level may vary over time as a user's kidney function, kidney disease, and/or one or more other diseases progress and/or improve. In certain embodiments, a first absolute maximum potassium level may be determined for periods of time where no external conditions exist that would affect the potassium level, and a second absolute maximum potassium level may be determined for periods of time where external conditions do exist that would affect the potassium level (e.g., during periods of time when the user is consuming potassium, exercising, taking medication that affects potassium levels, etc.).

In certain embodiments, an absolute minimum potassium level may be determined from sensor data (e.g., potassium measurements obtained from a continuous lactate sensor of continuous analyte monitoring system 104), health/sickness metrics (e.g., described in more detail below), and/or disease stage metrics (e.g., described in more detail below). The absolute minimum potassium level represents a user's minimum potassium level determined to be safe over a period of time (e.g., hourly, weekly, daily, etc.). In certain embodiments, the absolute minimum potassium level may be consistent across all users (e.g., set based on current medical guidelines). In certain other embodiments, each user may have a different absolute minimum potassium level. For example, an absolute minimum potassium level may be lower for a user diagnosed with stage 1 CKD (e.g., normal or high GFR (GFR>90 mL/min)) than a user diagnosed with stage 5 end stage CKD (e.g., GFR<15 mL/min) who also has hypokalemia. In certain embodiments, the absolute minimum potassium level per patient may change over time. For example, a user may be initially assigned an absolute minimum potassium level based on clinical input. This assigned absolute minimum potassium level may be adjusted over time based on other sensor data, disease stages, comorbidities, etc. for the patient.

For example, a user's absolute minimum potassium level may vary over time as a user's kidney function, kidney disease, and/or one or more other diseases progress and/or improve. In certain embodiments, a first absolute minimum potassium level may be determined for periods of time where no external conditions exist that would affect the potassium level, and a second absolute minimum potassium level may be determined for periods of time where external conditions do exist that would affect the potassium level (e.g., during periods of time when the user is consuming potassium, exercising, taking medication that affects potassium levels, etc.).

In certain embodiments, potassium thresholds other than an absolute maximum and/or minimum potassium level of a user may be determined from sensor data (e.g., potassium measurements obtained from a continuous lactate sensor of continuous analyte monitoring system 104), health/sickness metrics (e.g., described in more detail below), and/or disease stage metrics (e.g., described in more detail below). Such potassium thresholds may represent, e.g., the maximum or minimum potassium levels determined to be safe during certain activities, which may vary across different activities. For example, because exercise is known to affect potassium levels, the maximum and/or minimum potassium thresholds for a user during exercise may be different than maximum and/or minimum potassium thresholds for the user during other activities.

In certain embodiments, potassium level rates of change may be determined from sensor data (e.g., potassium measurements obtained from a CPM of continuous analyte monitoring system 104 over time). For example, a potassium level rate of change refers to a rate that indicates how one or more time-stamped potassium measurements or values change in relation to one or more other time-stamped potassium measurements or values. Potassium level rates of change may be determined over one or more seconds, minutes, hours, days, etc.

In certain embodiments, determined potassium level rates of change may be marked as “increasing rapidly” or “decreasing rapidly”. As used herein, “rapidly” may describe potassium level rates of change that are clinically significant and pointing towards a trend of the potassium levels likely breaching the absolute maximum potassium level or the absolute minimum potassium level within a defined period of time. In other words, a predictive trend (e.g., produced by decision support engine 114 using one or more trained models) may, in some cases, indicate that a patient is likely to hit, for example, the absolute maximum potassium level within a specified time period (e.g., one or two hours) based on the determined potassium level rate of change. Accordingly, such a potassium level rate of change may be marked as “increasing rapidly”. Similarly, a predictive trend (e.g., produced by decision support engine 114 using one or more trained models) may, in some cases, indicate that a patient is likely to hit the absolute minimum potassium level within a specified time period (e.g., one or two hours) based on the potassium level rate of change determined. Accordingly, such a potassium level rate of change may be marked as “decreasing rapidly”.

In certain embodiments, potassium baseline rates of change may be determined from potassium baselines determined for a user over time. For example, a potassium baseline rate of change refers to a rate that indicates how one or more time-stamped potassium baselines for a user change in relation to one or more other time-stamped potassium baselines for the same user. Potassium baseline rates of change may be determined over one or more seconds, minutes, hours, days, etc.

In certain embodiments, a potassium clearance rate may be determined from sensor data (e.g., potassium measurements obtained from a CPM of continuous analyte monitoring system 104) following the consumption of a known, or estimated, amount of potassium. Potassium clearance rates analyzed over time may be indicative of kidney function. In particular, the slope of a curve of potassium clearance during a first time period (e.g., after consuming a known amount of potassium) compared to the slope of a curve of potassium clearance during a second time period (e.g., after consuming the same amount of potassium) may be indicative of a kidney's ability to function, and more particularly, to maintain potassium homeostasis (e.g., a potassium clearance rate may be slower when a user's kidney is impaired than when a user's kidney is healthy).

In certain embodiments, the potassium clearance rate may be determined by calculating a slope between a potassium value (e.g., during a period of increased potassium levels) at to and the user's potassium baseline reached at t₁. In certain embodiments, a potassium clearance rate may be calculated over time until the increased potassium levels of the user reach some value relative to the user's potassium baseline (e.g., % of a user's potassium baseline). Potassium clearance rates calculated over time may be time-stamped and stored in the user's profile 118.

In certain embodiments, a rate of increase in potassium levels may be determined from sensor data (e.g., potassium measurements obtained from a CPM of continuous analyte monitoring system 104) following the consumption of potassium (e.g., a potassium-containing food). Rates of increase in potassium levels, as analyzed over time, may be indicative of kidney function. For example, a user may exhibit more rapid increases in potassium levels if suffering from some kidney function impairment, as the kidney would lag in clearing the potassium.

In certain embodiments, a standard deviation of potassium levels (not shown) may be determined from sensor data. In some examples, a standard deviation of potassium levels may be determined based on the variability of potassium levels as compared to an average potassium level over one or more time periods.

In certain embodiments, potassium trends may be determined based on potassium levels over certain periods of time. In certain embodiments, potassium trends may be determined based on potassium baselines over certain periods of time. In certain embodiments, potassium trends may be determined based on absolute potassium level minimums over certain periods of time. In certain embodiments, potassium trends may be determined based on absolute maximum potassium levels over certain periods of time. In certain embodiments, potassium trends may be determined based on potassium level rates of change over certain periods of time. In certain embodiments, potassium trends may be determined based on potassium baseline rates of change over certain periods of time. In certain embodiments, potassium trends may be determined based on calculated potassium clearance rates over certain periods of time.

In certain embodiments, glucose levels may be determined from sensor data (e.g., blood glucose measurements obtained from a continuous lactate sensor of continuous analyte monitoring system 104).

In certain embodiments, glucose level rates of change may be determined from sensor data (e.g., glucose measurements obtained from a continuous glucose monitor (CGM) of continuous analyte monitoring system 104 over time). For example, a glucose level rate of change refers to a rate that indicates how one or more time-stamped glucose measurements or values change in relation to one or more other time-stamped glucose measurements or values. Glucose level rates of change may be determined over one or more seconds, minutes, hours, days, etc.

In certain embodiments, a blood glucose trend may be determined based on glucose levels over a certain period of time. In certain embodiments, glucose trends may be determined based on glucose level rates of change over certain periods of time. In certain embodiments, glucose trends may be determined based on one or more glucose metrics and/or inputs over certain periods of time.

In certain embodiments, glycemic variability may be determined from sensor data (e.g., glucose measurements obtained from a continuous glucose monitor (CGM) of continuous analyte monitoring system 104 over time). For example, glycemic variability refers to a standard deviation of glucose levels over a period of time. Glycemic variability may be determined over one or more minutes, hours, days, etc.

In certain embodiments, a glucose clearance rate may be determined from sensor data (e.g., glucose levels obtained from a continuous glucose sensor of continuous analyte monitoring system 104) following the consumption of a known, or estimated, amount of glucose. Glucose clearance rates analyzed over time may be indicative of glucose homeostasis. In particular, the slope of a curve of glucose clearance during a first time period (e.g., after consuming a known amount of glucose) compared to the slope of a curve of glucose clearance during a second time period (e.g., after consuming the same amount of glucose) may be indicative of a kidney's ability to function, and more particularly, to maintain glucose homeostasis (e.g., a glucose clearance rate may be slower when a user's kidney is impaired than when a user's kidney is healthy).

In certain embodiments, the glucose clearance rate may be determined by calculating a slope between an initial high glucose value (e.g., highest glucose level during a period of 20-30 minutes after the consumption of glucose) at to and a subsequent low glucose value at t₁. The low glucose value (G_{L}) may be determined based on a user's initial high glucose value (G_{H}) and baseline glucose value (G_{B}) before the consumption of glucose. In certain embodiments, G_{L} can be a glucose value between G_{H} and G_{B}, e.g., G_{L}=G_{B}+K*(G_{H}−G_{B})/2, where K can be a percentage representing by how much a user's glucose level returned to the user's baseline value. When K equals zero, the low glucose value equals the baseline glucose value. When K equals 0.5, the low glucose value equals the mean glucose value between the initial glucose value and the baseline glucose value.

In certain embodiments, the glucose clearance rate may be determined over one or more periods of time after the consumption of glucose. The glucose clearance rate may be calculated for each time period to represent the dynamics of glucose clearance rate after the consumption of glucose. These glucose clearance rates calculated over time may be time-stamped and stored in the user's profile 118. Certain metrics may be derived from the time-stamped glucose clearance rates, such as mean, median, standard deviation, percentile, etc. In certain embodiments, a user with kidney disease may have impaired kidney function to metabolize insulin and the time passed from the initial high glucose value to the low glucose value may be indicative of a kidney's ability to function. The time passed from the initial high glucose value to the low glucose value and the glucose clearance rates may be time-stamped and stored in the user's profile 118.

In certain embodiments, insulin sensitivity may be determined using historical data, real-time data, or a combination thereof, and may, for example, be based upon one or more inputs 128, such as one or more of food consumption information, continuous analyte sensor data, non-analyte sensor data (e.g., insulin delivery information from an insulin device), etc. Insulin sensitivity refers to how responsive a user's cells are to insulin. Improving insulin sensitivity for a user may help to reduce insulin resistance in the user.

In certain embodiments, insulin on board may be determined using non-analyte sensor data input (e.g., insulin delivery information) and/or known or learned (e.g. from user data) insulin time action profiles, which may account for both basal metabolic rate (e.g., update of insulin to maintain operation of the body) and insulin usage driven by activity or food consumption.

In certain embodiments, an insulin clearance rate may be determined using historical data, real-time data, or a combination thereof, e.g., by calculating a slope between an initial insulin value (e.g., during a period of increased insulin levels) at to and a final insulin value of the user at t₁.

In certain embodiments, albumin levels may be determined from sensor data (e.g., creatinine measurements obtained from continuous analyte monitoring system 104).

In certain embodiments, an absolute maximum albumin level may be determined from sensor data (e.g., albumin measurements obtained from a continuous albumin sensor of continuous analyte monitoring system 104), health/sickness metrics (e.g., described in more detail below), and/or disease stage metrics (e.g., described in more detail below). The absolute maximum albumin level represents a user's maximum creatinine level determined to be safe over a period of time (e.g., hourly, weekly, daily, etc.). Each user may have a different absolute maximum albumin level. A user's absolute maximum albumin level may vary over time as a user's kidney function, kidney disease, and/or one or more other diseases progress and/or improve.

In certain embodiments, albumin level rates of change may be determined from sensor data (e.g., albumin measurements obtained from an albumin sensor of continuous analyte monitoring system 104 over time). For example, an albumin level rate of change refers to a rate that describes how one or more time-stamped albumin measurements or values change in relation to one or more other time-stamped albumin measurements or values. Albumin level rates of change may be determined over one or more seconds, minutes, hours, days, etc. In certain embodiments, average albumin levels may be calculated for determining rates of change of the calculated average albumin levels of the user.

In certain embodiments, albumin trends may be determined based on albumin levels over certain periods of time.

In certain embodiments, creatinine levels may be determined from sensor data (e.g., creatinine measurements obtained from continuous analyte monitoring system 104).

In certain embodiments, an absolute maximum creatinine level may be determined from sensor data (e.g., creatinine measurements obtained from a continuous creatinine sensor of continuous analyte monitoring system 104), health/sickness metrics (e.g., described in more detail below), and/or disease stage metrics (e.g., described in more detail below). The absolute maximum creatinine level represents a user's maximum creatinine level determined to be unsafe over a period of time (e.g., hourly, weekly, daily, etc.). Each user may have a different absolute maximum creatinine level. A user's absolute maximum creatinine level may vary over time as a user's kidney function, kidney disease, and/or one or more other diseases progress and/or improve.

In certain embodiments, creatinine level rates of change may be determined from sensor data (e.g., creatinine measurements obtained from a creatinine sensor of continuous analyte monitoring system 104 over time), since rates of creatinine increase and/or removal may be impaired in a user with kidney disease. For example, a creatinine level rate of change refers to a rate that describes how one or more time-stamped creatinine measurements or values change in relation to one or more other time-stamped creatinine measurements or values. Creatinine level rates of change may be determined over one or more seconds, minutes, hours, days, etc. In certain embodiments, average creatinine levels may be calculated for determining rates of change of the calculated average creatinine levels of the user. Such measurements may be taken after user consumption of a known or unknown amount of creatinine, e.g., a creatinine supplement or red meat.

In certain embodiments, creatinine trends may be determined based on creatinine levels over certain periods of time.

In certain embodiments, urea levels may be determined from sensor data (e.g., BUN measurements obtained from continuous analyte monitoring system 104). In certain embodiments, BUN trends may be determined based on BUN levels over certain periods of time.

In certain embodiments, BUN level rates of change may be determined from sensor data (e.g., BUN measurements obtained from a BUN sensor of continuous analyte monitoring system 104 over time). For example, a BUN level rate of change refers to a rate that describes how one or more time-stamped BUN measurements or values change in relation to one or more other time-stamped BUN measurements or values. BUN level rates of change may be determined over one or more seconds, minutes, hours, days, etc. In certain embodiments, average BUN levels may be calculated for determining rates of change of the calculated average BUN levels of the user.

In certain embodiments, a BUN clearance rate may be determined by calculating a slope between an initial BUN value at to (e.g., during a period of increased BUN levels) and a final BUN value of the user at to.

In certain embodiments, inulin levels may be determined from sensor data (e.g., inulin measurements obtained from continuous analyte monitoring system 104). In certain embodiments, inulin trends may be determined based on inulin levels over certain periods of time.

In certain embodiments, dextran levels may be determined from sensor data (e.g., dextran measurements obtained from continuous analyte monitoring system 104). In certain embodiments, dextran trends may be determined based on dextran levels over certain periods of time.

In certain embodiments, saccharin levels may be determined from sensor data (e.g., saccharin measurements obtained from continuous analyte monitoring system 104). In certain embodiments, saccharin trends may be determined based on saccharin levels over certain periods of time.

In certain embodiments, iothalamate levels may be determined from sensor data (e.g., iothalamate measurements obtained from continuous analyte monitoring system 104). In certain embodiments, iothalamate trends may be determined based on iothalamate levels over certain periods of time.

In certain embodiments, 125I-iothanalamate levels may be determined from sensor data (e.g., 125I-iothanalamate measurements obtained from continuous analyte monitoring system 104). In certain embodiments, 125I-iothanalamate trends may be determined based on 125I-iothanalamate levels over certain periods of time.

In certain embodiments, cystatin C levels may be determined from sensor data (e.g., cystatin C measurements obtained from continuous analyte monitoring system 104). In certain embodiments, C-peptide trends may be determined based on cystatin C levels over certain periods of time.

In certain embodiments, C-peptide levels may be determined from sensor data (e.g., C-peptide measurements obtained from continuous analyte monitoring system 104). In certain embodiments, C-peptide trends may be determined based on C-peptide levels over certain periods of time.

In certain embodiments, 51Cr-EDTA levels may be determined from sensor data (e.g., 51Cr-EDTA measurements obtained from continuous analyte monitoring system 104). In certain embodiments, 51Cr-EDTA trends may be determined based on 51Cr-EDTA levels over certain periods of time.

In certain embodiments, lactate levels may be determined from sensor data (e.g., lactate measurements obtained from continuous analyte monitoring system 104). In certain embodiments, lactate trends may be determined based on lactate levels over certain periods of time. In certain embodiments, information about lactate time in range (TIR) may also be determined based on lactate levels over time.

In certain embodiments, asparagusic acid levels may be determined from sensor data (e.g., asparagusic acid measurements obtained from continuous analyte monitoring system 104). In certain embodiments, asparagusic acid trends may be determined based on asparagusic acid levels over certain periods of time.

In certain embodiments, polyfructosan levels may be determined from sensor data (e.g., polyfructosan measurements obtained from continuous analyte monitoring system 104). In certain embodiments, polyfructosan trends may be determined based on polyfructosan levels over certain periods of time.

In certain embodiments, betanin levels may be determined from sensor data (e.g., betanin measurements obtained from continuous analyte monitoring system 104). In certain embodiments, betanin trends may be determined based on betanin levels over certain periods of time.

In certain embodiments, health and sickness metrics may be determined, for example, based on one or more of user input (e.g., pregnancy information or known sickness or disease information), from physiologic sensors (e.g., temperature), activity sensors, or a combination thereof. In certain embodiments, based on the values of the health and sickness metrics, for example, a user's state may be defined as being one or more of healthy, ill, rested, or exhausted.

In certain embodiments, disease stage metrics, such as for kidney disease, may be determined, for example, based on one or more of user input or output provided by decision support engine 114 illustrated in FIG. 1. In certain embodiments, example disease stages for kidney disease, can include AKI, stage 1 CKD with normal or high GFR (e.g., GFR>90 mL/min), stage 2 mild CKD (e.g., GFR=60-89 mL/min), stage 3A moderate CKD (e.g., GFR=45-59 mL/min), stage 3B moderate CKD (e.g., GFR=30-44 mL/min), stage 4 severe CKD (e.g., GFR=15-29 mL/min), and stage 5 end stage CKD (e.g., GFR<15 mL/min). In certain embodiments, example disease stages may be represented as a GFR value/range, severity score, and the like.

In certain embodiments, the meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first).

In certain embodiments, meal habits metrics are based on the content and the timing of a user's meals. For example, if a meal habit metric is on a scale of 0 to 1, the better/healthier meals the user eats the higher the meal habit metric of the user will be to 1, in an example. Also, the more the user's food consumption adheres to a certain time schedule or a recommended diet, the closer their meal habit metric will be to 1, in the example.

In certain embodiments, medication adherence (not shown) is measured by one or more metrics that are indicative of how committed the user is towards their medication regimen. In certain embodiments, medication adherence metrics are calculated based on one or more of the timing of when the user takes medication (e.g., whether the user is on time or on schedule), the type of medication (e.g., is the user taking the right type of medication), and the dosage of the medication (e.g., is the user taking the right dosage). In certain embodiments, medication adherence of a user may be determined in a clinical trial where medication consumption and timing of such medication consumption is monitored, through user input, and/or based on analyte data received from analyte monitoring system 104.

In certain embodiments, the activity level metric may indicate the user's level of activity. In certain embodiments, the activity level metric may be determined, for example based on input from an activity sensor or other physiologic sensors, such as non-analyte sensors 206. In certain embodiments, the activity level metric may be calculated by DAM 116 based on one or more of inputs 128, such as one or more of exercise information, non-analyte sensor data (e.g., accelerometer data), time, user input, etc. In certain embodiments, the activity level may be expressed as a step rate of the user. Activity level metrics may be time-stamped so that they can be correlated with the user's lactate levels at the same time.

In certain embodiments, exercise regimen metrics (not shown) may indicate one or more of the type of activities the user engages in, the corresponding intensity of such activities, frequency the user engages in such activities, etc. In certain embodiments, exercise regimen metrics may be calculated based on one or more of analyte sensor data input (e.g., from a lactate monitor, a glucose monitor, etc.), non-analyte sensor data input (e.g., non-analyte sensor data input from an accelerometer, a heart rate monitor, a respiration rate sensor, etc.), calendar input, user input, etc.

In certain embodiments, body temperature metrics may be calculated by DAM 116 based on inputs 128, and more specifically, non-analyte sensor data from a temperature sensor. In certain embodiments, heart rate metrics (e.g., including heart rate and heart rate variability) may be calculated by DAM 116 based on inputs 128, and more specifically, non-analyte sensor data from a heart rate sensor. In certain embodiments, respiratory metrics (not shown) may be calculated by DAM 113 based on inputs 128, and more specifically, non-analyte sensor data from a respiratory rate sensor. In certain embodiments, blood pressure metrics (e.g., including blood pressure levels and blood pressure trends) may be calculated by DAM 113 based on inputs 128, and more specifically, non-analyte sensor data from blood pressure sensor.

In certain embodiments, as described in more detail below, physiological parameters (e.g., potassium levels, potassium level rates of change, glucose levels, creatinine levels, albumin levels, heart rate, blood pressure, etc.) associated with the user may be stored as metrics 130 when a diagnosis, presence, stage (e.g., severity), or risk of kidney disease is confirmed. In certain embodiments, such physiological parameters may be analyzed over time to provide an indication of the improvement or the deterioration of a user's kidney disease. In certain embodiments, the user specific values of the physiological parameters experienced by the user may be a valuable input for training one or models designed to assess the presence and/or severity of kidney disease in a user. In certain embodiments, the user specific values of the physiological parameters experienced by the user may be used to create one or more personalized models specific to the user for more accurately predicting the presence and/or severity of kidney disease in the user.

### Example Methods and Systems for Providing Decision Support Around Kidney Disease

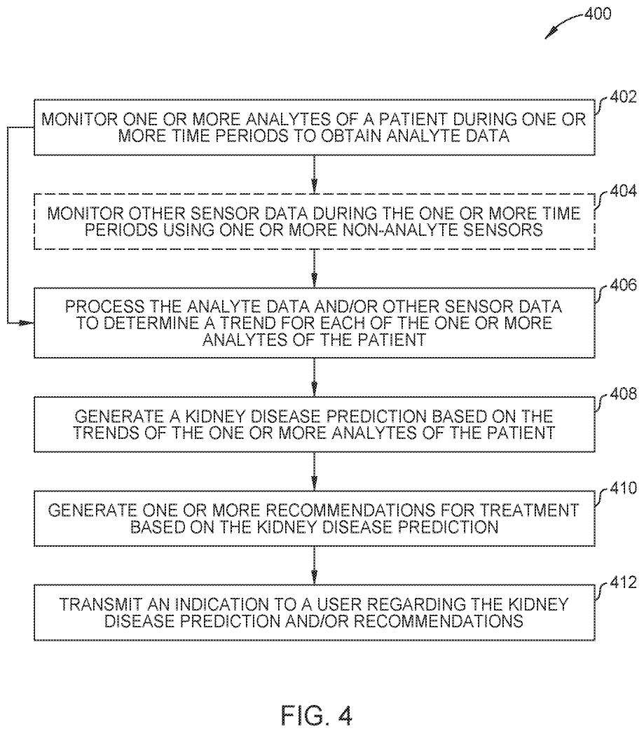

FIG. 4 is a flow diagram illustrating an example method 400 for providing decision support using a continuous analyte sensor, in accordance with certain example aspects of the present disclosure. For example, method 400 may be performed to provide decision support to a user using a continuous analyte monitoring system 104 including, at least, a continuous analyte sensor 202, as illustrated in FIGS. 1 and 2. Method 400 may provide decision support in real-time or within a specified period of time and retraining or updating of, e.g., machine learning models, based on patient input and/or diagnostics tests.

In certain embodiments, decision support engine 114 of decision support system 100 may use various algorithms or artificial intelligence (AI) models, such as machine-learning models, trained based on patient-specific data and/or population data to provide kidney disease predictions. The algorithms and/or machine-learning models may take into account one or more inputs 128 and/or metrics 130 described with respect to FIG. 3 for a patient when providing predictions related to screening, diagnosing, and staging of kidney disease.

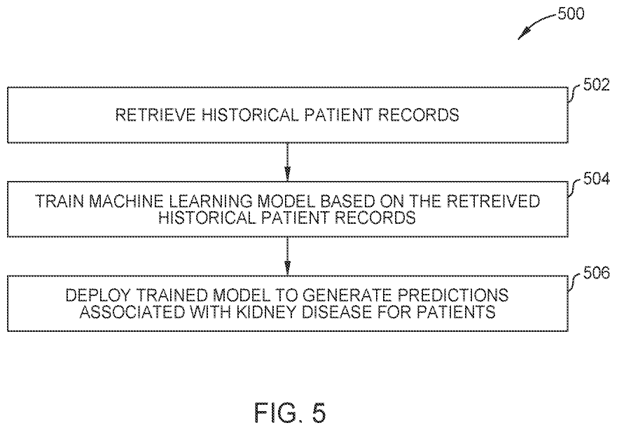

The one or more machine-learning models described herein for making such predictions may be at least initially trained using population data. A method for training the one or more machine learning models may be described in more detail below with respect to FIG. 5.

In certain embodiments, as an alternative to using machine learning models, decision support engine 114 may use rule-based models to predict the risk or likelihood of a patient experiencing kidney disease. Rule-based models involve using a set of rules for analyzing data. These rules are sometimes referred to as ‘If statements’ as they tend to follow the line of ‘If X happens then do or conclude Y’. In particular, decision support engine 114 may apply rule-statements (e.g., if, then statements) to determine the risk or likelihood of a patient developing kidney disease, experiencing kidney disease, and/or a stage of kidney disease.

Such rules may be defined and maintained by decision support engine 114 in a reference library. For example, the reference library may maintain ranges of analyte (e.g., potassium) levels and ranges of analyte level rates of change (and/or other analyte data) and/or other analyte metrics, which may be mapped to, e.g., different kidney disease risk stratifications or different stages of kidney disease. In certain embodiments, such rules may be determined based on empirical research or an analysis of historical patient records, such as the records stored in historical records database 112. In some cases, the reference library may become very granular. For example, other factors may be used in the reference library to create such “rules”. Other factors may include gender, age, diet, disease history, family disease history, body mass index (BMI), etc. Increased granularity may provide more accurate outputs.

Returning now to FIG. 4, method 400 may be performed by decision support system 100 to collect/generate data such as inputs 128 and metrics 130, including for example, analyte data, patient information, and non-analyte sensor data mentioned above, to: (1) automatically detect and classify abnormal kidney function; (2) assess the risk of kidney disease; and (3) assess the presence and stage of kidney disease. In other words, the decision support system presented herein may offer information to direct and help improve care for patients with, or at risk, of kidney disease, and more particularly, chronic kidney disease (CKD). Method 400 is described below with reference to FIGS. 1 and 2 and their components.

Generally, real-time or continuous measurements of analyte levels, rates of change, trends, clearance rates, and/or other analyte data, as measured in interstitial fluid or blood, can be used to indicate change in kidney function (e.g., either impairment or improvement of function). Such data can indicate a change in kidney function well in advance of, e.g., basal levels of creatinine as measured by glomerular filtration rate (GFR) tests, which only change once there is significant loss of kidney function, as well as other conventional kidney disease diagnostic tools such as albumin-to-creatinine ratio (ACR) tests, electrocardiograms (ECG), etc. Therefore, continuous analyte monitoring of one or more analytes, such as potassium, may provide earlier, and/or improved screening, diagnosis, prognosis, and/or staging of kidney disease as compared to conventional diagnostics.

Thus, at block 402, method 400 begins by continuously monitoring one or more analytes of a patient, such as user 102 illustrated in FIG. 1, during one or more time periods (e.g., a plurality of time periods) to obtain analyte data. The one or more analytes monitored may, in certain embodiments, include at least potassium; thus, the analyte data may at least contain potassium data. Block 402 may be performed by continuous analyte monitoring system 104 illustrated in FIGS. 1 and 2, and more specifically, continuous analyte sensor(s) 202 illustrated in FIG. 2, in certain embodiments. For example, continuous analyte monitoring system 104 may in certain embodiments comprise a continuous potassium monitor (CPM) 202 configured to measure the patient's potassium levels.

As mentioned, potassium is one of the most important minerals in the body. Potassium helps to regulate fluid balance, muscle contractions, and nerve signals. A high-potassium diet may also help to reduce blood pressure and water retention, protect against stroke, and prevent osteoporosis and kidney stones. Approximately 98% of the body's potassium is stored intracellularly, while the remaining 2% is stored extracellularly. Thus, rapid release of potassium from cells, which may occur as a result of, e.g., cell injury, cell lysis (e.g., red blood cell (RBC) lysis), and exercise, may dramatically affect extracellular potassium levels (e.g., blood potassium levels).

Generally, normal (e.g., baseline) blood potassium levels of a patient may range between 3.6 and 5.3 millimoles per liter (mmol/L). When blood potassium levels of a patient range between 5.3-6.0 mmol/L, a user may be considered to have elevated blood potassium levels which require close monitoring. When blood potassium levels are beyond the elevated range, e.g., higher than 6.0 mmol/L, the condition may be described as “hyperkalemia,” or high blood potassium levels. Hyperkalemia can increase the risk of cardiac arrhythmia episodes and even sudden death. Symptoms associated with mild hyperkalemia include muscle weakness, numbness, tingling, nausea, or other unusual feelings, while symptoms of very elevated potassium levels include heart palpitations, shortness of breath, chest pain, nausea, or vomiting. In more severe cases of hyperkalemia, patients may experience respiratory failure, sudden cardiac death, or other mortality-driven events. Conversely, when blood potassium levels are lower than normal, e.g., lower than 2.5 mmol/L, the condition may be described as “hypokalemia,” or low blood potassium levels. Low potassium levels in a patient with untreated kidney disease may lead to hypokalemia. Similar to hyperkalemia, severe hypokalemia can lead to symptoms of respiratory failure, sudden cardiac death, arrhythmias, or other mortality-driven events.

The kidneys are primarily responsible for maintaining total body potassium content/distribution by matching potassium intake with potassium excretion. Adjustments in renal potassium excretion occur over several hours; therefore, changes in extracellular potassium concentration are initially buffered by movement of potassium into or out of skeletal muscle. The regulation of potassium distribution between the intracellular and extracellular space is referred to as internal potassium balance. The most important factors regulating this movement under normal conditions are insulin and catecholamines (e.g., dopamine, epinephrine (adrenaline), and norepinephrine). In other words, the kidneys play a major role in potassium homeostasis by renal mechanisms that transport and regulate potassium secretion, reabsorption and excretion. However, when a kidney becomes damaged, or loses its ability to function, the kidney may no longer be capable of removing excess potassium; thus, potassium levels may build up in the body, e.g., causing hyperkalemia. Accordingly, potassium monitoring may prove to be useful for detecting and classifying abnormal kidney function, assessing kidney health, assessing the risk of or diagnosing or monitoring kidney disease, and/or identifying a stage of kidney disease. In certain examples, potassium monitoring may provide useful for assessing the risk of other adverse health events which may occur as a result of kidney disease.

Accordingly, in certain embodiments, a continuous potassium monitoring (CPM) sensor, e.g., CPM 202, may collect potassium measurements that can be utilized to generate potassium data including potassium baselines, potassium rates of change, potassium baseline rates of change, personalized potassium levels, average potassium levels, maximum and/or minimum potassium levels, absolute maximum and/or minimum potassium levels, standard deviation of potassium levels, potassium clearance rates, potassium trends, etc.

In certain embodiments, the potassium data includes average potassium levels of the patient. Typically, when kidney disease (e.g., CKD) progresses in a patient, the average potassium levels of the patient rise. Therefore, a CPM may be used to monitor a patient's potassium levels over a period of time, including but not limited to real-time potassium levels, potassium rates of change, and potassium clearance rates, and these measurements may be averaged over the period of time.

In some instances, a change in average potassium levels from one time period to another time period may indicate new or worsening kidney disease in a patient. For example, an average potassium level of the patient during an initial first period (e.g., a first month) of time may be compared to an average potassium level of the patient during a subsequent second period of time (e.g., a second month), whereby a difference in average potassium levels between the first time period and the second time period may suggest a change in kidney function of the patient. In such examples, an increase in average potassium levels between the first time period and the second time period may indicate new or worsening kidney disease. In another example, a patient's average potassium levels may be monitored for several periods of time to determine one or more trends (e.g., an increasing/decreasing average potassium level over several periods of time). In such examples, an increasing trend in average potassium levels may indicate worsening kidney disease.

Alternatively, in some instances, a change in average potassium levels may indicate improving or stable kidney disease. For example, circumstances where an average potassium level for a patient during a first time period is similar to an average potassium level for the patient during a second month may indicate stabilized kidney disease in the patient. In another example, wherein an average potassium level for the patient during a first time period is higher than an average potassium level for the patient during a second time period, such circumstances may indicate improving kidney disease in the patient.

In certain embodiments, in addition to average potassium levels, a standard deviation of potassium levels may be used to indicate kidney disease progression. In such embodiments, an increased variation in potassium levels, i.e., a higher standard deviation of potassium levels, may indicate worsening kidney disease in a patient, while decreased variation in potassium levels, i.e., a lower standard deviation of potassium levels, may indicate improving or stable kidney disease in the patient. In some examples, a standard deviation of potassium levels for a patient may be determined based on a variation of the patient's potassium levels as compared to the patient's average potassium level(s) over one or more time periods. In such examples, wherein a standard deviation increases from a first time period to a subsequent second time period, such circumstances may indicate new or worsening kidney disease in the patient. Alternatively, wherein a standard deviation decreases from a first time period to a subsequent second time period, such circumstances may indicate improving kidney disease in the patient. Even further, a standard deviation that is similar between a first time period and a subsequent second time period may indicate stabilized kidney disease in the patient. Together with average potassium levels, standard deviation of potassium levels may be used to refine and/or correct glomerular filtration rate (GFR) test data.

In certain embodiments, a CPM, e.g., CPM 202, may also be utilized to collect potassium measurements for generation of potassium clearance rates. To determine a potassium clearance rate, a user may consume or otherwise administer (e.g., inject) a known or estimated amount of potassium. Potassium levels, rates of change, etc., in the patient may then be monitored following consumption or administration of the potassium to determine a potassium clearance rate. In certain embodiments, the potassium clearance rate may be determined by calculating a slope between an initial potassium value (e.g., after consumption/administration of potassium) and a potassium baseline associated with the user. In certain embodiments, a potassium clearance rate may be calculated over time until the increased potassium levels of the patient reach some value relative to the patient's potassium baseline (e.g., % of a patient's potassium baseline). Generally, potassium clearance rates over time, (e.g. potassium clearance rates for one or multiple time periods), may be used to determine a change in kidney function. For example, wherein a patient's potassium clearance rate during a first time period is higher than a potassium clearance rate during a second time period, such circumstances may indicate that the patient's kidney function has declined (e.g., kidney disease in the patient is worsening).

In certain embodiments, potassium levels may be personalized in order to provide context to a patient's collected potassium measurements. Such personalized potassium levels may be utilized in conjunction with potassium level averages, standard deviations, and/or other analyte data to indicate abnormal kidney function, indicate the presence and/or progression kidney disease, and/or in certain embodiments, indicate a risk of other adverse health events which may occur as a result of kidney disease. In certain embodiments, personalization of potassium levels includes associating determined potassium levels with one or more changes (e.g., deltas) relative to a patient's potassium baseline (e.g., where a patient does not have kidney disease, is not experiencing any kidney disease-related symptoms, or participating in any activities affecting potassium) to indicate high and/or low potassium levels for that patient. For example, a patient may have a personalized “high” potassium level established as an increase of 0.3 mmol/L over baseline, and a baseline of 5.2 mmol/L, whereby when the patient's potassium levels reach 5.6 mmol/L, the patient is determined to have “high” potassium levels. In this example, another patient may not be determined to have “high” potassium levels when such patient's levels reach 5.6 mmol/L. In certain embodiments, personalization of potassium levels includes associating determined potassium levels with personalized thresholds, such as personalized thresholds for hyperkalemia and/or hypokalemia. In certain embodiments, personalization of potassium levels includes associating determined potassium levels with personalized rates of change (e.g., rapidly increasing and/or rapidly decreasing). In certain embodiments, personalization of potassium levels includes associating determined potassium levels with, e.g., signs and symptoms of hyperkalemia and/or hypokalemia (or other symptoms/conditions of kidney disease) to determine at what potassium level a patient may experience such conditions.

In certain embodiments, potassium levels may be personalized based on a patient's behavior, such as their daily activities. Generally, a patient's potassium levels rise and fall throughout the day as a result of activities performed by the patient. Such activities may include exercise, diet, posture, urine output, and the like. For example, if a patient exercises, the patient's potassium levels may rise during performance of the exercise, and then fall after performance of the exercise. On the other hand, the patient's potassium levels may be low prior to consumption of a meal, and then rise after consumption of the meal. Accordingly, potassium levels during these activities may be used in various ways to derive conclusions about the state of the user, and/or indicate the presence and/or severity of kidney disease.

In certain embodiments, potassium levels may be personalized based on non-analyte data. For example, potassium levels may be personalized by associating determined potassium levels with metrics derived from EKG signals (e.g., QRS interval, peak T wave, P wave duration, PR interval, etc.).

For example, in certain embodiments, a personalized potassium baseline may be determined based on average potassium levels throughout one or more of a patient's daily activities. In such examples, potassium levels obtained during activities which are known to affect potassium levels may be excluded from the determination of the personalized potassium baseline. Exercise is one such activity known to affect potassium levels, and thus, potassium levels during exercise may be excluded from the determination of the personalized potassium baseline. In certain embodiments, a personalized activity-specific potassium baseline may be determined based on average potassium levels of the patient during performance of such activity. In certain embodiments, potassium thresholds for certain activities may be adjusted (e.g., personalized) for a patient based on the expected change in potassium levels for the patient due to that activity. For example, because exercise is known to affect potassium levels, when a patient is exercising, different potassium thresholds may be used for deriving insight about the patient's kidney health. In certain embodiments, potassium levels during certain activities may be used to indicate presence and/or severity of kidney disease. For example, in certain embodiments, potassium levels may be correlated with urine output, and potassium levels during a time of high and/or low urine output may be used to indicate kidney dysfunction and/or change in kidney function for the patient.

Certain activities of a patient may be determined automatically through analyte monitors (e.g., potassium, glucose, lactate, etc.) and/or non-analyte monitors (e.g., HR sensor, accelerometer, etc.). Exercise is one activity which may be determined through a combination of non-potassium analyte monitors. For example, exercise may be associated with an increase in analyte levels (e.g., lactate), as well as an increase in non-analyte metrics (e.g., HR). The combination of analyte and non-analyte data may be used to determine that a patient is exercising during a time period, and as a result, potassium levels during that time period may be annotated or flagged, e.g., as “exercise potassium levels.” The annotated or flagged potassium levels may then be excluded, or used, in the above described determinations. For example, the annotated or flagged exercise potassium levels may be excluded from a personalized potassium baseline determination. In certain embodiments, other types of monitors (e.g., analyte and/or non-analyte) may be used to automatically determine daily activities upon which additional insight may be derived including, but not limited to, exercise, diet, posture, bathroom/urine output.

In certain embodiments, techniques may be introduced to account and/or correct for inaccurate potassium levels, trends, potassium variability, averages, etc. For example, potassium levels may be corrected for a period of time following insertion of a continuous potassium sensor continuous multi-analyte sensor, or other potassium-monitoring device. Due to the risk of hemolysis, potassium levels may need to be corrected following insertion of a sensor. Hemolysis occurs when injury, such as from insertion of a sensor, ruptures cells and releases the cells' contents into the plasma/serum. Since most potassium (98%) is stored intracellularly, hemolysis as a result of sensor insertion causes plasma and serum potassium levels to rise, especially in areas around the sensor insertion point. Thus, measured potassium levels may be higher upon sensor insertion as a result of hemolysis, and very high potassium levels at the time of sensor insertion of may be attributed to hemolysis. Decision support system 100 may therefore, in certain embodiments, exclude and/or correct for measured potassium levels during this period of time following insertion of a potassium sensor.

In certain embodiments, correction of measured potassium levels by decision support system 100 may be based on other analyte data, including, glucose and/or lactate data. Similar to potassium levels, lactate levels will increase with sensor insertion, and then normalize. Thus, the rapid rise and recovery of lactate levels may indicate the insertion of a sensor on the patient's body. And, because lactate levels and potassium levels can be correlated, the normalization of lactate levels may be used to determine when potassium levels have normalized. Thus, when a multi-analyte sensor (lactate and potassium) determines a corresponding rise in lactate levels and potassium levels, the rise in both lactate and potassium may be indicative of sensor insertion, and not due to systemic elevation of either potassium and/or lactate. In such examples, decision support system 100 may then exclude and/or correct for the elevated potassium levels and/or lactate levels during this period of time following sensor insertion.

In certain embodiments, corrections for sensor insertion may be based on a model, e.g., a machine learning model, used to predict the behavior of potassium levels following sensor insertion. Such a model may predict the rise and fall of measured potassium levels due to hemolysis following sensor insertion. The model may be trained to predict corrected potassium levels following insertion of a potassium sensor. Any deviations from the model for measured potassium levels following sensor insertion may also indicate kidney dysfunction. For example, in a healthy patient, potassium levels may rise by X percentage following sensor insertion, and thereafter take Y time to recover (e.g., when hemolysis is no longer effecting measured potassium).

In certain embodiments, the same device utilized to measure potassium levels, e.g., a multi-analyte sensor, or another device, may measure free hemoglobin and/or uses colorimetric measure to observe fluid abnormalities. Based on this information (e.g. free hemoglobin levels), the device, in some cases, may not report potassium levels and/or may alert the user that the potassium levels are likely inaccurate. In some cases, where an exact amount of free hemoglobin may be measured, then a correction factor may be applied to provide more accurate potassium levels for the patient

While the main analyte for measurement described herein is potassium, in certain embodiments, other analytes may be considered, alone or in combination (e.g., in combination with potassium). Such analytes may include glucose, albumin, creatinine, lactate, blood urea nitrogen (BUN), inulin, dextran, saccharin, iothalamate, iohexol, 125I-iothanalamate, c-peptide, 51Cr-EDTA, asparagusic acid, polyfructosan, and/or betanin; however, other analytes may also be considered.

In certain embodiments, using data for multiple analytes in combination, including data for the analytes mentioned above, may help to further inform the analysis around the risk, presence, and/or and staging of kidney disease (e.g., chronic kidney disease (CKD), as compared to data for a single analyte. For example, monitoring additional types of analytes in addition to potassium, as measured by continuous analyte monitoring system 104, may provide additional insight into kidney disease-related predictions as compared to insight derived from potassium alone. Such additional insight may include indications of other health conditions that may contribute to the progression of kidney disease, such as systemic inflammation, decreased systemic homeostasis, liver disease, etc.

The additional insight gained from using a combination of analytes, and not just a single analyte like potassium, may increase the accuracy of the prediction. For example, the probability of accurately predicting the risk, presence, and/or stage of kidney disease may be a function of a number of analytes measured for a patient. For example, in some examples, a probability of accurately predicting that a patient has or will likely develop kidney disease using only potassium data (in addition to other non-analyte data) may be less than a probability of accurately predicting the patient has or will likely develop kidney disease using potassium and glucose data (in addition to other non-analyte data), which may also be less than a probability of accurately predicting the patient has or will likely develop kidney disease using potassium, glucose, and creatinine data (in addition to other non-analyte data) for analysis.

Further, using a combination of analytes enables the determination of various ratios associated with the analytes (e.g., a potassium-to-urea ratio, an albumin-to-creatinine ratio, etc.), which can further inform the analysis around kidney disease. Such ratios may be determined based on measured analyte values, analyte thresholds, analyte rates of change, analyte variance, analyte clearance rates, and/or any other analyte data associated with the combination of analytes.

Accordingly, in certain embodiments described herein, analyte combinations, e.g., measured and collected by one (e.g., multianalyte) or more sensors for kidney disease-related predictions, include at least two or more of potassium, glucose, albumin, creatinine, lactate, blood urea nitrogen (BUN), inulin, dextran, saccharin, iothalamate, iohexol, 125I-iothanalamate, c-peptide, 51Cr-EDTA, asparagusic acid, polyfructosan, and/or betanin; however, other analyte combinations may also be considered.

In certain embodiments, at block 402, continuous analyte monitoring system 104 may continuously monitor glucose levels of a patient during a plurality of time periods. In certain embodiments, the measured glucose concentrations may be used in conjunction with potassium levels for determining the risk, presence, and/or and staging of kidney disease (e.g., CKD), since the kidneys also play an important role in the regulation of blood glucose, in addition to their role in potassium homeostasis. For example, the kidneys can raise blood glucose levels by generating glucose, via gluconeogenesis, and releasing the glucose into the blood. The kidneys can also lower blood glucose levels by filtering glucose from the blood. However, a majority of filtered glucose is then reabsorbed at proximal tubules of the kidneys for as an energy source. Additionally, since glucose levels normally have higher variability in the body than potassium levels (e.g., glucose levels fluctuate greater than potassium levels), and since glucose has a higher “healthy,” or normal, range than potassium, monitoring and analyzing glucose data may provide additional insight into kidney disease-related predictions as compared to insight derived from potassium alone.

In certain embodiments, the measured glucose concentrations may be used in conjunction with urine glucose measurements for determining the risk, presence, and/or staging of kidney disease. For example, the measured glucose concentration at which glucose appears in a urine glucose measurement may assist in determining a renal glucose threshold, which may be indicative of kidney health. By monitoring glucose concentrations in conjunction with urine glucose measurements to determine renal glucose threshold over time, decision support engine 114 may determine kidney health, kidney disease risk and/or kidney disease progression over time.

Glucose is a simple sugar (e.g., a monosaccharide). Glucose can be both ingested, as well as, produced in the body from protein, fat, and carbohydrates. Increasing glucose stimulates insulin release. Insulin causes the cells to take in glucose and potassium for fuel. Thus, insulin stimulates potassium and glucose uptake by cells, thereby reducing serum (e.g., extracellular) potassium and glucose levels. In some cases, where glucose levels of a patient are increased and rate(s) of change of glucose levels in the patient's body are high, excess insulin may be produced thereby causing movement of potassium intracellularly. On the other hand, where glucose levels of a patient are decreased and rate(s) of change of glucose levels in the patient's body are low, there may be less insulin secretion. Low insulin may lead to limited access of glucose and potassium by the cells; thus, extracellular glucose and potassium levels may increase.

Insulin is partially removed from circulation by the kidneys. Thus, as kidney function declines, insulin is cleared more slowly, and a release of insulin may have a more pronounced or prolonged reduction of glucose since the released insulin cannot be removed as quickly. A patient with kidney dysfunction is therefore at increased risk of hypoglycemia because in such a patient, insulin has higher activity and may cause a reduction in glucose levels below a healthy concentration.

Additionally, a patient suffering from kidney dysfunction may have a reduced ability to counteract falling glucose levels as gluconeogenesis in the kidneys may be impaired. Again, gluconeogenesis is the generation of glucose from precursor molecules (e.g., lactate, glycerol, and/or amino acids), and is performed by the liver and the kidneys. Gluconeogenesis is one mechanism for maintaining glucose homeostasis in the body, and its purpose is to prevent low blood glucose levels (i.e., hypoglycemia). As kidney function declines, however, gluconeogenesis in the kidney declines, and thus, limits the kidney's ability to react to falling blood glucose.

Further, high blood glucose (i.e., hyperglycemia) is known to accelerate the progression of kidney disease. For example, diabetic patients with lower blood glucose are noted to have a slower progression of kidney disease than diabetic patients with higher blood glucose. However, due to the increased risk of severe hypoglycemia and mortality, best practice clinical guidelines suggest that kidney disease patients should maintain a slightly higher blood glucose level than would be clinically beneficial to reduce this risk.

Since kidney dysfunction greatly impacts glucose homeostasis, kidney disease is common in patients with diabetes. In fact, diabetes patients are at higher risk of kidney disease; however, diabetes patients may miss warning signs of kidney dysfunction since glucose imbalance or changes in glucose control may be entirely, and incorrectly, attributed to their diabetes, rather than as an indication of kidney disease. In certain examples, patients with both diabetes and kidney disease may attribute all changes in glucose control to their diabetes, instead of a sign of kidney disease presence or progression.

Therefore, glucose, and by association, insulin, can be indicators of kidney function. For example, increased glucose in the bloodstream over time may cause the kidneys to filter too much blood. Over time, this extra work puts more pressure on the nephrons, which often results in the nephrons losing their vital filtering ability, thereby damaging the function of the kidney. Accordingly, the assessment of glucose levels over time may provide insight into the overall health of a patient's kidney, which may aid in determining whether the patient is suffering from kidney disease and the stage of kidney disease, or determining the likelihood of the patient developing kidney disease in the future. Thus, continuous analyte monitoring system 104 may include a continuous glucose monitor sensor (CGM), in addition to CPM 202, or a multi-analyte sensor configured to monitor both potassium and glucose, for collecting glucose data including glucose levels, time-stamped glucose levels, glucose rate(s) of change, glucose trend(s), glucose mean, glucose management indicator, glycemic variability, time in range (TIR), glucose clearance rate, minimum and maximum glucose levels, glucose autocorrelation feature, glucose set point, insulin clearance, and/or changes in glucose data.

In certain embodiments, glucose data collected by a CGM may be utilized in combination with A1C measurements to indicate the development of kidney dysfunction and/or the progression of kidney disease. A1C is a measurement of the glycation of hemoglobin found in red blood cells (RBCs), as determined from blood samples. More particularly, A1C is a percentage of glycation-modified hemoglobin based on assumed RBC half-life. A1C thus summarizes the duration of high blood glucose levels during the life of RBCs. For patients without kidney dysfunction, A1C measurements typically correlate with monitored blood glucose levels. Therefore, when A1C measurements do not correlate with monitored blood glucose levels (e.g., glucose time in range), the patient may be diagnosed with kidney dysfunction and/or CKD. Over time decreasing correlation between monitored glucose levels and A1C measurements may indicate the development of kidney disease and/or prompt further testing to confirm kidney health.

In some examples, CGM-monitored blood glucose levels may be reported as a measurement known as glucose management indicator (GMI). GMI is determined based on the mean (e.g., average) glucose levels of patient over a period of time. A decision support system, e.g., decision support system 100, can calculate GMI for a desired time period based on a patient's measured glucose levels during the time period utilizing the following relationship:

GMI (%)=3.31+0.02392*[mean glucose (mg/dl)]

Again, for patients having normal, healthy kidneys (e.g., without kidney disease), GMI will correlate with clinically measured A1C values. For patients suffering from kidney disease (e.g., CKD), however, GMI will not correlate with clinically measured A1C values, since kidney dysfunction reduces the half-life of RBCs. As a result of the shorter RBC half-life, clinically measured A1C, which is determined based on assumed RBC half-life, will be lower for kidney disease patients than the patients' actual A1C levels. Such A1C levels would otherwise correlate to higher glucose measurements in patients without CKD. Additionally, A1C levels are further affected by erythropoietin treatment, which may be prescribed to patients on dialysis due to CKD. Generally, erythropoietin treatment causes clinically measured A1C levels to be lower than the patients' actual A1C levels. Thus, A1C measurements are unreliable as indicators of actual blood glucose levels for patients suffering from kidney disease. A1C measurements are especially unreliable for stage 3B kidney disease and further advanced conditions. However, even though clinical A1C may lead to underestimation of actual blood glucose levels for kidney disease patients, GMI measurements are very accurate even for patients with kidney disease, since GMI is based on actual blood glucose levels. Therefore, as mentioned above, a patient with healthy kidneys will have corresponding GMI and clinical A1C measurements, while a patient with impaired kidney function will exhibit inconsistent measurements for GMI and clinical A1C. This discordance may thus be used as an indicator of impaired kidney function. Accordingly, GMI and clinical A1C measurements may be used to screen, diagnose, and/or stage kidney disease in conjunction with other diagnostic tools, e.g., potassium levels as measured by a CPM. In certain embodiments, decision support system 100 may thus alert a patient to a discordance between GMI and clinical A1C measurements (through user input, EMR, etc.), and recommend further investigation into potential RBC impairment.

In certain embodiments, GMI and clinical A1C measurements may also be utilized to determine the presence or likelihood of other conditions, in conjunction with kidney disease, that are associated with impacted RBC half-life (e.g., impaired hemolytic processing). For example, in addition to kidney disease, impaired hemolytic processing is associated with occurrence of oncologic processes, the presence of advanced liver disease, as well as other conditions. Thus, in certain embodiments, comparison of GMI and clinical A1C measurements may be repeated over multiple time periods (e.g., weeks, months, years), for a patient such as to passively monitor for impaired hemolytic processing, thereby indicating the presence and/or likelihood of certain conditions. In such embodiments, decision support alerts and/or recommendations may be provided to the patient, e.g., by decision support system 100, if there is a risk of impaired hemolytic processing. Further, where a patient has kidney disease, a change in hemolytic processing could indicate worsening kidney disease, or other serious conditions. Decision support recommendations for such patients may include an alert to the risk/change of impaired hemolytic processing, a risk of kidney disease or worsening kidney disease, and/or a need for further inquiry, which may comprise a trigger for additional kidney disease diagnostics as discussed below.

In certain embodiments, a glucose metric may be a minimum and/or maximum glucose level. For example, the minimum and/or maximum glucose level may be based on glucose levels over a day or a week, for example.

In certain embodiments, a glucose metric indicates a mean glucose level, which may be an average of two or more time-stamped glucose levels. In certain embodiments, a mean glucose level may be calculated based on glucose levels as well as other inputs 128, such as food consumption information, whereby corresponding glucose levels and food consumption information (e.g., with overlapping timestamps) may be used to determine mean glucose. A mean glucose level may be calculated over a period of time (e.g., one day) and compared to the mean glucose level(s) of subsequent day(s).

In certain embodiments, the glucose data collected by a CGM and monitored by decision support system 100 for kidney disease screening, diagnosis, and/or staging includes glycemic variability. Glycemic variability may generally include the standard deviation of glucose levels over a period of time, in addition to time in range (TIR) data. TIR refers to the one or more time periods in which glucose levels of a patient are within a certain desired range (e.g., healthy range). Since the kidneys' mechanisms to combat changes in glucose levels (i.e., gluconeogenesis and insulin clearance) are impaired with kidney injury and/or disease, glucose homeostasis in patients with kidney disease is disrupted. For such patients, blood glucose levels may have greater fluctuations, leading to increased glycemic variability. Thus, disrupted glucose homeostasis, as evidenced by increased glycemic variability, may be an indicator of the presence and/or severity of kidney disease. For example, wherein the glycemic variability of a patient with unknown kidney dysfunction is higher during a subsequent second time period as compared to an initial first time period, such circumstances may indicate impaired kidney function causing a disruption in the patient's glucose homeostasis. In another example, wherein the glycemic variability of a patient with known kidney dysfunction (e.g., at CKD Stage 3b) is higher during a subsequent second time period as compared to an initial first time period, such circumstances may indicate a decline in kidney function, e.g., from CKD stage 3b to CKD stage 4. For patients with kidney disease, blood glucose may have greater fluctuations due to kidney dysfunction. Greater blood glucose fluctuations result in increased glycemic variability. High glycemic variability may be due to higher and/or longer elevated glucose levels as well as lower and/or longer depressed glucose levels.

In certain embodiments, a glucose metric indicating glycemic variability may be a set point metric. For example, decision support engine 114 may determine a set point based on an estimation of the “mode” of glucose values for a patient (e.g., the glucose value that appears most often in a set of glucose values). The set point may be determined based on historical population data and/or the patient's historical glucose data, for example. Based on the calculated set point, the glucose metric may further indicate the time in range of glucose levels within a range of the set point value.

In certain embodiments, a glucose metric may demonstrate patterns or trends in glucose levels obtained at different time points (e.g., 5 minutes apart, 10 minutes apart, etc.). The glucose metric may be an autocorrelation score, demonstrating the similarities in patterns and trends between glucose levels obtained at different time points. The autocorrelation score may be a numerical value between 1.0 and 0.0, where 1.0 demonstrates the time-series glucose levels are correlated (e.g., the patterns of glucose levels obtained at different time points are very similar and/or the same) and 0.0 demonstrates the time-series glucose levels are not correlated (e.g., the patterns of glucose levels obtained at different time points are not similar and/or the same).

In certain embodiments, glucose data collected by a CGM and monitored by decision support system 100 for kidney disease screening, diagnosis, and/or staging may include glucose clearance rates. As kidney function declines, e.g., due to chronic kidney disease (CKD), a patient's glucose clearance rates typically change. Thus, decision support system 100 may, in certain embodiments, compare glucose clearance rates taken at different time periods to determine whether glucose clearance rates have changed over time. In such embodiments, decision support system 100 can monitor for glucose clearance rates by: continuously (1) utilizing historical data, including glucose levels over time; (2) continuously engaging the user in glucose tolerance challenges; and/or (3) automatically detecting glucose consumption and determining glucose clearance rates therefrom.

For example, at an initial time A, decision support system 100 may determine a first glucose clearance rate of a patient based on glucose measurements provided by a CGM. Then, at a subsequent time B, decision support system 100 may determine a second glucose clearance rate of the patient, which is decreased as compared to time A, such that at time B, blood glucose levels of the patient remain elevated (e.g., above a glucose baseline) for longer periods of time, but then also drop to decreased levels (e.g., below a glucose baseline) for longer periods of time. This change in glucose clearance rates, as noted above, may indicate the presence or progression of kidney dysfunction, and thus, kidney disease. Decision support system 100 may alert the patient to the change and recommend consultation with e.g., a healthcare provider and/or administration of additional kidney function testing. Further, decision support system 100 may incorporate the new glucose clearance rate (at time B) into projected glucose levels such as to optimize future recommendations for the patient.

In certain embodiments, glucose clearance rates may be analyzed in conjunction with or modified based on insulin data, such as insulin clearance rates, insulin on board, administered insulin, and insulin sensitivity. Such insulin data may be based on insulin measurements provided by a continuous insulin sensor, a multi-analyte sensor, or other device. In certain embodiments, insulin clearance may be determined in reference to a glucose clearance rate. For example, decision support system 100 may first determine a glucose clearance rate for a patient based on consumption of X amount of glucose by the patient. Then, decision support system 100 may determine a glucose-insulin clearance rate for the patient based on consumption of X amount of glucose and Y amount of insulin by the patient. The difference in glucose clearance rate and glucose-insulin clearance rate for the same amount (X) of glucose may reveal an insulin clearance rate for the patient.

In some cases, a patient may suffer from insulin resistance. Insulin resistance occurs when cells in the patient's muscles, fat, and liver don't respond well to insulin. Accordingly, glucose metabolism, as well as intracellular potassium movement, may be impaired. As a result, the patient's pancreas makes more insulin to help glucose and insulin enter the patient's cells.

Insulin resistance may have a different effect on glucose metabolism as compared to potassium metabolism. Further, the effect of insulin resistance on glucose metabolism and potassium metabolism may be different for different patients. In particular, a patient with a first insulin resistance may require an X dose of insulin to reduce extracellular potassium levels by Y, while another patient with a second insulin resistance may require a Z dose of insulin to reduce extracellular potassium levels by Y. For example, a diabetic patient with insulin resistance, may be at higher risk for hyperkalemia and may require higher insulin concentrations when using insulin for hyperkalemia management.

In other words, while glucose levels of a patient may affect the amount of insulin produced by the body, which in turn is expected to decrease the amount of extracellular potassium that is available to be measured by CPM 202 (e.g., which may measure potassium in the interstitial fluid), the effect of insulin resistance on potassium metabolization may cause less than an expected amount of potassium to be moved intracellularly, even in cases where glucose and/or insulin for the patient are elevated. Accordingly, understanding the effect of insulin resistance on glucose metabolism, as well as the effect of insulin resistance on potassium metabolism, for the patient may be necessary to make accurate predictions about potassium levels of the patient and better understand the true health of a patient's kidney(s). For example, in some cases, glucose levels, in addition to the patient's insulin resistance, may aid in understanding whether abnormal potassium levels for the patient are, in fact, attributed to declining health of the patient's kidneys, or some other reason, such as increased insulin resistance, where insulin resistance may be caused by heart disease, liver disease, obesity, etc.

In certain embodiments, other analyte data (e.g., C-peptide) may also be used in combination with glucose data, along with and/or instead of insulin data. For example, C-peptide measurements may be used to indicate endogenous insulin levels for determination of the production of endogenous insulin, as well as a dosage of exogenous insulin levels where a patient has insulin administered. In certain embodiments, glucose, C-peptide, insulin and/or other data (analyte and/or non-analyte) may be used to indicate: (1) levels of endogenous insulin; (2) exogenous insulin administered; (3) insulin (endogenous/exogenous) clearance rates; (4) metabolic rate of change of glucose based on insulin amount; and/or (5) glucose clearance in urine, based on estimated metabolic and insulin concentration-dependent rate.

In another example, at block 402, continuous analyte monitoring system 104 may continuously monitor creatinine levels of a patient during one or more time periods. In particular, creatinine is a waste product produced by muscles from the breakdown of a compound called creatine. Creatinine is removed from the body by the kidneys, which filter almost all of the creatinine from the blood and release the creatinine into the urine. Accordingly, creatinine levels may provide insight into kidney health and function. Thus, a patient experiencing high levels of measured extracellular potassium and is assumed to have damaged kidney function (e.g., given excess potassium is not being filtered from the body), may also be expected to be experiencing high levels of measured creatinine (e.g., given a damaged kidney would not likely be capable of removing the creatinine from the blood). Accordingly, in certain embodiments where creatinine levels are monitored in combination with potassium levels, the measured levels of creatinine may be used to assign a confidence level to measured potassium levels of the patient, where the confidence level indicates the level of certainty that the potassium levels measured for the patient reflect the patient's actual potassium levels. For example, where measured potassium levels and creatinine levels are high, a higher confidence level may be assigned to the potassium levels measured for the patient. Further, the assumption that the patient's kidney(s) are damaged may be strengthened, thereby increasing the likelihood that the patient's kidney(s) are not working properly and increasing the likelihood that the patient is, in fact, suffering from kidney disease.

In certain embodiments, creatinine clearance rates may be used in combination with mGFR testing. Creatinine clearance measurements may be useful in identifying changes in secretion and filtration functions of the kidneys, since both secreted and filtered creatinine may be measured: creatinine is secreted in the proximal tubule of the kidneys, and is further filtered through the glomerulus thereof. Thus, the difference between mGFR measurements and creatinine clearance rates for the same period may be used to determine secretion functionality of the kidneys. A change in secretion functionality can, in turn, be indicative of tubule health and the risk or presence of tubulointerstitial fibrosis.

In another example, at block 402, continuous analyte monitoring system 104 may continuously monitor albumin levels of a patient during one or more time periods for kidney disease screening, diagnosis, and/or staging. Albumin levels generally do not vary greatly throughout the day in either blood or interstitial fluid, and a normal range for albumin is 3.4 to 5.4 g/dL with a turnover period in approximately 25 days. Because of the relative stability of albumin levels in the body, any rapid changes in albumin level may indicate kidney dysfunction. Accordingly, in certain embodiments, decision support system 100 may determine albumin rates of change and/or albumin variability based on measured albumin levels. For example, an increase in variability of urinary albumin levels (e.g., a higher standard deviation) over a daily time period may be indicative of the presence and/or progression of kidney disease.

In certain embodiments, albumin measurements may be used in combination with creatinine measurements for kidney disease screening, diagnosis, and/or staging. For example, real-time or continuous measurements of creatinine levels and/or rates of change may be provided across minutes, hours, and/or days, in interstitial fluid or blood, and may be used in combination with baseline albumin levels to indicate kidney health. Because the rates of change of albumin and creatinine occur on different time scales, any abnormal changes in albumin and creatinine levels, and/or the ratio between albumin and creatinine levels, rates of change, or trends, over a given time period (e.g., 24 hours), may be used to indicate kidney dysfunction and/or a change in kidney function.

In certain embodiments, albumin measurements may be used in combination with creatinine and potassium measurements for kidney disease screening, diagnosis, and/or staging. More particularly, potassium, creatinine and albumin measurements may be used in combination to confirm variance in levels of one or more of these analytes is due to kidney disease.

In yet another example, at block 402, continuous analyte monitoring system 104 may continuously monitor urea levels (e.g., blood urea nitrogen (BUN) levels) of a patient during one or more time periods. Urea is synthesized in the liver and cleared by the kidneys. In particular, the liver produces ammonia, which contains nitrogen, after the liver breaks down proteins used by cells in the body. Nitrogen combines with other elements, such as carbon, hydrogen and oxygen, to form urea, which is a chemical waste product. The urea travels from the liver to the kidneys through the bloodstream. Healthy kidneys filter urea and remove other waste products from the blood, and the filtered waste products leave the body through urine. Accordingly, urea levels may provide insight into kidney health and function.

While decreased urea synthesis may indicate liver disease rather than kidney dysfunction, decreased urea synthesis is typically only found in end-stage liver disease, and therefore liver disease can be ruled-out as an unlikely cause of change in urea levels. In the absence of end-stage liver disease, changes in urea levels of a patient may thus reliably indicate changes in urea clearance by the kidneys. Accordingly, for users without end-stage liver disease, decision support system 100 may utilize urea levels to determine urea clearance rates, which may be utilized as an indicator of kidney disease risk, presence and/or progression. In certain embodiments, urea clearance rates may be used in conjunction with creatinine clearance rates as an alternative to mGFR measurements. For example, urea clearance and creatinine clearance may be monitored for a time period of one hour and clearance rates thereafter calculated. Such a determination may be equally effective as an mGFR measurement. However, for increased effectiveness, urea clearance and creatinine clearance can be monitored for 24 hours or more.

Additionally, in certain embodiments, reabsorption rates of urea may be used as a proxy for hydration or renal blood flow of a patient, and may thereby indicate kidney disease progression and stage.

In still further embodiments, measured levels of urea may also be used to assign a confidence level to measured potassium levels of a patient. More particularly, a patient experiencing high levels of measured extracellular potassium and is assumed to have damaged kidney function, may also be expected to be experiencing high levels of urea (e.g., given a damaged kidney would not likely be capable of filtering urea and removing other waste products from the blood). Accordingly, where measured urea levels are also high, a higher confidence level may be assigned to potassium levels measured for the patient. Further, the assumption that the patient's kidney(s) are damaged may be strengthened, thereby increasing the likelihood that kidney(s) of the patient are not working properly and increasing the likelihood that the patient is, in fact, experiencing (or is at risk of) kidney disease.

In yet another example, at block 402, continuous analyte monitoring system 104 may continuously monitor C-Peptide levels of a patient during one or more time periods. In certain embodiments, the measured levels of C-peptide may be used to determine endogenous insulin levels of a patient to provide additional context around potassium measurements gathered by the continuous analyte sensor 202.

In particular, C-peptide is a substance that is created when insulin is produced and released into the body. Because no method currently exists for measuring insulin in the body, the level of C-peptide in the blood can be measured to show how much insulin is being made by the pancreas. Often, C-peptide is measured to tell the difference between insulin the body produces and insulin that is injected into the body. Understanding insulin levels of a patient may provide insight into why measured potassium levels of a patient are displaying lower than normal or greater than normal values for the patient to better assess kidney health of the patient.

In yet another example, at block 402, continuous analyte monitoring system 104 may continuously monitor lactate levels. Lactate levels may be associated with glucose, insulin, and potassium metabolism. Lactate levels may also be used to detect consumption of food, exercise, rest, and/or stress. Therefore, the additional insights gained from lactate levels may improve analysis of other analyte data and trends by associating such analyte metrics with different body states (e.g., food consumed, exercise, rest, stress, etc.).

In certain embodiments, at block 402, continuous analyte monitoring system 104 may continuously monitor one or more analytes that may indicate impairment in either kidney filtration function or kidney secretion function in patients with kidney disease or differentiate between kidney filtration function versus kidney secretion function. For example, continuous analyte monitoring system 104 may continuously monitor one or more analytes that could be utilized as a marker for, or in conjunction with, glomerular filtration rate (GFR) tests to determine kidney filtration function. An ideal GFR marker comprises a small analyte that is retained in the vasculature and is not protein-bound, and that is freely filtered across the glomerulus of the kidneys. Such marker would not be reabsorbed, secreted, or metabolized by the kidneys, so that measured GFR thereof would be equal to the urinary clearance of the marker after its intravenous infusion into the patient. Further, the ideal GFR marker is also generally recognized as safe (GRAS), comprises a relatively high oral bioavailability, and is cleared by the kidneys without intervention of other metabolic pathways or interactions.

One example of a near-ideal GFR marker for continuous monitoring by continuous analyte monitoring system 104 to inform the analysis of kidney disease risk, presence, and/or progression is inulin. Inulin is an analyte that is typically measured in mGFR tests. Accordingly, continuous inulin measurements may be used in conjunction with, or as an alternative to, standard mGFR testing to improve mGFR results. For example, in certain embodiments, rather than subjecting a patient to a standard mGFR test, in which the patient must remain in a clinic for several hours and provide several blood samples, one or more continuous analyte sensors 202 of continuous analyte monitoring system 104 may comprise a continuous inulin sensor to continuously monitor inulin levels over a given time period for analysis by decision support system 100. In this example, patient convenience is improved, as monitoring of inulin via a continuous inulin sensor is less time intensive and intrusive than conventional mGFR testing. Additionally, utilization of a continuous inulin sensor 202 may reduce the time lost to early phase rapid decay and allow for a longer sampling period, thereby reducing any effects on mGFR measurements as caused by the time of day (during sampling), patient posture, and patient diet. Furthermore, utilization of continuous potassium measurements in combination with continuous inulin measurements for mGFR testing may improve the reliability and analysis of measured inulin clearance.

Another example of a potential marker that can be continuously monitored by continuous analyte monitoring system 104 for use with GFR testing to inform the analysis of kidney disease is dextran. High molecular weight dextran (such as 150 kDa or higher) remains in the blood and is not filtered by the kidneys. Thus, such high molecular weight dextran may be monitored to determine plasma volume, as measurements thereof may be utilized to quantify the plasma volume of distribution based on principles of dilution. On the other hand, low molecular weight dextran (such as 5 kDa) distributes into the interstitial space and is then filtered by the kidneys, but is not further metabolized or intracellularly distributed. Accordingly, low molecular weight dextran may be monitored to determine a renal filtration rate, since the plasma concentration of low molecular weight dextran decreases as a function of time and is dependent on both kidney clearance and redistribution within the vasculature and into extracellular fluid. However, dextran must be administered intravenously or subcutaneously (i.e., no oral bioavailability), which limits its usability as a GFR marker.

Yet another example of a potential GFR marker for continuous monitoring by continuous analyte monitoring system 104 to inform the analysis of kidney disease is saccharin. Saccharin is a small molecule with good oral bioavailability (˜98%), thus enabling non-clinical GFR measurements (e.g., at home). Saccharin is cleared by the kidneys without reabsorption and minimal protein binding. Saccharin has no other metabolic pathway, and so it can be monitored to indicate various kidney functions over time, thereby allowing for diagnosis and staging of kidney dysfunction over time. However, saccharin is also secreted by the kidneys, which may confound single-point GFR measurements. Despite this, saccharin may still be a reliable compound for GFR estimation based on continuous measurements.

In addition or alternatively to the GFR markers above, to provide additional insight into the analysis of kidney disease risk, presence, and/or progression, continuous analyte monitoring system 104 may monitor other GFR markers, including nonradioactive markers such as iothalamate, iohexol, and polyfructosan, as well as radioactive markers such as 125I-iothalamate, and 51Cr-EDTA.

In certain embodiments, the one or more algorithms and/or models described herein, e.g., for predicting the risk, presence, and/or progression of kidney disease, may be configured to use input from one or more sensors measuring one or more of the multiple analytes described above. Parameters and/or thresholds of such algorithms and/or models may be altered based, at least in part, on a number of analytes being measured for input to reflect the knowledge attained from each of the other analytes being measured.

In certain embodiments, in addition to continuously monitoring one or more analytes of a patient at block 402 as described above, one or more analytes may be monitored/measured via urinalysis. For example, chemical or photographic analysis may be used to analyze exogenous analytes present in urine. Exogenous analytes include analytes that are not naturally found in the body and may be effectively cleared from the body in a short time period by healthy kidneys. Though not naturally found in the human body, these analytes may still be naturally-occurring, such as asparagusic acid and betanin. Asparagusic acid is a sulfur-containing compound readily cleared from the body during the consumption of asparagus which could be detected by a point of care electrochemical assay on different aliquots of urine over time compared to a known ingestion time point. Betanin is another naturally-occurring analyte commonly found in beets. In high concentrations, betanin can turn the urine into a blood-red color, and this color change be readily observed by the naked eye or through electronic means at a point in time, when compared to a known ingestion point in time. Measuring a user's response to known ingestion may be a means of understanding kidney function clearance over time or, alternatively, may set the brightness of urine based on a concentration of urine expelled at a specific time point. This urine brightness may be compared with a normalized curve to determine the difference between the expected red color cleared and the actual red color cleared during a test period. The difference in actual and expected red color may help in determining the functional clearance rate of kidneys and kidney function.

For example, at block 402, a patient may consume a known volume of betanin and then capture the volume or concentration of betanin later found in the patient's urine. In one iteration, the patient may photograph a serving of beets before consuming the beets. Later, the patient may photograph their urine to capture the color change caused by betanin, and input such photograph(s) into decision support system 100. Decision support engine 114 may then analyze the photograph(s) of the serving of beets to determine an initial consumption of betanin, and further analyze the photograph(s) of the user's urine to determine a concentration of betanin present in the patient's urine.

Similarly, asparagusic acid comprises a heterocyclic disulfide functional group which can be chemically detected in urine. One method of chemical detection includes the utilization of a urine test strip comprising pads or reagents configured to react to certain concentration of asparagusic acid and change color. Another method of chemical detection includes the utilization of a chemical reactant in toilet water, which also changes color when exposed to asparagusic acid. Accordingly, similar to the example above, at block 402, a patient may capture photographs of a serving of asparagus before consumption thereof, and then later capture photographs of a test strip or toilet water (with chemical reactant) after urinating thereon. Such photographs may be inputted into decision support system 100 and analyzed by decision support engine 114 to determine an initial consumption of asparagusic acid as well as a concentration of asparagusic acid present in the patient's urine.

Furthermore, in certain examples, an initial amount of asparagusic acid or betanin can be consumed in the form of a capsule, pill, or drink, such that the initial amount of either analyte to be cleared is known. Thereafter, analyte clearance of the asparagusic acid or betanin can be determined based on the resulting urine concentrations. A comparison of the expected clearance rate and time of expected detection of analyte with the actual clearance rate and the actual time of detection may indicate kidney function and/or a need for further investigation into kidney function.

At optional block 404, method 400 continues by optionally monitoring non-analyte sensor data during the one or more time periods, using one or more non-analyte sensors or devices (e.g., such as non-analyte sensors 206 and/or medical device 208 of FIG. 2).

As mentioned previously, non-analyte sensors 206 and devices may include one or more of, but are not limited to, an insulin pump, a haptic sensor, an electrocardiogram (ECG) sensor or heart rate monitor, a blood pressure sensor, a sweat sensor, a respiratory sensor, a thermometer, a pulse oximeter, an impedance sensor, a peritoneal dialysis machine, a hemodialysis machine, sensors or devices provided by display device 107 (e.g., accelerometer, camera, global positioning system (GPS), heart rate monitor, etc.) or other user accessories (e.g., a smart watch), or any other sensors or devices that provide relevant information about the user. One or more of these non-analyte sensors 206 and devices may provide data to decision support engine 114 described above. In some aspects, a user, e.g., a patient, may manually input the data for processing by decision support engine 114.

Certain metrics, such as one or more of metrics 130 illustrated in FIG. 3, may be calculated using measured data from each of these additional sensors. Further, as illustrated in FIG. 3, one or more of the metrics 130 calculated from non-analyte sensor or device data may include body temperature, heart rate (including heart rate variability), respiratory rate, etc. In certain embodiments, described in more detail below, the one or more of the metrics 130 calculated from non-analyte sensor or device data may be used to further inform the analysis around kidney disease prediction.

In certain embodiments, one or more non-analyte sensors and/or other devices may be worn by a user to aid in the detection of periods of increased physical exertion by the user. Such non-analyte sensors and/or devices may include an accelerometer, an ECG sensor, a blood pressure sensor, a heart rate monitor, an impedance sensor, a dialysis machine, and the like. In certain embodiments, measured and collected data from periods of increased physical exertion and periods of sedentary activity by the user may be used to analyze, e.g., potassium levels, glucose levels, lactate levels, etc., during each of these identified periods to inform kidney disease prediction.

In certain embodiments, one or more non-analyte sensors and/or devices that may worn by a patient may include a temperature sensor. A temperature sensor may be worn to aid in correcting, e.g., measured potassium levels for predicting the risk, presence, and/or progression of kidney disease in the patient. In particular, a correlation exists between body temperature and potassium release in the human body.

For example, at higher body temperatures, the body sweats, and potassium is excreted through sweat. Accordingly, higher body temperatures may induce lower potassium levels. In some cases where measured potassium levels of a patient may appear to be lower than normal for that patient, one may conclude the lower than normal potassium levels of the patient are due to an inability to concentrate urine due to impaired renal tubule response to vasopressin (ADH), which leads to excretion of large amounts of dilute urine, including, in some cases, potassium. However, such decreased potassium levels may actually be due to excessive sweat, e.g., excessive potassium leaving the patient's body. In other words, high body temperature of a patient may affect the amount of extracellular potassium that is measured by a continuous potassium sensor, e.g., CPM 202. Accordingly, in certain aspects, secondary sensors, such as a temperature sensor, may be used to show that dynamic and sudden changes to potassium may be a result of sweating (e.g., associated with exercise, hot and/or humid weather, etc.) as compared to a negative health event.

As another example, in some cases, measured potassium levels for a patient initially experiencing hypothermia (e.g., occurs when the body loses heat faster than the body can produce heat, causing a dangerously low body temperature) may appear to be lower than normal potassium levels associated with the patient. Such decreased potassium levels may be attributed to the patient experiencing an onset of hypothermia. In particular, hypothermia may cause an initial decrease of extracellular potassium levels. Hypothermic hypokalemia is linked to an intracellular shift rather than an actual net loss. The intracellular shift is caused by a variety of factors such as enhanced functioning of Na+K+ATPase, beta-adrenergic stimulation, pH and membrane stabilization in deep hypothermia.

As hypothermia progresses in the patient, however, irreversible cell damage may occur. In particular, the body may experience a lack of enzyme functioning at cold temperatures and blocked active transport. Thus, as hypothermia progresses, measured potassium levels of the patient may increase from levels that are lower than normal to levels that are higher than normal. In other words, low body temperature of a patient may affect the amount of extracellular potassium that is measured by CPM 202 over time. Accordingly, monitoring the body temperature of a patient may help to inform measured potassium levels of the patient such that the measured potassium levels may be corrected prior to predicting kidney disease.

Additionally, in certain aspects, an external temperature probe may be used to measure the temperature immediately around (e.g., the area very close to) the patient to predict whether the patient is, in fact, experiencing hypothermia or decreased body temperatures. Further, in certain aspects, an accelerometer or a piezoelectric sensor may be used to identify whether a patient is shivering in order to determine whether a patient is experiencing decreased body temperatures that may be affecting the measured potassium levels of the patient.

In certain embodiments, one or more non-analyte sensors and/or devices that may be worn by a patient may include a blood pressure sensor. Blood pressure measurements collected from a blood pressure sensor may be used to provide additional insight into kidney health of the patient. In particular, kidney disease and high blood pressure are closely related. Typically, as blood pressure rises, kidney function declines. Thus, a patient assumed to have damaged kidney function as indicated by high levels of measured extracellular potassium (e.g., excess potassium is not being filtered from the body) may also be expected to be experiencing high blood pressure levels. Accordingly, where blood pressure levels for the patient are also high, the assumption that the patient's kidney(s) are damaged may be strengthened, thereby increasing the likelihood that kidney(s) of the patient are not working properly and increasing the likelihood that the patient does, in fact, suffer from kidney disease.

In certain embodiments, one or more non-analyte sensors and/or devices that may be worn by a patient may include an ECG sensor and/or a heart rate monitor. As is known in the art, an ECG device is a device that measures the electric activity of the heartbeat. In certain embodiments, heart rate measurements, as well as heart rate variability information, collected from an ECG sensor and/or a heart rate monitor may be used in combination with a CPM to better inform the assessment of kidney health. In particular, potassium levels of a patient measured using a CPM may be used to detect hyperkalemia or hypokalemia. ECG measurements, in combination with the CPM measurements, for a patient may be vital for providing a whole-picture assessment of the physiologic significance of hyperkalemia or hypokalemia.

At block 406, method 400 continues by processing the analyte data from the one or more time periods, and in certain embodiments, the other non-analyte sensor data, to determine at least one analyte trend or analyte rate of change of the patient. Block 406, in certain embodiments, may be performed by decision support engine 114.

As mentioned, an analyte trend or rate of change indicates the change of one or more time-stamped metrics, measurements or values of the analyte in relation to one or more other time-stamped measurements or values of the analyte. In certain embodiments, machine-learning models, described herein, used to provide kidney disease-related predictions may include one or more features not only related to analyte levels of the patient, but also analyte level trends and analyte level rates of change of the patient. For example, an example machine-learning model may include weights applied to features associated with the one or more trends or rates of change of, e.g., potassium levels. Thus, in certain embodiments, prior to use of the machine-learning model, at least one potassium level rate of change for the patient may need to be calculated for input into the model.

Further, in certain embodiments, rule-based models, described herein, used to provide kidney disease-related predictions, may include one or more rules not only related to analyte levels of the patient, but also analyte level trends and analyte level rates of change of the patient. For example, a reference library, used to define one or more rules for the rule-based models, may maintain ranges of, e.g., potassium levels and ranges of potassium level rates of change, which may be mapped to different stages of kidney disease. Thus, prior to use of the rule-based model, at least one potassium level rate of change for the patient may need to be calculated for input into the model. Additional features, such as potassium level rates of change, added to the model may, in some cases, allow for a more accurate prediction of kidney disease risk, presence, and/or progression for the patient.

At block 408, method 400 continues by generating a kidney disease prediction, which may include: (1) a likelihood that the patient is experiencing (or will experience) abnormal kidney function; (2) a risk of kidney disease; and (3) a presence and/or stage of kidney disease of the patient using: (a) the at least one analyte trend or analyte rate of change of the patient (e.g., determined at block 402); and (b) a trained model or one or more rules (e.g., rule-based models).

Different methods for generating a kidney disease prediction may be used by decision support engine 114. In particular, in certain embodiments, decision support engine 114 may use a rule-based model to provide real-time decision support for kidney disease risk assessment, diagnosis, and staging. As mentioned previously, rule-based models involve using a set of rules for analyzing data. In particular, decision support engine 114 may apply rule-statements (e.g., if, then statements) to assess the presence and severity of kidney disease in a patient, perform kidney disease risk stratification for a patient, and/or identify risks associated with a current kidney disease diagnosis of the patient.

For example, one rule may be related to an absolute maximum potassium level for the patient currently, or based on changes of the absolute maximum potassium level over time. Another rule may be related to an absolute minimum potassium level for the patient currently, or based on changes of the absolute minimum potassium level over time. Another rule may be based on changes of the potassium baselines of a patient over time. Another rule may be related to potassium level rates of change for the patient, whether such potassium level rates of change have been marked as “increasing rapidly” or “decreasing rapidly” (e.g., as described with respect to FIG. 3), or based on changes of the potassium level rates of change over time. Another rule may be related to glucose metrics, insulin metrics, creatinine metrics, BUN metrics, albumin metrics, dextran metrics, inulin metrics, saccharin metrics, iothalamate metrics, iohexol metrics, 125I-iothanalamate metrics, 51Cr-EDTA metrics, lactate metrics, asparagusic acid metrics, polyfructosan metrics, betanin metrics, and/or C-peptide metrics as described with respect to FIG. 3 or based on changes of such metrics over time.

Another rule may be related to a patient's glucose response, or lack thereof, to biochemical hypoglycemia (e.g., below 70 mg/dL), with or without being able to measure circulating insulin. Another rule may be related to whether a patient experiences acute or abrupt increases (and what that increase is) in creatinine concentrations due to acute kidney injury (AKI) (e.g., blood loss, vomiting, diarrhea, heart failure, etc.). Another rule may be related to a patient's potassium clearance rate following consumption of a known, or estimated, amount of potassium. For example, in a patient with impaired kidney function, the rate of clearance may be slower than in a healthy control subject.

Another rule may be related to the absolute maximum potassium level following consumption of a known, or estimated amount of potassium. For example, the absolute maximum potassium level following the consumption of a known, or estimated, amount of potassium may be greater in a patient with impaired kidney function than that of a healthy control subject. Another rule may be related to a correlation between the absolute maximum potassium level of a patient and the patient's potassium clearance rate. For example, increased absolute maximum potassium levels with reduced potassium clearance may be observed as kidney disease progresses in a patient. One or more other rules may be based on data from one or more non-analyte sensors in combination with measured potassium levels of the patient. Any of the above identified rules may be used in combination with each other, or one or more other rules, when using the rule-based model.

Such rules may be defined and maintained by decision support engine 114 in a reference library. For example, the reference library may maintain ranges of analyte levels and/or rates of changes which may be mapped to different severities of kidney disease. In certain embodiments, such rules may be determined based on empirical research as well as analyzing historical patient records from historical records database 112.

In certain embodiments, as an alternative to using a rule-based model, AI models, such as machine-learning models may be used to provide real-time decision support for kidney disease risk assessment, diagnosis, and staging. In certain embodiments, decision support engine 114 may deploy one or more of these machine learning models for performing screening, diagnosis, staging, and/or risk stratification of kidney disease in a patient. Risk stratification may refer to the process of assigning a health risk status to a patient, and using the risk status assigned to the patient to direct and improve care.

In particular, decision support engine 114 may obtain information from a user profile 118 associated with a patient, stored in user database 110, featurize information for the patient stored in user profile 118 into one or more features, and use these features as input into such models. Alternatively, information provided by the user profile 118 may be featurized by another entity and the features may then be provided to decision support engine 114 to be used as input into the ML models. In certain embodiments, features associated with the patient may be used as input into one or more of the models to assess the risk, presence, and/or severity of kidney disease in the patient.

In certain embodiments, features associated with the patient may be used as input into one or more of the models to risk stratify the patient to identify whether there is a high or low risk of the patient developing kidney disease (e.g., CKD). In certain embodiments, features associated with the patient may be used as input into one or more of the models to identify risks (e.g., mortality risk, risk of being diagnosed with one or more other diseases, etc.) associated with a current kidney disease diagnosis of the patient. In certain embodiments, features associated with the patient may be used as input into one or more of the models to perform any combination of the above-mentioned functions. Details associated with how one or more machine-learning models can be trained to provide real-time decision support for kidney disease risk assessment, diagnosis, and/or staging are further discussed in relation to FIG. 5.

As mentioned, in certain embodiments, non-analyte sensor data, in addition to analyte data, may be used by decision support engine 114 to generate a kidney disease prediction for a patient, at block 506. For example, data provided by an insulin pump, a haptic sensor, an electrocardiogram (ECG) sensor or heart rate monitor, a blood pressure sensor, a sweat sensor, a respiratory sensor, a thermometer, a pulse oximeter, an impedance sensor, a peritoneal dialysis machine, a hemodialysis machine, sensors or devices provided by display device 107 (e.g., accelerometer, camera, global positioning system (GPS), heart rate monitor, etc.) or other user accessories (e.g., a smart watch), or any other sensors or devices that provide relevant information about the user, may be used as input into such machine learning models and/or rule-based models to predict the risk, presence, and/or severity of kidney disease of a user.

Decision support engine 114 may use the machine learning models and/or the rule-based models to generate a kidney disease prediction based on continuous analysis of data (e.g., analyte data and, in some cases, non-analyte data) for the patient collected over one or more time periods. Analysis of data collected for the patient over various time periods may provide insight into whether the kidney health and/or a disease of the patient is improving or deteriorating. For example, a patient previously diagnosed with chronic kidney disease using the models discussed herein may continue to be constantly monitored (e.g., continuously collect for the patient) to determine whether the disease is getting worse or better, etc. As an example, comparison of analyte data (glucose levels, time-stamped glucose levels, glucose baselines, absolute maximum glucose levels, absolute minimum glucose levels, glucose level rates of change, glucose metrics (e.g., glucose set point metrics, glucose autocorrelation score, etc.), TIR, mean glucose, GMI, and/or glycemic variability) and/or other sensor data over multiple months may be indicative of the patient's disease progression. For example, decision support engine 114 may determine a patient is at risk of kidney disease if a patient's absolute minimum glucose levels begin to decrease over time, especially when the patient is asleep.

For example, decision support engine 114 may provide a likelihood the patient is experiencing abnormal kidney function or is at risk of kidney disease based on the patient's time-stamped glucose levels. For example, a user at risk of developing kidney disease may experience higher daytime glucose levels, a larger number of daytime hyperglycemic events, higher post-prandial glucose levels, lower nighttime glucose levels, and/or a larger number of nocturnal hypoglycemic events over time, which may demonstrate worsening glycemic control, and therefore, the presence of kidney disease.

In a certain embodiments, lower minimum glucose levels over time and/or higher maximum glucose levels over time may demonstrate that a patient is developing kidney disease and/or experiencing worsening kidney disease. For example, a patient with kidney disease may expect to experience a hyperglycemic spike after dinner and/or when the patient goes to sleep. If a patient develops a hyperglycemic spike after dinner and/or when the user goes to sleep over time, decision support engine 114 may determine the patient is developing kidney disease and/or is at risk of developing kidney disease.

In certain embodiments, a lower autocorrelation score (e.g., less than 0.5) over time may demonstrate worsening kidney disease. In certain embodiments, higher maximum glucose levels and lower minimum glucose levels may demonstrate worsening kidney disease. In certain embodiments, higher glucose rate of change over time may demonstrate that the patient is developing kidney disease and/or is experiencing worsening kidney disease. In certain embodiments, higher mean glucose levels over time and/or higher standard deviation of glucose levels over time may demonstrate that the patient is developing kidney disease and/or is experiencing worsening kidney disease.

In certain embodiments, decision support engine 114 may use a set point metric to determine a patient's kidney disease stage. For example, as kidney disease worsens, there is increased variability in glucose measurements, which may result in less glucose level time in range, specifically within a range of a set point. As glucose levels within a range of the set point become less frequent, decision support engine 114 may determine a user's kidney disease is progressing.

In some cases, method 400 continues at block 410 by decision support engine 114 generating one or more recommendations for treatment, based, at least in part, on the disease prediction generated at block 408. In particular, decision support engine 114 may provide recommendations for the treatment or prevention of kidney disease, such as lifestyle recommendations, medication recommendations, service intervention recommendations, or other recommendations for managing kidney health. Decision support engine 114 may then output such recommendations for treatment to the user (e.g., through application 106).

In certain embodiments, the one or more recommendations generated by decision support engine 114 include: an alert regarding normal or abnormal analyte levels, analyte thresholds, analyte rates of change, analyte clearance rates, and/or analyte variance; a risk of developing kidney disease (e.g., CKD) in the future (e.g., screening of kidney disease risk); a risk of the current presence of kidney disease (e.g., diagnosis of kidney disease); a prediction regarding a current stage of kidney disease (e.g., staging); a risk of adverse event and/or mortality (e.g., a risk stratification); a risk of adverse health events (e.g., cardiac events, hyperkalemia and/or hypokalemia); a risk of adverse kidney health events (e.g., hyperkalemia and/or hypokalemia); a recommendation to seek additional diagnostic testing; a recommendation for treatment or prevention of kidney disease, including diet, medication, lifestyle, alarm/alert, and service intervention recommendations; and/or a risk of other health conditions, other than kidney disease (e.g., liver disease).

Recommendations for the treatment or prevention of kidney disease may, in some cases, be based on a determined optimal balance of, e.g., potassium (e.g., intracellular and extracellular) and insulin levels for a patient. In particular, in certain embodiments, one or more algorithms may be used to determine an optimal balance of potassium and insulin levels of a patient that is then used to form one or more recommendations for the patient regarding a diet, lifestyle change, treatment, insulin dosage, and/or medication.

In certain embodiments, diet recommendations may include a recommendation for the patient to consume a fixed amount of potassium daily, weekly, etc. For example, a patient may be recommended to eat a fixed amount of potassium per day based on a level of potassium the patient's kidney is currently clearing. In certain embodiments, diet recommendations may include a recommendation for the patient to consume a potassium-containing food at a particular time, such as before dosing with insulin or before exercise. Monitoring potassium consumption may help to ensure that excess levels of potassium are not pushed intracellularly (e.g., due to excess insulin) while also ensuring that the potassium consumed is capable of being cleared by the patient's kidney.

In certain embodiments, diet recommendations may include a recommendation for the patient to increase their potassium consumption. For example, a patient may be recommended to increase potassium consumption where excess insulin and/or one or more diuretics are causing significant drops in measured extracellular potassium for the patient (e.g., which may lead to hypokalemia).

In certain embodiments, lifestyle recommendations (e.g., including exercise recommendations, and/or sweat stimulating environmental exposure such as exposure to a sauna) may include a recommendation for the patient to increase their physical activity daily, weekly, etc. given increased physical activity may be one method for removing excess potassium from the body, e.g., through sweating. For example, for patients with hyperkalemia, one or more models may be used to determine when such patients should engage in physical activity based on potassium levels, kidney function, and/or current insulin production/injection for the patient. A patient may be recommended to engage in a modified physical activity schedule or engage in additional rest breaks based on the determined schedule around when patients should engage in physical activity. In certain embodiments, a lifestyle recommendation may be made to optimize sweating while limiting the physical exertion of the patient, in order to reduce potassium, while preventing potassium from increasing due to exercise exertion by the patient. For example, the recommendation may be to spend a time period in the sauna to stimulate sweat without requiring physical exertion of the patient. In certain embodiments, other analyte data or non-analyte data, such as heart rate or respiratory rate data, may be used in combination with potassium data to provide such exercise recommendations.

In certain embodiments, treatment recommendations may include a recommendation for dialysis for the patient. In certain embodiments, determining an optimal balance of potassium and insulin levels of a patient may help to inform whether dialysis is a recommended treatment for the patient. In particular, dialysis is a treatment for kidney failure that rids the body of unwanted toxins, waste products, and excess fluids by filtering a patient's blood. Dialysis helps to keep the potassium, phosphorus, and sodium levels in a patient's body balanced. Thus, understanding the optimal balance of potassium and insulin in the body may help to inform such treatment where dialysis is recommended.

In certain embodiments, service intervention recommendation may include a recommendation for the patient to seek medical attention. For example, in certain embodiments, the service intervention recommendation may indicate to a patient, or another individual with an interest in the patient's well-being, that the patient needs to immediately go to the emergency room and/or contact their health care provider. In certain other embodiments, the service intervention recommendation may automatically alert the health care provider of the patient as to the condition of the patient for intervention by the physician. In certain other embodiments, the service intervention recommendation may alert medical personnel to send aid to the patient, e.g., trigger ambulance services or paramedic services to provide urgent pre-hospital treatment and stabilization to the patient and/or transport of the patient to definitive care. In certain embodiments, decision support engine 114 may make a service intervention recommendation based on a patient's ability to seek medical help and/or the accessibility of the patient to medical help.

In certain embodiments, an insulin dosage recommendation may include a recommendation of a combination dosage, such as insulin/glucose (e.g., to prevent hypoglycemia). In certain embodiments, one or more algorithms may be used to determine the combination dosage to be recommended to the patient. In particular, insulin can be used to treat hyperkalemia such that increased doses of insulin can be used to reduce extracellular potassium. In a CKD patient who is also diabetic and on insulin, and has increasing potassium levels, an algorithm may be created and used for the administration of insulin to avoid or treat hyperkalemia while also preventing hypoglycemia. For example, the algorithm may calculate the amount of insulin needed to reduce potassium levels by a value, X (e.g., where X is a value greater than zero), and the amount of glucose and timing for glucose consumption to prevent hypoglycemia. Further, the insulin dosage recommendation may be individualized per patient to account for differences in insulin resistance across patients. For example, insulin resistance alters the ability of insulin to push potassium into cells; thus, a patient with a higher insulin resistance may require a different insulin dosage as compared to another patient with a different level of insulin resistance. In certain aspects, the individualized insulin dosage recommendation may be modified over time as insulin resistance increases, or decreases, in the patient.

In certain embodiments, medication recommendations may include a recommendation for the patient to take a new medication where the patient has not been previously taking similar medications. In certain embodiments, medication recommendations may include a recommendation for the patient to stop taking a previously prescribed medication, and in some cases, recommend an alternative medication for consumption by the patient. In certain embodiments, medication recommendations may include a recommendation for the patient to take a lower or higher dosage of a previously prescribed medication. In certain embodiments, medication recommendations may include a recommendation for the titration of a dosage or timing of a dosage of medication previously prescribed to the patient to determine an ideal dosage for the patient (e.g., while monitoring kidney and heart health of the user). In certain embodiments, recommendations regarding medications may be generated to reduce a risk of adverse health events.

In certain embodiments, decision support engine 114 may determine CKD in a patient is progressing and correlate such progression to a drug previously prescribed for the patient. Decision support engine 114 may make this determination based on input medication consumption information for the patient (in combination with other factors). In certain embodiments, determining an optimal balance of potassium and insulin levels of a patient, as well as understanding the interplay between potassium levels, glucose levels, insulin, and one or more types of medicines for the patient, may help to inform which medicine (including dosage and frequency) is best suited for the patient.

In certain embodiments, medication recommendations may include a recommendation for the patient to take a potassium binder as an enema rectally. In certain embodiments, medication recommendations may include a recommendation for the patient to take an oral potassium binder, such as Valtassa.

In certain embodiments, medication recommendations may include a recommendation for the patient to discontinue the use of glucose lowering medications (e.g., sulfonylurea) or to titrate the glucose medication to a lower dose. In certain embodiments, the medication recommendation may be based on a decline in kidney function resulting in lower blood glucose levels. In order to prevent dangerous hypoglycemic events, a medication recommendation may instruct the patient to discontinue and/or titrate a glucose lowering medication in response to declining kidney function and lower blood glucose levels.

In certain embodiments, medication recommendations may include a recommendation for the patient to administer particular type of diuretic and/or dosage. As mentioned previously, medication, such as diuretics, may be prescribed to a patient for the purpose of treating excessive fluid accumulation caused by congestive heart failure (CHF), liver failure, and/or nephritic syndrome. Types of diuretics prescribed may include loop diuretics, thiazide and thiazide-like diuretics, and/or potassium-sparing diuretics. Each of the identified diuretic types may be prescribed to a patient for a different purpose. Thus, in certain embodiments, decision support engine 114 may determine the optimal diuretic for prescription based on the health of the patient and the condition(s) of the patient to be treated. For example, a patient with CHF (without kidney disease) may typically be prescribed a thiazide-like diuretic. In particular, thiazide-like diuretics may help to get rid of the excess fluid caused by CHF; however, such diuretics may, in some cases, dehydrate the patient. Dehydration of a patient with impaired kidneys may further damage the kidneys of the patient. In particular, dehydration may clog the kidneys with muscle proteins (myoglobin). Thus, where the patient is only experiencing CHF, and not kidney disease, prescribing the patient a thiazide-like diuretic may not cause significant harm to the patient's kidneys. However, where the patient is experiencing kidney disease, such diuretics may not be optimal for prescription. Accordingly, another diuretic type may be considered.

In certain embodiments, medication recommendations may include a recommendation for the patient to avoid a medication which may put a patient at risk for high or low potassium levels.

Further, in certain embodiments, decision support engine 114 may determine the optimal diuretic for prescription by also considering possible side effects the diuretic prescribed may have on other organs of the patient. By considering the impact different medications have on other organs, decision support engine 114 may aid a patient in managing conditions of other organs within the patient's body. In certain embodiments, CPM 202 may aid in making this determination, at least with respect to the patient's kidneys. For example, CPM 202 may be used to monitor the effect of medication prescribed to a patient for the purpose of treating CHF to better determine its side effect(s) on kidney functions of the patient. Where potassium levels are observed to be decreasing over a period of time after the patient has been prescribed a diuretic for CHF, one may assume the patient is experiencing dehydration, and further conclude, such dehydration may be adversely impacting the kidneys of the patient. Accordingly, a new diuretic may be considered for prescription.

In certain embodiments, alarm/alert recommendations may include a recommendation for the addition of a new type of alarm/alert, the removal of an existing alarm/alert, an increase or decrease in the frequency of existing alarms/alerts, and/or a change in existing threshold levels for existing alarms/alerts configured for a device used by the patient. As mentioned, in certain embodiments, the type of alarms/alerts customized for each particular display device, the number of alarms/alerts customized for each particular display device, the timing of alarms/alerts customized for each particular display device, and/or the threshold levels configured for each of the alarms (e.g., for triggering) are based on the current health of a patient, the state of a patient's kidney, current treatment recommended to a patient, physiological parameters of a patient when experiencing different symptoms stored in user profile 118 for each patient, and/or, in some cases, the kidney disease prediction generated at block 408.

In certain embodiments, where decision support is based on at least GMI and/or clinical A1C measurements, the generated one or more recommendations may include: a risk of developing kidney disease; a risk of the presence of kidney disease; a risk of adverse health events, such as hypoglycemia and hyperglycemia; and/or a recommendation to seek additional diagnostic testing for kidney disease. Such recommendations may, in certain embodiments, be based on differences, changes, and/or other discrepancies between the GMI and clinical A1C measurements.

In certain embodiments, where decision support is based on at least glucose clearance, glucose-insulin clearance, and/or insulin clearance, the generated one or more recommendations may include: a risk of kidney disease; a risk of adverse health events, such as hypoglycemia and hyperglycemia; a risk of adverse health events based on insulin, such as hypoglycemia and hyperglycemia; and/or a recommendation to seek additional diagnostic testing for kidney disease.

In certain embodiments, where decision support is based on at least potassium data, the generated one or more recommendations may include: a risk of kidney disease; a stage of kidney disease; a risk of mortality due to kidney disease; and/or a risk of adverse health events, such as hypokalemia, hyperkalemia, cardiac events, and the like.

In certain embodiments, where decision support is based on at least potassium data and glucose data, the generated one or more recommendations may include: a risk of kidney disease; a stage of kidney disease; a risk of mortality due to kidney disease; and/or a risk of adverse health events, such as hypokalemia, hyperkalemia, hypoglycemia, hyperglycemia, cardiac events, and the like

In certain embodiments, treatment recommendations may include a recommendation to perform a kidney function challenge to further inform kidney health assessment and kidney disease prediction. A kidney function challenge may comprise administering a significant amount of an analyte, or an analyte precursor (e.g., fructose, which may be metabolized into lactate and glucose), such as those described herein, and monitoring the clearance/metabolism of such analyte. For example, a known or estimated amount of analyte may be administered to the patient, preferably orally, and the analyte levels thereafter monitored to determine a clearance rate of the analyte, and/or a peak analyte level (which would be higher for impaired kidneys as compared to healthy kidneys). Generally, the clearance rate may be determined by calculating a slope between an initial analyte value and a baseline value. In certain embodiments, the baseline value may be determined using a patient's historical data. In certain embodiments, the baseline value may be determined from sensor data. The baseline represents the patient's normal analyte levels during periods where significant fluctuations in the analyte levels are not expected, and each user has a different baseline. The baseline may further be determined in part, based on other measurements associated with other analytes (e.g., lactate to indicate exercise, glucose to indicate consumption of food, etc.) and/or non-analyte data (e.g., HR to indicate exercise, time to indicate circadian rhythm). In certain embodiments, treatment recommendations may also include a recommendation for an amount of analyte to administer for a kidney function challenge. During or after performance of the kidney function challenge, decision support engine 114 may generate an alert regarding a determined analyte clearance rate, a change in analyte clearance rate, a recommendation to seek additional kidney disease testing, a risk of kidney disease, a risk of kidney disease progression or regression, and/or a risk of kidney disease stage. In further embodiments, treatment recommendations may further include a recommendation to repeat a kidney function challenge at different times to determine a change in the kidney's ability to clear the analyte, and/or validate the analyte clearance rate. A drop in analyte clearance rate may indicate a decline in kidney function.

In certain embodiments, decision support engine 114 may use one or more other machine-learning models, trained based on patient-specific data and/or population data, to provide recommendations for the treatment or prevention of kidney disease. The algorithms and/or machine-learning models may take into account one or more inputs 128 and/or metrics 130 (e.g., including analyte levels and/or analyte trends) described with respect to FIG. 3 for a patient to determine optimal recommendations for prevention and/or management of the patient's kidney disease. In certain embodiments, the model may look at different patterns of analyte measurements collected for the patient to guide the patient in the management of their disease. Again, the models, and thus, the one or more recommendations generated by decision support engine 114, may be based on analyte levels, analyte thresholds, analyte rates of change, analyte variances, analyte clearance rates, and/or other analyte data.

After generating the one or more recommendations, at block 412, method 400 continues by transmitting an indication (e.g., an alert, alarm, or other type of notification) to a user regarding the kidney disease-related prediction(s) (e.g., predictions as to the presence of abnormal kidney function, risk of kidney disease; and/or presence and/or stage of kidney disease) and/or the generated recommendations (e.g., alarms and/or alerts regarding the risk, presence, and/or severity of kidney disease, recommendations regarding treatment, etc.). In certain embodiments, the indication is transmitted to the patient via application 106, wherein the indication is displayed to the user on display device 107 such as a smart phone or other computing device. In certain embodiments, the indication is transmitted to a health care provider, in addition or alternatively to the patient.

In certain embodiments, any one or more components or devices of decision support system 100 may comprise a “share/follow” function to alarm, alert, provide recommendations to, and share historical and/or projected data with healthcare professionals, clinicians, and/or other caregivers of a patient. For example, such “share/follow” function be comprised on one or more continuous analyte sensors 202 of continuous analyte monitoring system 104, and/or application 106 as executed on display device 107. In certain embodiments, such decision support alarms, alerts, and/or recommendations may be tailored to the healthcare professionals, clinicians, and/or caregivers of the patient, rather than the patient. In certain embodiments, such support alarms, alerts, and/or recommendations may be automatically provided to the healthcare professionals, clinicians, and/or other caregivers of the patient. In certain embodiments, a patient may request decision support system 100 to provide such support alarms, alerts, and/or recommendations to the healthcare professionals, clinicians, and/or other caregivers of the patient, via patient interaction with, e.g., an interface of a display device 107 associated with the patient or a continuous analyte sensors 202. The support alarms, alerts, and/or recommendations may generally be provided to the healthcare professionals, clinicians, and/or other caregivers of the patient through wired/wireless communication, and/or other means of communicating data.

In certain embodiments, machine learning models deployed by decision support engine 114 include one or more models trained by training server system 140, as illustrated in FIG. 1. FIG. 5 describes in further detail techniques for training the machine learning model(s) deployed by decision support engine 114 for generating predictions associated with kidney disease, according to certain embodiments of the present disclosure.

In certain embodiments, method 500 is used to train models to generate, as output, predictions associated with kidney disease. Predications associated with kidney disease may include (1) predictions as to the presence of abnormal kidney function; (2) predictions as to a risk of kidney disease; and (3) predictions as to a presence and/or stage of kidney disease. In certain embodiments, predictions associated with kidney disease may further include predictions as to a risk of adverse health events in a patient (e.g., a user illustrated in FIG. 1), and/or predictions as to an optimal treatment for the patient. In certain embodiments, output generated by models includes a determination of the variation between modeled analyte data and expected analyte data. In certain embodiments, output generated by models may correct and/or corroborate measured analyte data.

Method 500 begins, at block 502, by a training server system, such as training server system 140 illustrated in FIG. 1, retrieving data from a historical records database, such as historical records database 112 illustrated in FIG. 1. As mentioned herein, historical records database 112 may provide a repository of up-to-date information and historical information for users of a continuous analyte monitoring system and connected mobile health application, such as users of continuous analyte monitoring system 104 and application 106 illustrated in FIG. 1, as well as data for one or more patients who are not, or were not previously, users of continuous analyte monitoring system 104 and/or application 106. In certain embodiments, historical records database 112 may include one or more data sets of historical patients with no kidney disease or varying stages of kidney disease (e.g., CKD).

Retrieval of data from historical records database 112 by training server system 140, at block 502, may include the retrieval of all, or any subset of, information maintained by historical records database 112. For example, where historical records database 112 stores information for 100,000 patients (e.g., non-users and users of continuous analyte monitoring system 104 and application 106), data retrieved by training server system 140 to train one or more machine learning models may include information for all 100,000 patients or only a subset of the data for those patients, e.g., data associated with only 50,000 patients or only data from the last ten years.

As an illustrative example, integrating with on premises or cloud based medical record databases through Fast Healthcare Interoperability Resources (FHIR), web application programming interfaces (APIs), Health Level 7 (HL7), and or other computer interface language may enable aggregation of healthcare historical records for baseline assessment in addition to the aggregation of de-identifiable patient data from a cloud based repository.

As an illustrative example, at block 502, training server system 140 may retrieve information for 100,000 patients with varying stages of kidney disease stored in historical records database 112 to train a model to predict the risk, presence, and/or severity of kidney disease in a user. Each of the 100,000 patients may have a corresponding data record (e.g., based on their corresponding user profile)), stored in historical records database 112. Each user profile 118 may include information, such as information discussed with respect to FIG. 3.

The training server system 140 then uses information in each of the records to train an artificial intelligence or ML model (for simplicity referred to as “ML model” herein). Examples of types of information included in a patient's user profile were provided above. The information in each of these records may be featurized (e.g., manually or by training server system 140), resulting in features that can be used as input features for training the ML model. For example, a patient record may include or be used to generate features related to an age of a patient, a gender of the patient, an occupation of the patient, analyte levels for the patient over time, analyte level rates of change and/or trends for the patient over time, physiological parameters associated with different kidney disease stages for the patient over time, and/or any information provided by inputs 128 and/or metrics 130, etc. Features used to train the machine learning model(s) may vary in different embodiments.

In certain embodiments, each historical patient record retrieved from historical records database 112 is further associated with a label indicating whether the patient was healthy or experienced some variation of kidney disease, a previously determined kidney disease diagnosis and/or stage of chronic kidney disease (CKD) for the patient, a kidney disease risk assessment, treatment(s), and/or similar metrics. What the record is labeled with would depend on what the model is being trained to predict.

At block 504, method 500 continues by training server system 140 training one or more machine learning models based on the features and labels associated with the historical patient records. In some embodiments, the training server does so by providing the features as input into a model. This model may be a new model initialized with random weights and parameters, or may be partially or fully pre-trained (e.g., based on prior training rounds). Based on the input features, the model-in-training generates some output. In certain embodiments, the output may indicate a kidney disease diagnosis and/or stage for the patient, a risk assessment associated with the patient developing kidney disease, as well as an assessment around improvement in or deterioration of the patient's existing kidney disease. In certain embodiments, the output may indicate a level of risk the patient may develop kidney disease in the future.

In certain embodiments, training server system 140 compares this generated output with the actual label associated with the corresponding historical patient record to compute a loss based on the difference between the actual result and the generated result. This loss is then used to refine one or more internal weights and parameters of the model (e.g., via backpropagation) such that the model learns to predict the presence and/or severity of kidney disease (or its recommended treatments) more accurately.

One of a variety of machine learning algorithms may be used for training the model(s) described above. For example, one of a supervised learning algorithm, a neural network algorithm, a deep neural network algorithm, a deep learning algorithm, etc. may be used.

At block 506, training server system 140 deploys the trained model(s) to make predictions associated with kidney disease during runtime. In some embodiments, this includes transmitting some indication of the trained model(s) (e.g., a weights vector) that can be used to instantiate the model(s) on another device. For example, training server system 140 may transmit the weights of the trained model(s) to decision support engine 114. The model(s) can then be used to assess, in real-time, the presence and/or severity of kidney disease of a user using application 106, provide treatment recommendations, and/or make other types of predictions discussed above. In certain embodiments, the training server system 140 may continue to train the model(s) in an “online” manner by using input features and labels associated with new patient records.

Further, similar methods for training illustrated in FIG. 5 using historical patient records may also be used to train models using patient-specific records to create more personalized models for making predictions associated with kidney disease. For example, a model trained using historical patient records that is deployed for a particular user, may be further re-trained after deployment. For example, the model may be re-trained after the model is deployed for a specific patient to create a more personalized model for the patient. The more personalized model may be able to more accurately make kidney disease-related predictions for the patient based on the patient's own data (as opposed to only historical patient record data), including the patient's own analyte (e.g., potassium) thresholds.

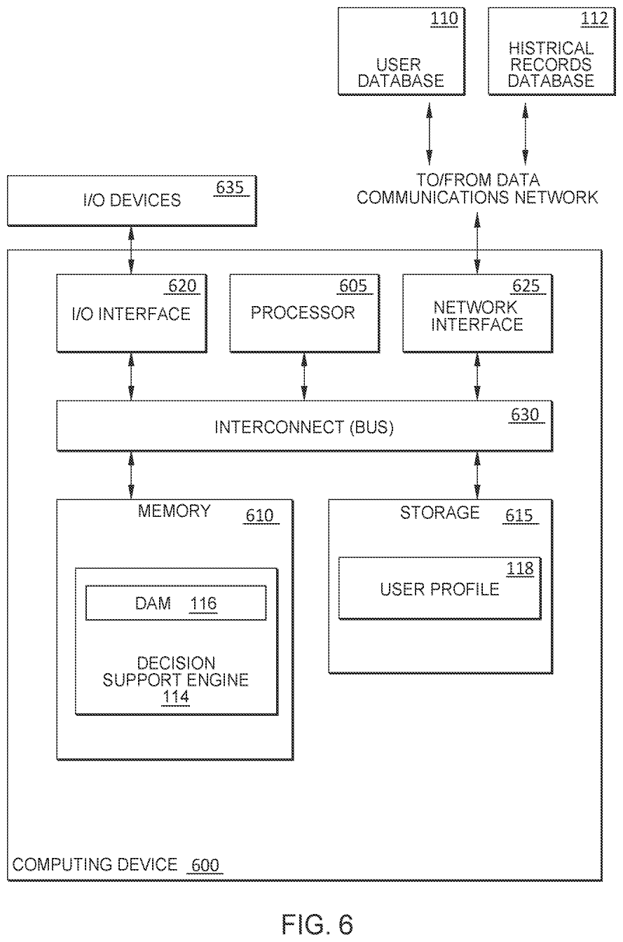

FIG. 6 is a block diagram depicting a computing device 600 configured for diagnosing, staging, treating, and assessing risks of kidney disease, according to certain embodiments disclosed herein. Although depicted as a single physical device, in embodiments, computing device 600 may be implemented using virtual device(s), and/or across a number of devices, such as in a cloud environment. As illustrated, computing device 600 includes a processor 605, memory 610, storage 615, a network interface 625, and one or more I/O interfaces 620. In the illustrated embodiment, processor 605 retrieves and executes programming instructions stored in memory 610, as well as stores and retrieves application data residing in storage 615. Processor 605 is generally representative of a single CPU and/or GPU, multiple CPUs and/or GPUs, a single CPU and/or GPU having multiple processing cores, and the like. Memory 610 is generally included to be representative of a random access memory (RAM). Storage 615 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 635 (such as keyboards, monitors, etc.) can be connected via the I/O interface(s) 620. Further, via network interface 625, computing device 600 can be communicatively coupled with one or more other devices and components, such as user database 110 and/or historical records database 112. In certain embodiments, computing device 600 is communicatively coupled with other devices via a network, which may include the Internet, local network(s), and the like. The network may include wired connections, wireless connections, or a combination of wired and wireless connections. As illustrated, processor 605, memory 610, storage 615, network interface(s) 625, and I/O interface(s) 620 are communicatively coupled by one or more interconnects 630. In certain embodiments, computing device 600 is representative of display device 107 associated with the user. In certain embodiments, as discussed above, display device 107 can include the user's laptop, computer, smartphone, and the like. In another embodiment, computing device 600 is a server executing in a cloud environment.

In the illustrated embodiment, storage 615 includes user profile 118. Memory 610 includes decision support engine 16, which itself includes DAM 116. Decision support engine 114 is executed by computing device 600 to perform operations in method 400 of FIG. 4 and operations of method 500 in FIG. 5 for providing decision support in the form of risk assessment and treatment for kidney disease (e.g., CKD).

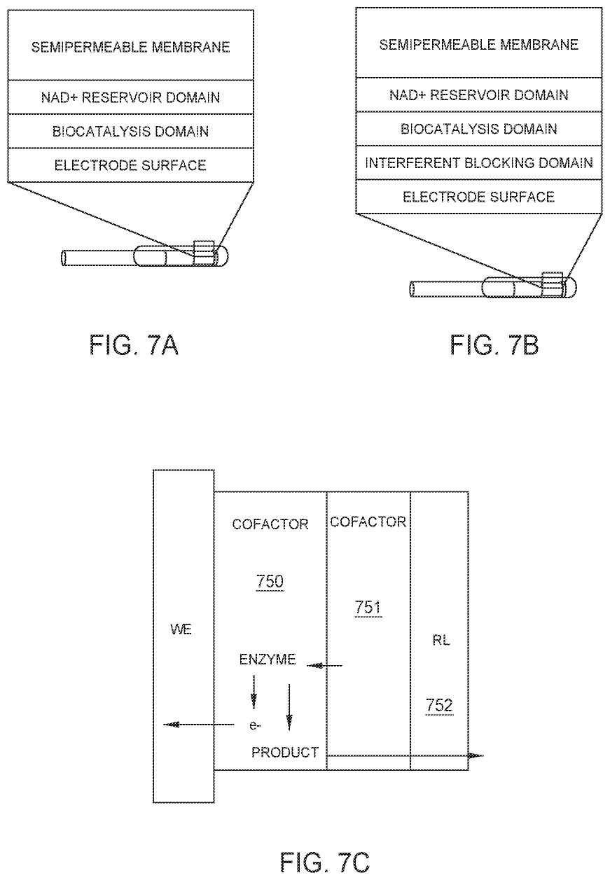

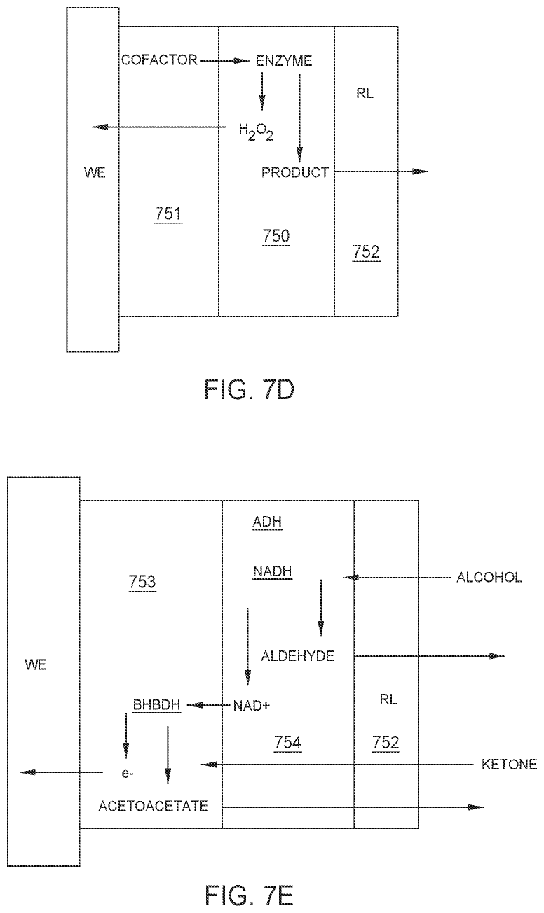

As described above, continuous analyte monitoring system 104, described in relation to FIG. 1, may be a multi-analyte sensor system including a multi-analyte sensor. FIGS. 7-11 describe example multi-analyte sensors used to measure multiple analytes.

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms can provide specific quantitative, semi-quantitative, qualitative, semi qualitative analytical signals using a biological recognition element combined with a transducing (detecting) element.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. Analyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7, 10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components, covalently, electrostatically, ionically associated with, or physically entrapped, or absorbed

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In one example this reaction is faradaic and results in charge transfer between the surface and its environment. In one example, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds which produce a signal that is not analyte-specific due to a reaction on an electrochemically active surface.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In one example the mediator accepts electrons from, or transfer electrons to, one or more enzymes or cofactors, and/or exchanges electrons with the sensor system electrodes. In one example, mediators are transition-metal coordinated organic molecules which are capable of reversible oxidation and reduction reactions. In other examples, mediators may be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In one example, the membrane system comprises an enzyme, which enables an analyte reaction to occur whereby a concentration of the analyte can be measured.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In one example, planar includes one or more edges separating the opposed surfaces.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms are capable of providing specific quantitative, semi-quantitative, qualitative, semi qualitative analytical signals using a biological recognition element combined with a transducing and/or detecting element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, ketone, lactate, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include current and/or voltage, inductance, capacitance, impedance, transconductance, and potential. Optical properties include absorbance, fluorescence/phosphorescence, fluorescence/phosphorescence decay rate, wavelength shift, dual wave phase modulation, bio/chemiluminescence, reflectance, light scattering, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more DNA or RNA moieties. Transcutaneous continuous multi-analyte sensors can be used in vivo over various lengths of time. The continuous multi-analyte sensor systems discussed herein can be transcutaneous devices, in that a portion of the device may be inserted through the host's skin and into the underlying soft tissue while a portion of the device remains on the surface of the host's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the host's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, spraying, electrodepositing, dipping, brush coating, film coating, drop-let coating, and the like). Additional steps may be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (m), or less, to about 1, 2, 3, 4, 5, 6, 7, 8, 9, 10, 11, 12, 13, 14, 15, or 16 m is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl)carbodiimide (EDC)) and cured at a moderate temperature of about 50° C.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro)chemical reactions involved in sensing, but forms chemical bonds with the (electro)chemically active components of the membrane. In some examples these bonds are covalent. In one example, a tether may be formed in solution prior to one or more interlayers of a membrane being formed, where the tether bonds two (electro)chemically active components directly to one another or alternately, the tether(s) bond (electro)chemically active component(s) to polymeric backbone structures. In another example, (electro)chemically active components are comixed along with crosslinker(s) with tunable lengths (and optionally polymers) and the tethering reaction occurs as in situ crosslinking. Tethering may be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as spraying, dipping, casting, electrospinning, vapor deposition, spin coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEGDE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spraying, self-assembling monolayers (SAMs), painting, dip coating, vapor depositing, molding, 3-D printing, lithographic techniques (e.g., photolithograph), micro- and nano-pipetting printing techniques, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor may promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein may include a biointerface layer. The biointerface layer, like the drug releasing layer, may include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein may include a drug releasing membrane at least partially functioning as or in combination with a biointerface membrane. The drug releasing membrane may include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmetharcrylate and copolymers and blends thereof, and hydroxyapeptite and copolymers and blends thereof.

### Exemplary Multi-Analyte Sensor Membrane Configurations

Continuous multi-analyte sensors with various membrane configurations suitable for facilitating signal transduction corresponding to analyte concentrations, either simultaneously, intermittently, and/or sequentially are provided. In one example, such sensors can be configured using a signal transducer, comprising one or more transducing elements (“TL”). Such continuous multi-analyte sensor can employ various transducing means, for example, amperometry, voltametric, potentiometry, and impedimetric methods, among other techniques.

In one example, the transducing element comprises one or more membranes that can comprise one or more layers and or domains, each of the one or more layers or domains can independently comprise one or more signal transducers, e.g., enzymes, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain may comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte-substrate/enzyme pairs: for example, sarcosine oxidase in combination with creatinine amidohydrolase, creatine amidohydrolase being employed for the sensing of creatinine. Other examples of analytes/oxidase enzyme combinations that can be used in the sensing region include, for example, alcohol/alcohol oxidase, cholesterol/cholesterol oxidase, glactose:galactose/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine:xanthine/xanthine oxidase, glucose/glucose oxidase, lactate/lactate oxidase, L-amino acid oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, dimerized and/or fusion enzymes.

### NAD Based Multi-Analyte Sensor Platform

Nicotinamide adenine dinucleotide (NAD(P)+/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)+) and reduced form (NAD(P)H) (H=hydrogen). The reaction of NAD+ and NADH is reversible, thus, the coenzyme can continuously cycle between the NAD(P)⁺/and NAD(P)H forms essentially without being consumed.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, NAD, NADH, NAD+, NAD(P)+, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD(P)+, ATP, flavin adenine dinucleotide (FAD), magnesium (Mg++), pyrroloquinoline quinone (PQQ), and functionalized derivatives are incorporated in the sensing region. In one example, NAD, NADH, NAD+, NAD(P)+, ATP, flavin adenine dinucleotide (FAD), magnesium (Mg++), pyrroloquinoline quinone (PQQ), and functionalized derivatives are dispersed or distributed in one or more membranes or domains of the sensing region.

In one aspect of the present disclosure, continuous sensing of one or more or two or more analytes using NAD+ dependent enzymes is provided in one or more membranes or domains of the sensing region. In one example, the membrane or domain provides retention and stable recycling of NAD+ as well as mechanisms for transducing NADH oxidation or NAD+ reduction into measurable current with amperometry. In one example, described below, continuous, sensing of multi-analytes, either reversibly bound or at least one of which are oxidized or reduced by NAD+ dependent enzymes, for example, ketones (beta-hydroxybutyrate dehydrogenase), glycerol (glycerol dehydrogenase), cortisol (11β-hydroxysteroid dehydrogenase), glucose (glucose dehydrogenase), alcohol (alcohol dehydrogenase), aldehydes (aldehyde dehydrogenase), and lactate (lactate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 7A. With reference to FIG. 7B, one or more optional layers may be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 7A-7B, one or more of the membranes provides a NAD+ reservoir domain providing a reservoir for NAD+. In one example, one or more interferent blocking membranes is used, and potentiostat is utilized to measure H2O2 production or O2 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein.

In one example, one or more mediators that are optimal for NADH oxidation are incorporated in the one or more electrode domains or enzyme domains. In one example, organic mediators, such as phenanthroline dione, or nitrosoanilines are used. In another example, metallo-organic mediators, such as ruthenium-phenanthroline-dione or osmium(bpy)₂Cl, polymers containing covalently coupled organic mediators or organometallic coordinated mediators polymers for example polyvinylimidizole-Os(bpy)₂Cl, or polyvinylpyridine-organometallic coordinated mediators (including ruthenium-phenanthroline dione) are used. Other mediators can be used as discussed further below.

In humans, serum levels of beta-hydroxybutyrate (BHB) are usually in the low micromolar range but can rise up to about 6-8 mM. Serum levels of BHB can reach 1-2 mM after intense exercise or consistent levels above 2 mM are reached with a ketogenic diet that is almost devoid of carbohydrates. Other ketones are present in serum, such as acetoacetate and acetone, however, most of the dynamic range in ketone levels is in the form of BHB. Thus, monitoring of BHB, e.g., continuous monitoring is useful for providing health information to a user or health care provider.

Another example of a continuous ketone analyte detection configuration employing electrode-associated mediator-coupled diaphorase/NAD+/dehydrogenase is depicted below:

$$\begin{matrix}{Mediator}_{ox} \\ {Mediator}_{red}\end{matrix} \begin{matrix}{diaphorase}_{ox} \\ {diaphorase}_{red}\end{matrix} \begin{matrix}NAD + \\ NADH\end{matrix}$$

$$\begin{matrix}{Dehydrogenase}_{ox} ( e . g . {HBDH}_{ox} ) \\ {Dehydrogenase}_{red} ( e . g . {HBDH}_{red} )\end{matrix} \begin{matrix}Oxidized analyte ( e . g . Acetoacetate ) \\ Analyte ( e . g . hydroxybutyrate )\end{matrix}$$

In one example, the diaphorase is electrically coupled to the electrode with organometallic coordinated mediator polymer. In another example, the diaphorase is covalently coupled to the electrode with an organometallic coordinated mediator polymer. Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte (ketone) oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ may be covalently coupled to an aspect of the enzyme domain having a higher molecular weight than the NAD+ which may improve a stability profile of the NAD+, improving the ability to retain and/or immobilize the NAD+ in the enzyme domain. For example, dextran-NAD.

In one example, the sensing region comprises one or more NADH:acceptor oxidoreductases and one or more NAD-dependent dehydrogenases. In one example, sensing region comprises one or more NADH:acceptor oxidoreductases and one or more NAD(P)-dependent dehydrogenases with NAD(P)+ or NAD(P)H as cofactors present in sensing region. In one example, the sensing region comprises an amount of diaphorase.

In one example, a ketone sensing configuration suitable for combination with another analyte sensing configuration is provided. Thus, an EZL layer of about 1-20 um thick is prepared by presenting a EZL solution composition in 10 mM HEPES in water having about 20 uL 500 mg/mL HBDH, about 20 uL [500 mg/mL NAD(P)H, 200 mg/mL polyethylene glycol-diglycol ether (PEG-DGE) of about 400 MW], about 20 uL 500 mg/mL diaphorase, about 40 uL 250 mg/mL poly vinyl imidazole-osmium bis(2,2′-bipyridine)chloride (PVI-Os(bpy)2Cl) to a substrate such as a working electrode, so as to provide, after drying, about 15-40% by weight HBDH, about 5-30% diaphorase about 5-30% NAD(P)H, about 10-50% PVI-Os(bpy)2Cl and about 1-12% PEG-DGE(400 MW). The substrates discussed herein that may include working electrodes may be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well combinations thereof.

To the above enzyme domain was contacted a resistance domain, also referred to as a resistance layer (“RL”). In one example, the RL comprises about 55-100% PVP, and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous ketone sensor as depicted in FIGS. 7A-7B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) may also contain a polymer or protein binder, such as zwitterionic polyurethane, and/or albumin. Alternatively, in addition to NAD(P)H, the membrane may contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer (or protein), and may be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NADH oxidase. In one example, NADH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE.

The aforementioned continuous ketone sensor configurations can be adapted to other analytes or used in combination with other sensor configurations. For example, analyte(s)-dehydrogenase enzyme combinations can be used in any of the membranes of the sensing region include; glycerol (glycerol dehydrogenase); cortisol (11β-hydroxysteroid dehydrogenase); glucose (glucose dehydrogenase); alcohol (alcohol dehydrogenase); aldehydes (aldehyde dehydrogenase); and lactate (lactate dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semipermeable membrane can be an ion selective membrane selective for an ion analyte of interest, such as ammonium ion.

In another example, a continuous multi-analyte sensor configuration comprising one or more enzymes and/or at least one cofactor was prepared. FIG. 7C depicts this exemplary configuration, of an enzyme domain 750 comprising an enzyme (Enzyme) with an amount of cofactor (Cofactor) that is positioned proximal to at least a portion of a working electrode (“WE”) surface, where the WE comprises an electrochemically reactive surface. In one example, a second membrane 751 comprising an amount of cofactor is positioned adjacent the first enzyme domain. The amount of cofactor in the second membrane can provide an excess for the enzyme, e.g., to extend sensor life. One or more resistance domains 752 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIG. 7D depicts an alternative enzyme domain configuration comprising a first membrane 751 with an amount of cofactor that is positioned more proximal to at least a portion of a WE surface. Enzyme domain 750 comprising an amount of enzyme is positioned adjacent the first membrane.

In the membrane configurations depicted in FIGS. 7C-7D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as Mg⁺². One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

FIG. 7E depicts another continuous multi-analyte membrane configuration, where {beta}-hydroxybutyrate dehydrogenase BHBDH in a first enzyme domain 753 is positioned proximate to a working electrode WE and second enzyme domain 754, for example, comprising alcohol dehydrogenase (ADH) and NADH is positioned adjacent the first enzyme domain. One or more resistance domains RL 752 may be deployed adjacent to the second enzyme domain 754. In this configuration, the presence of the combination of alcohol and ketone in serum works collectively to provide a transduced signal corresponding to at least one of the analyte concentrations, for example, ketone. Thus, as the NADH present in the more distal second enzyme domain consumes alcohol present in the serum environment, NADH is oxidized to NAD(P)H that diffuses into the first membrane layer to provide electron transfer of the BHBDH catalysis of acetoacetate ketone and transduction of a detectable signal corresponding to the concentration of the ketone. In one example, an enzyme can be configured for reverse catalysis and can create a substrate used for catalysis of another enzyme present, either in the same or different layer or domain. Other configurations can be used in the aforementioned configuration, such as electrode, resistance, bio-interfacing, and drug releasing membranes, layers, or domains. Thus, a first enzyme domain that is more distal from the WE than a second enzyme domain may be configured to generate a cofactor or other element to act as a reactant (and/or a reactant substrate) for the second enzyme domain to detect the one or more target analytes.

### Alcohol Sensor Configurations

In one example, a continuous alcohol (e.g., ethanol) sensor device configuration is provided. In one example, one or more enzyme domains comprising alcohol oxidase (AOX) is provided and the presence and/or amount of alcohol is transduced by creation of hydrogen peroxide, alone or in combination with oxygen consumption or with another substrate-oxidase enzyme system, e.g., glucose-glucose oxidase, in which hydrogen peroxide and or oxygen and/or glucose can be detected and/or measured qualitatively or quantitatively, using amperometry.

In one example, the sensing region for the aforementioned enzyme substrate-oxidase enzyme configurations has one or more enzyme domains comprises one or more electrodes. In one example, the sensing region for the aforementioned enzyme substrate-oxidase enzyme configurations has one or more enzyme domains, with or without the one or more electrodes, further comprises one or interference blocking membranes (e.g. permselective membranes, charge exclusion membranes) to attenuate one or more interferents from diffusing through the membrane to the working electrode. In one example, the sensing region for the aforementioned substrate-oxidase enzyme configurations has one or more enzyme domains, with or without the one or more electrodes, and further comprises one or resistance domains with or without the one or more interference blocking membranes to attenuate one or more analytes or enzyme substrates. In one example, the sensing region for the aforementioned substrate-oxidase enzyme configurations has one or more enzyme domains, with or without the one or more electrodes, one or more resistance domains with or without the one or more interference blocking membranes further comprises one or biointerface membranes and/or drug releasing membranes, independently, to attenuate one or more analytes or enzyme substrates and attenuate the immune response of the host after insertion.

In one example, the one or more interference blocking membranes are deposited adjacent the working electrode and/or the electrode surface. In one example, the one or interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing thru the sensing region but for oxygen. Such membranes can be used to attenuate alcohol itself as well as attenuate other electrochemically actives species or other analytes that can otherwise interfere by producing a signal if they diffuse to the working electrode.

In one example, the working electrode used comprised platinum and the potential applied was about 0.5 volts.

In one example, sensing oxygen level changes electrochemically, for example in a Clark type electrode setup, or in a different configuration can be carried out, for example by coating the electrode with one or more membranes of one or more polymers, such as NAFION™. Based on changes of potential, oxygen concentration changes can be recorded, which correlate directly or indirectly with the concentrations of alcohol. When appropriately designed to obey stoichiometric behavior, the presence of a specific concentration of alcohol should cause a commensurate reduction in local oxygen in a direct (linear) relation with the concentration of alcohol. Accordingly, a multi-analyte sensor for both alcohol and oxygen can therefore be provided.

In another example, the above mentioned alcohol sensing configuration can include one or more secondary enzymes that react with a reaction product of the alcohol/alcohol oxidase catalysis, e.g., hydrogen peroxide, and provide for a oxidized form of the secondary enzyme that transduces an alcohol-dependent signal to the WE/RE at a lower potential than without the secondary enzyme. Thus, in one example, the alcohol/alcohol oxidase is used with a reduced form of a peroxidase, for example horse radish peroxidase. The alcohol/alcohol oxidase can be in same or different layer as the peroxidase, or they may be spatially separated distally from the electrode surface, for example, the alcohol/alcohol oxidase being more distal from the electrode surface and the peroxidase being more proximal to the electrode surface, or alternatively, the alcohol/alcohol oxidase being more proximal from the electrode surface and the peroxidase being more distal to the electrode surface. In one example, the alcohol/alcohol oxidase, being more distal from the electrode surface and the peroxidase, further includes any combination of electrode, interference, resistance, and biointerface membranes to optimize signal, durability, reduce drift, or extend end of use duration.

In another example, the above mentioned alcohol sensing configuration can include one or more mediators. In one example, the one or more mediators are present in, on, or about one or more electrodes or electrode surfaces and/or are deposited or otherwise associated with the surface of the working electrode (WE) or reference electrode (RE). In one example, the one or more mediators eliminate or reduce direct oxidation of interfering species that may reach the WE or RE. In one example, the one or more mediators provide a lowering of the operating potential of the WE/RE, for example, from about 0.6V to about 0.3V or less on a platinum electrode, which can reduce or eliminates oxidation of endogenous interfering species. Examples of one or mediators are provided below. Other electrodes, e.g., counter electrodes, can be employed.

In one example, other enzymes or additional components may be added to the polymer mixture(s) that constitute any part of the sensing region to increase the stability of the aforementioned sensor and/or reduce or eliminate the biproducts of the alcohol/alcohol oxidase reaction. Increasing stability includes storage or shelf life and/or operational stability (e.g., retention of enzyme activity during use). For example, byproducts of enzyme reactions may be undesirable for increased shelf life and/or operational stability, and may thus be desirable to reduce or remove. In one example, xanthine oxidase can be used to remove bi-products of one or more enzyme reactions.

In another example, a dehydrogenase enzyme is used with an oxidase for the detection of alcohol alone or in combination with oxygen. Thus, in one example, alcohol dehydrogenase is used to oxidize alcohol to aldehyde in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor.

In the aforementioned dual enzyme configuration, a signal can be sensed either by: (1) an electrically coupled (e.g., “wired”) alcohol dehydrogenase (ADH), for example, using an electro-active hydrogel polymer comprising one or more mediators; or (2) oxygen electrochemical sensing to measure the oxygen consumption of the NADH oxidase. In an alternative example, the co-factor NAD(P)H or NAD(P)+ may be coupled to a polymer, such as dextran, the polymer immobilized in the enzyme domain along with ADH. This provides for retention of the co-factor and availability thereof for the active site of ADH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface is provided. A chemical moiety capable of assisting with electron transfer from the enzyme or cofactor to the electrode surface can be used and includes one or more mediators as described below.

In one example, any one of the aforementioned continuous alcohol sensor configurations are combined with any one of the aforementioned continuous ketone monitoring configurations to provide a continuous multi-analyte sensor device as further described below. In one example a continuous glucose monitoring configuration combined with any one of the aforementioned continuous alcohol sensor configurations and any one of the aforementioned continuous ketone monitoring configurations to provide a continuous multi-analyte sensor device as further described below.

### Uric Acid Sensor Configurations

In another example, a continuous uric acid sensor device configuration is provided. Thus, in one example, uric acid oxidase (UOX) can be included in one or more enzyme domains and positioned adjacent the working electrode surface. The catalysis of the uric acid using UOX, produces hydrogen peroxide which can be detected using, among other techniques, amperometry, voltametric and impedimetric methods. In one example, to reduce or eliminate the interference from direct oxidation of uric acid on the electrode surface, one or more electrode, interference, and/or resistance domains can be deposited on at least a portion of the working electrode surface. Such membranes can be used to attenuate diffusion of uric acid as well as other analytes to the working electrode that can interfere with signal transduction.

In one alternative example, a uric acid continuous sensing device configuration comprises sensing oxygen level changes about the WE surface, e.g., for example, as in a Clark type electrode setup, or the one or more electrodes can comprise, independently, one or more different polymers such as NAFION™, polyzwitterion polymers, or polymeric mediator adjacent at least a portion of the electrode surface. In one example, the electrode surface with the one or more electrode domains provide for operation at a different or lower voltage to measure oxygen. Oxygen level and its changes in can be sensed, recorded, and correlated to the concentration of uric acid based using, for example, using conventional calibration methods.

In one example, alone or in combination with any of the aforementioned configurations, uric acid sensor configurations, so as to lower the potential at the WE for signal transduction of uric acid, one or more coatings can be deposited on the WE surface. The one or more coatings may be deposited or otherwise formed on the WE surface and/or on other coatings formed thereon using various techniques including, but not limited to, dipping, electrodepositing, vapor deposition, spray coating, etc. In one example, the coated WE surface can provide for redox reactions, e.g., of hydrogen peroxide, at lower potentials (as compared to 0.6 V on platinum electrode surface without such a coating. Example of materials that can be coated or annealed onto the WE surface includes, but are not limited to Prussian Blue, Medola Blue, methylene blue, methylene green, methyl viologen, ferrocyanide, ferrocene, cobalt ion, and cobalt phthalocyanine, and the like.

In one example, one or more secondary enzymes, cofactors and/or mediators (electrically coupled or polymeric mediators) can be added to the enzyme domain with UOX to facilitate direct or indirect electron transfer to the WE. In such configurations, for example, regeneration of the initial oxidized form of secondary enzyme is reduced by the WE for signal transduction. In one example, the secondary enzyme is horse radish peroxidase (HRP).

### Choline Sensor Configurations

In one example continuous choline sensor device can be provided, for example, using choline oxidase enzyme that generates hydrogen peroxide with the oxidation of choline. Thus, in one example, at least one enzyme domain comprises choline oxidase (COX) adjacent at least one WE surface, optionally with one or more electrodes and/or interference membranes positioned in between the WE surface and the at least one enzyme domain. The catalysis of the choline using COX results in creation of hydrogen peroxide which can be detectable using, among other techniques, amperometry, voltametric and impedimetric methods.

In one example, the aforementioned continuous choline sensor configuration is combined with any one of the aforementioned continuous alcohol sensor configurations, and continuous uric acid sensor configurations to provide a continuous multi-analyte sensor device as further described below. This continuous multi-analyte sensor device can further include continuous glucose monitoring capability. Other membranes can be used in the aforementioned continuous choline sensor configuration, such as electrode, resistance, bio-interfacing, and drug releasing membranes.

### Cholesterol Sensor Configurations

In one example, continuous cholesterol sensor configurations can be made using cholesterol oxidase (CHOX), in a manner similar to previously described sensors. Thus, one or more enzyme domains comprising CHOX can be positioned adjacent at least one WE surface. The catalysis of free cholesterol using CHOX results in creation of hydrogen peroxide which can be detectable using, among other techniques, amperometry, voltametric and impedimetric methods.

An exemplary cholesterol sensor configuration using a platinum WE, where at least one interference membrane is positioned adjacent at least one WE surface, over which there is at least one enzyme domain comprising CHOX, over which is positioned at least one resistance domain to control diffusional characteristics was prepared.

The method described above and the cholesterol sensors described can measure free cholesterol, however, with modification, the configuration can measure more types of cholesterol as well as total cholesterol concentration. Measuring different types of cholesterol and total cholesterol is important, since due to low solubility of cholesterol in water significant amount of cholesterol is in unmodified and esterified forms. Thus, in one example, a total cholesterol sample is provided where a secondary enzyme is introduced into the at least one enzyme domain, for example, to provide the combination of cholesterol esterase with CHOX Cholesteryl ester, which essentially represents total cholesterols can be measured indirectly from signals transduced from cholesterol present and formed by the esterase.

In one example, the aforementioned continuous (total) cholesterol sensor configuration is combined with any one of the aforementioned continuous alcohol sensor configurations and/or continuous uric acid sensor configurations to provide a continuous multi-analyte sensor system as further described below. This continuous multi-analyte sensor device can further include continuous glucose monitoring capability. Other membrane configurations can be used in the aforementioned continuous cholesterol sensor configuration, such as one or more electrode domains, resistance domains, bio-interfacing domains, and drug releasing membranes.

### Bilirubin Sensor and Ascorbic Acid Sensor Configurations

In one example, continuous bilirubin and ascorbic acid sensors are provided. These sensors can employ bilirubin oxidase and ascorbate oxidase, respectively. However, unlike some oxidoreductase enzymes, the final product of the catalysis of analytes of bilirubin oxidase and ascorbate oxidase is water instead of hydrogen peroxide. Therefore, redox detection of hydrogen peroxide to correlate with bilirubin or ascorbic acid is not possible. However, these oxidase enzymes still consume oxygen for the catalysis, and the levels of oxygen consumption correlates with the levels of the target analyte present. Thus, bilirubin and ascorbic acid levels can be measured indirectly by electrochemically sensing oxygen level changes, as in a Clark type electrode setup, for example.

Alternatively, a different configuration for sensing bilirubin and ascorbic acid can be employed. For example, an electrode domain including one or more electrode domains comprising electron transfer agents, such as NAFION™, polyzwitterion polymers, or polymeric mediator can be coated on the electrode. Measured oxygen levels transduced from such enzyme domain configurations can be correlated with the concentrations of bilirubin and ascorbic acid levels. In one example, an electrode domain comprising one or more mediators electrically coupled to a working electrode can be employed and correlated to the levels of bilirubin and ascorbic acid levels.

In one example, the aforementioned continuous bilirubin and ascorbic acid sensor configurations can be combined with any one of the aforementioned continuous alcohol sensor configurations, continuous uric acid sensor configurations, continuous cholesterol sensor configurations to provide a continuous multi-analyte sensor device as further described below. This continuous multi-analyte sensor device can further include continuous glucose monitoring capability. Other membranes can be used in the aforementioned continuous bilirubin and ascorbic acid sensor configuration, such as electrode, resistance, bio-interfacing, and drug releasing membranes.

### One-Working-Electrode Configurations for Dual Analyte Detection

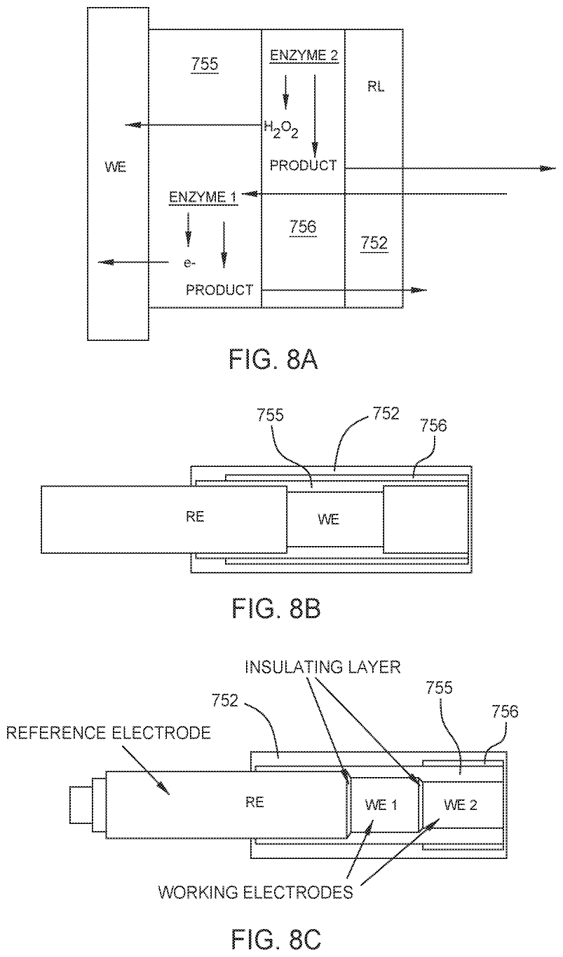

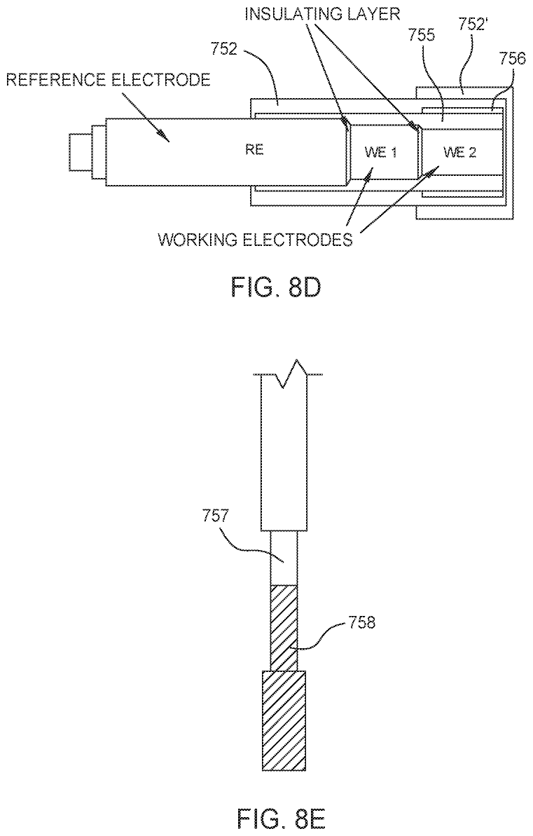

In one example, at least a dual enzyme domain configuration in which each layer contains one or more specific enzymes and optionally one or more cofactors is provided. In a broad sense, one example of a continuous multi-analyte sensor configuration is depicted in FIG. 8A where a first membrane 755 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 756 (EZL2) with at least one second enzyme (Enzyme 2) is positioned adjacent 755 ELZ1, and is generally more distal from WE than EZL1. One or more resistance domains (RL) 752 can be provided adjacent EZL2 756, and/or between EZL1 755 and EZL2 756. The different enzymes catalyze the transformation of the same analyte, but at least one enzyme in EZL2 756 provides hydrogen peroxide and the other at least one enzyme in EZL1 755 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 8A, a first analyte diffuses through RL 752 and into EZL2 756 resulting in peroxide via interaction with Enzyme 2. Peroxide diffuses at least through EZL1 755 to WE and transduces a signal that corresponds directly or indirectly to the first analyte concentration. A second analyte, which is different from the first analyte, diffuses through RL 752 and EZL2 756 and interacts with Enzyme 1, which results in electron transfer to WE and transduces a signal that corresponds directly or indirectly to the second analyte concentration.

As shown in FIG. 8B, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 756 providing hydrogen peroxide and the at least other enzyme in EZL1 755 not providing hydrogen peroxide, e.g., providing electron transfer to the WE surface corresponding directly or indirectly to a concentration of the analyte.

In one example, an inner layer of the at least two enzyme domains EZL1, EZL2 755, 756 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 755 is more proximal to the WE surface and may have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 755 is directly adjacent the WE.

The second layer of at least dual enzyme domain (the outer layer EZL2 756) of FIG. 8B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 756 and through the inner layer EZL1 755 to reach the WE surface and undergoes redox at a potential of P2, where P2≠P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry etc. In some examples, impedimetric sensing may be used. In one example, a phase shift (e.g., a time lag) may result from detecting two signals from two different working electrodes, each signal being generated by a different EZL (EZL1, EZL2, 755, 756) associated with each electrode. The two (or more) signals can be broken down into components to detect the individual signal and signal artifacts generated by each of EZL1 755 and EZL2 756 in response to the detection of two analytes. In some examples, each EZL detects a different analyte. In other examples, both EZLs detect the same analyte.

In another alternative exemplary configuration, as shown in FIGS. 8C-8D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2). In one example, the first analyte detected by WE1 is glucose, and the second analyte detected by WE2 is lactate. In another example, the first analyte detected by WE1 is glucose, and the second analyte detected by WE2 is ketones.

Thus, FIGS. 8C-8D depict exemplary configurations of a continuous multi-analyte sensor construct in which EZL1 755, EZL2 756 and RL 752 (resistance domain) as described above, arranged, for example, by sequential dip coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 8C-8D, WE1 represents a first working electrode surface configured to operate at P1, for example, and is electrically insulated from second working electrode surface WE2 that is configured to operate at P2, and RE represents a reference electrode RE electrically isolated from both WE1, WE2. One resistance domain is provided in the configuration of FIG. 8C that covers the reference electrode and WE1, WE2. An addition resistance domain is provided in the configuration of FIG. 8D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different mode of measurements provides increase fidelity, improved performance and device longevity. A non-limiting example is a glucose oxidase (H2O2 producing) and glucose dehydrogenase (electrically coupled) configuration. Measurement of Glucose at two potentials and from two different electrodes provides more data points and accuracy. Such approaches may not be needed for glucose sensing, but the can be applied across the biomarker sensing spectrum of other analytes, alone or in combination with glucoses sensing, such as ketone sensing, ketone/lactate sensing, and ketone/glucose sensing.

In an alternative configuration of that depicted in FIGS. 8C-8D, two or more wire electrodes, which can be colinear, wrapped, or otherwise juxtaposed, are presented, where WE1 is separated from WE2, for example, from other elongated shaped electrode. Insulating layer electrically isolates WE1 from WE2. In this configuration, independent electrode potential can be applied to the corresponding electrode surfaces, where the independent electrode potential can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 8D, such an arrangement of RL's is depicted, where an additional RL 752′ is adjacent WES2 but substantially absent from WES1.

In one example of measuring two different analytes, the above configuration comprising enzyme domain EZL1 755 comprising one or more enzyme(s) and one or more mediators for at least one enzyme of EZL1 to provide for direct electron transfer to the WES1 and determining a concentration of at least a first analyte. In addition, enzyme domain EZL2 756 can comprise at least one enzyme that provides peroxide (e.g., hydrogen peroxide) or consumes oxygen during catalysis with its substrate. The peroxide or the oxygen produced in EZL2 756 migrates to WES2 and provides a detectable signal that corresponds directly or indirectly to a second analyte. For example, WES2 can be carbon, wired to glucose dehydrogenase to measure glucose, while WES1 can be platinum, that measures peroxided produced from lactate oxidase/lactate in EZL2 756. The combinations of electrode material and enzyme(s) as disclosed herein are examples and non-limiting.

In one example, the potentials of P1 and P2 can be separated by an amount of potential so that both signals (from direct electron transfer from EZL1 755 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2 can be correlated to transduced signal coming from either direct electron transfer or electrolysis of hydrogen peroxide, respectably. In one example, the modality of sensing is non-limiting and can include different amperometry techniques, e.g., cyclic voltammetry. In one example, an alternative configuration is provided but hydrogen peroxide production in EZL2 is replaced by another suitable electrolysis compound that maintains the P2 #P1 relationship, such as oxygen, and at least one enzyme-substrate combination that provide the other electrolysis compound.

For example, a continuous multi-analyte sensor configuration, for choline and glucose, in which enzyme domains EZ1 755, EZ2 756 were associated with different WEs, e.g., platinum WE2, and gold WE1 was prepared. In this exemplary case, EZL1 755 contained glucose oxidase and a mediator coupled to WE1 to facilitate electron direct transfer upon catalysis of glucose, and EZL2 756 contained choline oxidase that will catalyze choline and generate hydrogen peroxide for electrolysis at WE2. The EZL's were coated with resistance domains; upon cure and readiness they underwent cyclic voltammetry in the presence of glucose and choline. A wired glucose oxidase enzyme to a gold electrode is capable of transducing signal at 0.2 volts, therefore, by analyzing the current changes at 0.2 volts, the concentration of glucose can be determined. The data also demonstrates that choline concentration is also inferentially detectable at the WE2 platinum electrode if the CV trace is analyzed at the voltage P2.

In one example, either electrode WE1 or WE2 can be, for example, a composite material, for example a gold electrode with platinum ink deposited on top, a carbon/platinum mix, and or traces of carbon on top of platinum, or porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 8E, an example of such composite electrode surfaces is shown, in which an extended platinum covered wire 757 is half coated with carbon 758, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that may be used to form one or both of WE1 and WE2. In one example, a platinum-carbon electrode WE1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include ketone sensing (beta-hydroxybutyrate dehydrogenase electrically coupled enzyme in EZL1 755) and glucose sensing (glucose oxidase in EZL2 756). Other membranes can be used in the aforementioned configuration, such as electrode, resistance, bio-interfacing, and drug releasing membranes. In other examples, one or both of the working electrodes (WE1, WE2) may be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein may instead or additionally include graphene, graphene oxide, or other materials suitable for forming the working electrodes, such as commercially available carbon ink.

### Glycerol Sensor Configurations

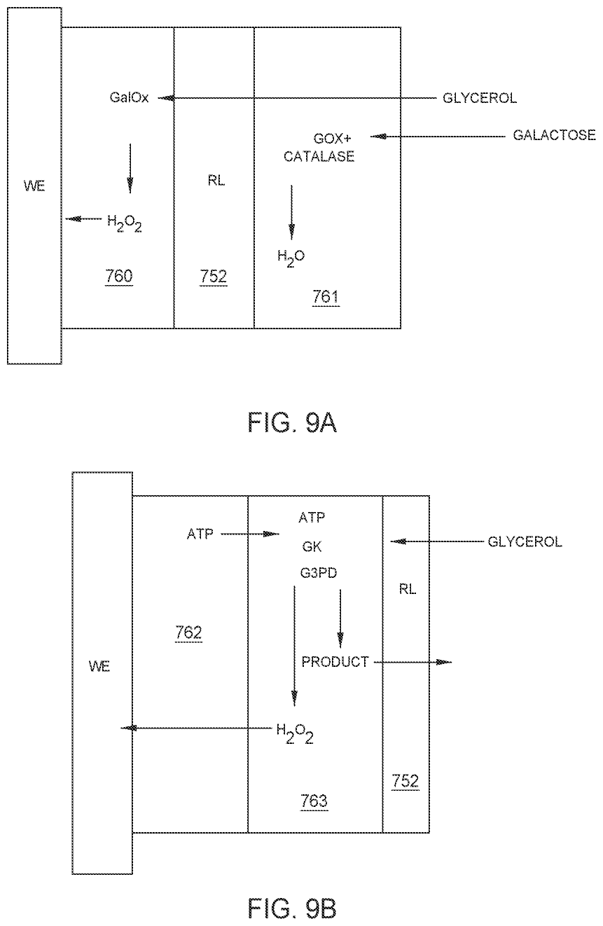

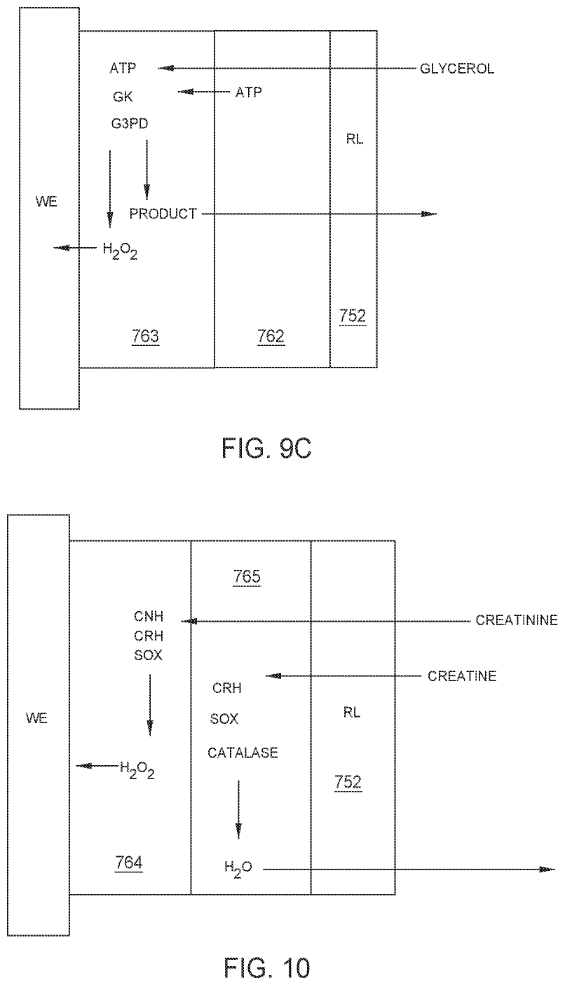

As shown in FIG. 9A, an exemplary continuous glycerol sensor configuration is depicted where a first enzyme domain EZL1 760 comprising galactose oxidase is positioned proximal to at least a portion of a WE surface. A second enzyme domain EZL2 761 comprising glucose oxidase and catalase is positioned more distal from the WE. As shown in FIG. 9A, one or more resistance domains (RL) 752 are positioned between EZL1 760 and EZL2 761. Additional RLs can be employed, for example, adjacent to EZL2 761. Modification of the one or more RL membranes to attenuate the flux of either analyte and increase glycerol to galactose sensitivity ratio is envisaged. The above glycerol sensing configuration provides for a glycerol sensor that can be combined with one or more additional sensor configurations as disclosed herein.

Glycerol can be catalyzed by the enzyme galactose oxidase (GalOx), however, GalOx has an activity ratio of 1%-5% towards glycerol. In one example, the activity of GalOx towards this secondary analyte glycerol can be utilized. The relative concentrations of glycerol in vivo are much higher that galactose (˜2 umol/l for galactose, and ˜100 umol/l for glycerol), which compliments the aforementioned configurations.

If the GalOx present in EZL1 760 membrane is not otherwise functionally limited, then the GalOx will catalyze most if not all of the glycerol that passes through the one or more RLs. The signal contribution from the glycerol present will be higher as compared to the signal contribution from galactose. In one example, the one or more RL's are chemically configured to provide a higher influx of glycerol or a lower influx of galactose.

In another example, a glycol sensor configuration is provided using multiple working electrodes WEs that provides for utilizing signal transduced from both WEs. Utilizing signal transduced from both WEs can provide increasing selectivity. In one example EZL1 760 and EZL2 761 comprise the same oxidase enzyme (e.g., galactose oxidase) with different ratios of enzyme loading, and/or a different immobilizing polymer and/or different number and layers of RL's over the WEs. Such configurations provide for measurement of the same target analyte with different sensitivities, resulting in a dual measurement. Using a mathematical algorithm to correct for noise and interference from a first signal, and inputting the first signal from one sensing electrode with a first analyte sensitivity ratio into the mathematical algorithm, allows for the decoupling of the second signal corresponding to the desired analyte contributions. Modification of the sensitivity ratio of the one or more EZL's to distinguish signals from the interfering species and the analyte(s) of interest can be provided by adjusting one or more of enzyme source, enzyme load in EZL's, chemical nature/diffusional characteristics of EZL's, chemical/diffusional characteristics of the at least one RL's, and combinations thereof.

As discussed herein, a secondary enzyme domain can be utilized to catalyze the non-target analyte(s), reducing their concentration and limiting diffusion towards the sensing electrode through adjacent membranes that contains the primary enzyme and necessary additives. In this example, the most distal enzyme domain, EZL2, 761 is configured to catalyze a non-target analyte that would otherwise react with EZL1, thus providing a potentially less accurate reading of the target analyte (glycerol) concentration. This secondary enzyme domain can act as a “selective diffusion exclusion membrane” by itself, or in some other configurations can be placed above or under a resistant layer (RL) 752. In this example, the target analyte is glycerol and GalOX is used to catalyze glycerol to form a measurable species (e.g., hydrogen peroxide).

In one example, a continuous glycerol sensor configuration is provided using at least glycerol oxidase, which provides hydrogen peroxide upon reaction and catalysis of glycerol. Thus, in one example, enzyme domain comprising glycerol oxidase can be positioned adjacent at least a portion of a WE surface and hydrogen peroxide is detected using amperometry. In another example, enzyme domain comprising glycerol oxidase is used for sensing oxygen level changes, for example, in a Clark type electrode setup. Alternatively, at least a portion of the WE surface can be coated with one more layers of electrically coupled polymers, such as a mediator system discussed below, to provide a coated WE capable of electron transfer from the enzyme at a lower potential. The coated WE can then operate at a different and lower voltage to measure oxygen and its correlation to glycerol concentration.

In another example, a glycerol sensor configuration is provided using glycerol-3-phosphate oxidase in the enzyme domain. In one example, ATP is used as the cofactor. Thus, as shown in FIGS. 9B and 9C, exemplary sensor configurations are depicted where in one example (FIG. 9B), one or more cofactors (e.g. ATP) 762 is proximal to at least a portion of an WE surface. One or more enzyme domains 763 comprising glycerol-3-phospohate oxidase (G3PD), lipase, and/or glycerol kinase (GK) and one or more regenerating enzymes capable of continuously regenerating the cofactor are contained in an enzyme domain are adjacent the cofactor, or more distal from the WE surface than the cofactor layer 762. Examples of regenerating enzymes that can be used to provide ATP regeneration include, but are not limited to, ATP synthase, pyruvate kinase, acetate kinase, and creatine kinase. The one or more regenerating enzymes can be included in one or more enzyme domains, or in a separate layer.

An alternative configuration is shown in FIG. 9C, where one or more enzyme domains 763 comprising G3PD, at least one cofactor and at least one regenerating enzyme, are positioned proximal to at least a portion of WE surface, with one or more cofactor reservoirs 762 adjacent to the enzyme domains comprising G3PD and more distal from the WE surface, and one or more RL's 752 are positioned adjacent the cofactor reservoir. In either of these configurations, an additional enzyme domain comprising lipase can be included to indirectly measure triglyceride, as the lipase will produce glycerol for detection by the aforementioned glycerol sensor configurations.

In another example, a glycerol sensor configuration is provided using dehydrogenase enzymes with cofactors and regenerating enzymes. In one example, cofactors that can be incorporated in the one or more enzyme domains include one or more of NAD(P)H, NADP+, and ATP. In one example, e.g., for use of NAD(P)H a regenerating enzyme can be NADH oxidase or diaphorase to convert NADH, the product of the dehydrogenase catalysis back to NAD(P)H. Similar methodologies can be used for creating other glycerol sensors, for example, glycerol dehydrogenase, combined with NADH oxidase or diaphorase can be configured to measure glycerol or oxygen.

In one example, mathematical modeling can be used to identify and remove interference signals, measuring very low analyte concentrations, signal error and noise reduction so as to improve and increase of multi-analyte sensor end of life. For example, with a two WE electrode configuration where WE1 is coated with a first EZL while WE2 is coated with two or more different EZL, optionally with one or more resistance domains (RL) a mathematical correction such interference can be corrected for, providing for increasing accuracy of the measurements.

Changes of enzyme load, immobilizing polymer and resistance domain characteristics over each analyte sensing region can result in different sensitive ratios between two or more target analyte and interfering species. If the signal are collected and analyzed using mathematical modeling, a more precise concentration of the target analytes can be calculated.

One example in which use of mathematical modeling can be helpful is with glycerol sensing, where galactose oxidase is sensitive towards both galactose and glycerol. The sensitivity ratio of galactose oxidase to glycerol is about is 1%-5% of its sensitivity to galactose. In such case, modification of the sensitivity ratio to the two analytes is possible by adjusting the one or more parameters, such as enzyme source, enzyme load, enzyme domain (EZL) diffusional characteristics, RL diffusional characteristics, and combinations thereof. If two WEs are operating in the sensor system, signal correction and analysis from both WEs using mathematical modeling provides high degree of fidelity and target analyte concentration measurement.

In the above configurations, the proximity to the WE of one or more of these enzyme immobilizing layers discussed herein can be different or reversed, for example if the most proximal to the WE enzyme domain provides hydrogen peroxide, this configuration can be used.

In some examples, the target analyte can be measured using one or multiple of enzyme working in concert. In one example, ATP can be immobilized in one or more EZL membranes, or can be added to an adjacent layer alone or in combination with a secondary cofactor, or can get regenerated/recycled for use in the same EZL or an adjacent third EZL. This configuration can further include a cofactor regenerator enzyme, e.g., alcohol dehydrogenase or NADH oxidase to regenerate NAD(P)H. Other examples of cofactor regenerator enzymes that can be used for ATP regeneration are ATP synthase, pyruvate kinase, acetate kinase, creatine kinase, and the like.

In one example, the aforementioned continuous glycerol sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations to provide a continuous multi-analyte sensor device as further described below. This continuous multi-analyte sensor device can further include continuous glucose monitoring capability. Other configurations can be used in the aforementioned continuous glycerol sensor configuration, such as electrode, resistance, bio-interfacing, and drug releasing membranes.

### Creatinine Sensor Configurations

In one example, continuous creatinine sensor configurations are provided, such configurations containing one or more enzymes and/or cofactors. Creatinine sensor configurations are examples of continuous analyte sensing systems that generate intermediate, interfering products, where these intermediates/interferents are also present in the biological fluids sampled. The present disclosure provides solutions to address these technical problems and provide for accurate, stable, and continuous creatinine monitoring alone or in combination with other continuous multi-analyte sensor configurations.

Creatinine sensors, when in use, are subject to changes of a number of physiologically present intermediate/interfering products, for example sarcosine and creatine, that can affect the correlation of the transduced signal with the creatinine concentration. The physiological concentration range of sarcosine, for example, is an order of magnitude lower that creatinine or creatine, so signal contribution from circulating sarcosine is typically minimal. However, changes in local physiological creatine concentration can affect the creatinine sensor signal. In one example, eliminating or reducing such signal contribution is provided.

Thus, in one example, eliminating or reducing creatine signal contribution of a creatinine sensor comprises using at least one enzyme that will consume the non-targeted interfering analyte, in this case, creatine. For example, two enzyme domains are used, positioned adjacent to each other. At least a portion of a first enzyme domain is positioned proximal to at least a portion of a WE surface, the first enzyme domain comprising one or more enzymes selected from creatinine amidohydrolase (CNH), creatine amidohydrolase (CRH), and sarcosine oxidase (SOX). A second enzyme domain, adjacent the first enzyme domain and more distal from the WE surface, comprises one or more enzymes using creatine as their substrate so as to eliminate or reduce creatine diffusion towards the WE. In one example, combinations of enzymes include CRH, SOX, creatine kinase, and catalase, where the enzyme ratios are tuned to provide ample number of units such that circulating creatine will at least partially be consumed by CRH providing sarcosine and urea, whereas the sarcosine produced will at least partially be consumed by SOX, providing an oxidized form of glycine (e.g. glycine aldehyde) which will at least be partially consumed by catalase. In an alternative configuration of the above, the urea produced by the CRH catalysis can at least partially be consumed by urease to provide ammonia, with the aqueous form (NH4+) being detected via an ion-selective electrode (e.g., nonactin ionophore). Such an alternative potentiometric sensing configuration may provide an alternative to amperometric peroxide detection (e.g., improved sensitivity, limits of detection, and lack of depletion of the reference electrode, alternate pathways/mechanisms). This dual-analyte-sensing example may include a creatinine-potassium sensor having potentiometric sensing at two different working electrodes. In this example, interference signals can be identified and corrected. In one alternative example, the aforementioned configuration can include multi-modal sensing architectures using a combination of amperometry and potentiometry to detect concentrations of peroxide and ammonium ion, measured using amperometry and potentiometry, respectively, and correlated to measure the concentration of the creatinine. In one example, the aforementioned configurations can further comprise one or more configurations (e.g., without enzyme) separating the two enzyme domains to provide complementary or assisting diffusional separations and barriers.

In yet another example, a method to isolate the signal and measure essentially only creatinine is to use a second WE that measures the interfering species (e.g., creatine) and then correct for the signal using mathematical modeling. Thus, for example, signal from the WE interacting with creatine is used as a reference signal. Signal from another WE interacting with creatinine is from corrected for signal from the WE interacting with creatine to selectively determine creatinine concentration.

In yet another example, sensing creatinine is provided by measuring oxygen level changes electrochemically, for example in a Clark type electrode setup, or using one or more electrodes coated with layers of different polymers such as NAFION™ and correlating changes of potential based on oxygen changes, which will indirectly correlate with the concentrations of creatinine.

In yet another example, sensing creatinine is provided by using sarcosine oxidase wired to at least one WE using one or more electrically coupled mediators. In this approach, concentration of creatinine will indirectly correlate with the electron transfer generated signal collected from the WE.

For the aforementioned creatinine sensor configurations based on hydrogen peroxide and/or oxygen measurements the one or more enzymes can be in a single enzyme domain, or the one or more enzymes, independently, can be in one or more enzyme domains, or any other combination thereof, in which in each layer at least one enzyme is present. For the aforementioned creatinine sensor configurations based on use of an electrically coupled sarcosine oxidase containing layer, the layer positioned adjacent to the electrode and is electrically coupled to at least a portion of the electrode surface using mediators.

In another example, the aforementioned creatinine sensor configurations can be sensed using potentiometry by using urease enzyme (UR) that creates ammonium from urea, the urea created by CRH from creatine, the creatine being formed from the interaction of creatinine with CNH. Thus, ammonium can be measured by the above configuration and correlated with the creatinine concentration. Alternatively, creatine amidohydrolase (CI) or creatinine deiminase can be used to create ammonia gas, which under physiological conditions of a transcutaneous sensor, would provide ammonium ion for signal transduction.

In yet another example, sensing creatinine is provided by using one or more enzymes and one or more cofactors. Some non-limiting examples of such configurations include creatinine deaminase (CD) providing ammonium from creatinine, glutamate dehydrogenase (GLDH) providing peroxide from the ammonium, where hydrogen peroxide correlates with levels of present creatinine. The above configuration can further include a third enzyme glutamate oxidase (GLOD) to further break down glutamate formed from the GDLH and create additional hydrogen peroxide. Such combinations of enzymes, independently, can be in one or more enzyme domains, or any other combination thereof, in which in each domain or layer, at least one enzyme is present.

In yet another example, sensing creatinine is provided by the combination of creatinine amidohydrolase (CNH), creatine kinase (CK) and pyruvate kinase (PK), where pyruvate, created by PK can be detected by one or more of either lactate dehydrogenase (LDH) or pyruvate oxidase (POX) enzymes configured independently, where one or more of the aforementioned enzyme are present in one layer, or, in which in each of a plurality of layers comprises at least one enzyme, any other combination thereof.

In such sensor configurations where one or more cofactors and/or regenerating enzymes for the cofactors are used, providing excess amounts of one or more of NADH, NAD(P)H and ATP in any of the one or more configurations can be employed, and one or more diffusion resistance domains can be introduced to limit or prevent flux of the cofactors from their respective membrane(s). Other configurations can be used in the aforementioned configurations, such as electrode, resistance, bio-interfacing, and drug releasing membranes.

In yet another example, creatinine detection is provided by using creatinine deiminase in one or more enzyme domains and providing ammonium to the enzyme domain(s) via catalysis of creatinine. Ammonium ion can then be detected potentiometrically or by using composite electrodes that undergo redox when exposed to ammonium ion, for example NAFION™/polyaniline composite electrodes, in which polyaniline undergoes redox in the presence of ammonium at the electrode under potential. Ammonium concentration can then be correlated to creatinine concentration.

FIG. 10 depicts an exemplary continuous sensor configuration for creatinine. In the example of FIG. 10, the sensor includes a first enzyme domain 764 comprising CNH, CRH, and SOX are adjacent a working electrode WE, e.g., platinum. A second enzyme domain 765 is positioned adjacent the first enzyme domain and is more distal from the WE. One or more resistance domains (RL) 752 can be positioned adjacent the second enzyme domain or between the first and second layers. Creatinine is diffusible through the RL and the second enzyme domain to the first enzyme domain where it is converted to peroxide and transduces a signal corresponding to its concentration. Creatine is diffusible through the RL and is converted in the second enzyme domain to sarcosine and urea, the sarcosine being consumed by the sarcosine oxidase and the peroxide generated is consumed by the catalase, thus preventing transduction of the creatine signal.

For example, variations of the above configuration are possible for continuous monitoring of creatinine alone or in combination with one or more other analytes. Thus, one alternative approach to sensing creatinine could be sensing oxygen level changes electrochemically, for example in a Clark-type electrode setup. In one example, the WE can be coated with layers of different polymers, such as NAFION™ and based on changes of potential oxygen changes, the concentrations of creatinine can be correlated. In yet another example, one or more enzyme most proximal to the WE, i.e., sarcosine oxidase, can be “wired” to the electrode using one or more mediators. Each of the different enzymes in the above configurations can be distributed inside a polymer matrix or domain to provide one enzyme domain. In another example, one or more of the different enzymes discussed herein can be formed as the enzyme domain and can be formed layer by layer, in which each layer has at least one enzyme present. In an example of a “wired” enzyme configuration with a multilayered membrane, the wired enzyme domain would be most proximal to the electrode. One or more interferent layers can be deposited among the multilayer enzyme configuration so as to block of non-targeted analytes from reaching electrodes.

In one example, the aforementioned continuous creatinine sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations to provide a continuous multi-analyte sensor device as further described below. This continuous multi-analyte sensor device can further include continuous glucose monitoring capability.

### Lactose Sensor Configurations

In one example, a continuous lactose sensor configuration, alone or in combination with another analyte sensing configuration comprising one or more enzymes and/or cofactors is provided. In a general sense, a lactose sensing configuration using at least one enzyme domain comprising lactase enzyme is used for producing glucose and galactose from the lactose. The produced glucose or galactose is then enzymatically converted to a peroxide for signal transduction at an electrode. Thus, in one example, at least one enzyme domain EZL1 comprising lactase is positioned proximal to at least a portion of a WE surface capable of electrolysis of hydrogen peroxide. In one example, glucose oxidase enzyme (GOX) is included in EZL1, with one or more cofactors or electrically coupled mediators. In another example, galactose oxidase enzyme (GalOx) is included in EZL1, optionally with one or more cofactors or mediators. In one example, glucose oxidase enzyme and galactose oxidase are both included in EZL1. In one example, glucose oxidase enzyme and galactose oxidase are both included in EZL1, optionally with one or more cofactors or electrically coupled mediators.

One or more additional EZL's (e.g. EZL2) can be positioned adjacent the EZL1, where at least a portion of EZL2 is more distal from at least a portion of WE than EZL1. In one example, one or more layers can be positioned in between EZL1 and EZL2, such layers can comprise enzyme, cofactor or mediator or be essentially devoid of one or more of enzymes, cofactors or mediators. In one example, the one or more layers positioned in between EZL1 and EZL2 is essentially devoid of enzyme, e.g., no purposefully added enzyme. In one example one or layers can be positioned adjacent EZL2, being more distal from at least a portion of EZL1 than EZL2, and comprise one or more of the enzymes present in either EZL1 or EZL2.

In one example of the aforementioned lactose sensor configurations, the peroxide generating enzyme can be electrically coupled to the electrode using coupling mediators. The transduced peroxide signals from the aforementioned lactose sensor configurations can be correlated with the level of lactose present.

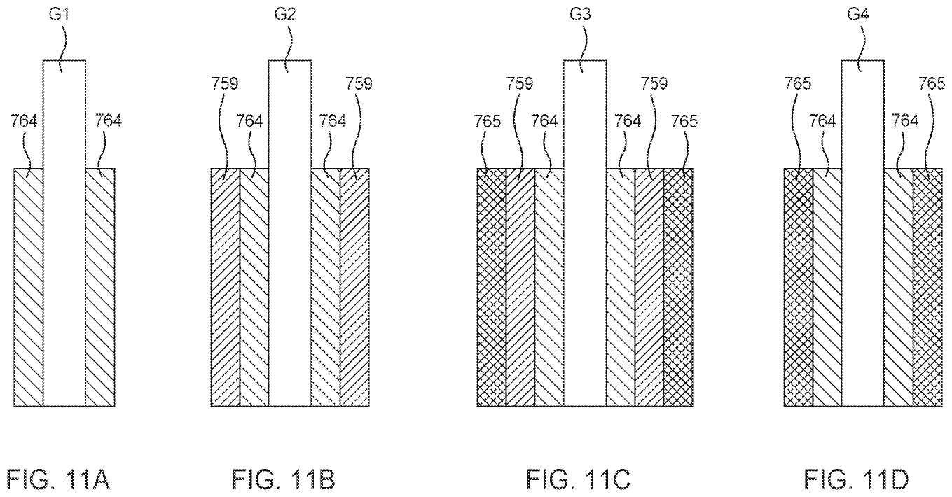

FIG. 11A-11D depict alternative continuous lactose sensor configurations. Thus, in an enzyme domain EZL1 764 most proximal to WE (G1), comprising GalOx and lactase, provides a lactose sensor that is sensitive to galactose and lactose concentration changes and is essentially non-transducing of glucose concentration. As shown in FIGS. 11B-11D, additional layers, including non-enzyme containing layers 759, and an enzyme domain 765 (e.g., a lactase enzyme containing layer), and optionally, electrode, resistance, bio-interfacing, and drug releasing membranes. (not shown) are used. Since changes in physiological galactose concentration are minimal, the transduced signal would essentially be from physiological lactose fluctuations.

In one example, the aforementioned continuous lactose sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations, creatinine sensor configurations to provide a continuous multi-analyte sensor device as further described below. This continuous multi-analyte sensor device can further include continuous glucose monitoring capability. Other membranes can be used in the aforementioned sensor configuration, such as electrode, resistance, bio-interfacing, and drug releasing membranes.

### Urea Sensor Configurations

Similar approach as described above can also be used to create a continuous urea sensor. For example urease (UR), which can break down the urea and to provide ammonium can be used in an enzyme domain configuration. Ammonium can be detected with potentiometry or by using a composite electrodes, e.g., electrodes that undergo redox when exposed to ammonium. Example electrodes for ammonium signal transduction include, but are not limited to, NAFION™/polyaniline composite electrodes, in which polyaniline undergoes redox in the presence of ammonium at an applied potential, with essentially direct correlation of signal to the level of ammonium present in the surrounding. This method can also be used to measure other analytes such as glutamate using the enzyme glutaminase (GLUS).

In one example, the aforementioned continuous uric acid sensor configurations can be combined with any one of the aforementioned continuous alcohol sensor configurations and/or continuous uric acid sensor configurations and/or continuous cholesterol sensor configurations and/or continuous bilirubin/ascorbic acid sensor configurations and/or continuous ketone sensor configurations and/or continuous choline sensor configurations and/or continuous glycerol sensor configurations and/or continuous creatinine sensor configurations and/or continuous lactose sensor configurations to provide a continuous multi-analyte sensor device as further described below. This continuous multi-analyte sensor device can further include continuous glucose monitoring capability. Other membranes can be used in the aforementioned uric acid sensor configuration, such as electrode, resistance, bio-interfacing, and drug releasing membranes.

In certain embodiments, continuous analyte monitoring system 104 may be a potassium sensor, as discussed in reference to FIG. 1. FIGS. 7-14 describe an example sensor device used to measure an electrophysiological signal and/or concentration of a target analyte (e.g., potassium), according to certain embodiments of the present disclosure.

The term “ion” as used herein is a broad term, and is to be given its ordinary and customary meaning to a person of ordinary skill in the art (and is not to be limited to a special or customized meaning), and refers without limitation to an atom or molecule with a net electric charge due to the loss or gain of one or more electrons. Ions in a biological fluid may be referred to as “electrolytes.” Nonlimiting examples of ions in biological fluids include sodium (Na⁺), potassium (K⁺), magnesium (Mg²⁺), calcium (Ca²⁺), hydrogen (H⁺), lithium (Li⁺), chloride (Cl⁻), sulfide (S²⁻), sulfite (SO₃²⁻), sulfate (SO₄²⁻), phosphate (PO₄³⁻), and ammonium (NH₄⁺). An ion is an example of an analyte.

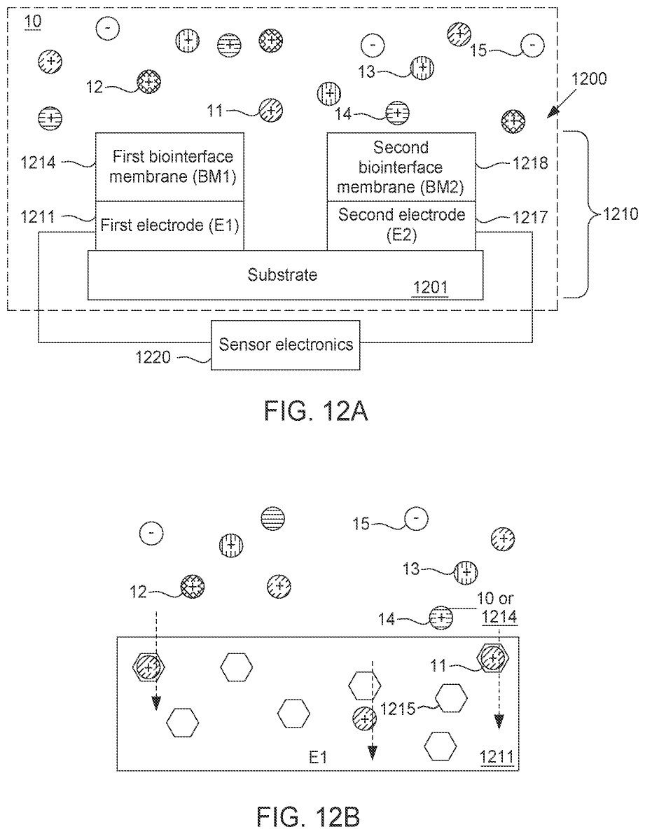

FIG. 12A schematically illustrates an example configuration and component of a device 1200 for measuring an electrophysiological signal and/or concentration of a target analyte such as a target ion 11 in a biological fluid 10 in vivo. Turning first to FIG. 12, device 1200 includes indwelling sensor 1210 and sensor electronics 1220. Sensor 1210 includes substrate 1201, first electrode (E1) 1211 disposed on the substrate, and a second electrode (E2) 1217 disposed on the substrate. First electrode 1211 may be referred to as a working electrode (WE), while second electrode 1217 may be referred to as a reference electrode (RE). The sensor electronics 1220 may be configured to generate a signal corresponding to an electromotive force (EMF). In some examples, the EMF is at least partially based on a potential difference that is generated between the first electrode 1211 and the second electrode 1217 responsive to biological fluid 10 conducting the electrophysiological signal to first electrode 1211. Sensor electronics 1220 may be configured to use the signal to generate an output corresponding to a measurement of the signal. In various examples, the EMF is at least partially based on a potential difference between (i) either the first electrode 1211 or the second electrode 1217 and (ii) another electrode which is spaced apart from the first electrode or second electrode.

Additionally, or alternatively, in some examples, device 1200 may include an ionophore, such as ionophore 1215 as shown in FIG. 12B, disposed on the substrate 1201 and configured to selectively transport the target ion 11 to or within the first electrode 1211. The EMF may be at least partially based on a potential difference may be generated between the first electrode 1211 and the second electrode 1217 responsive to the ionophore transporting the target ion to or through the first electrode 1211. The sensor electronics 1220 (and/or an external device that receives the signal via a suitable wired or wireless connection) may be configured to use the signal to generate an output corresponding to a measurement of the concentration of the target ion in the biological fluid. Further details regarding the configuration and use of sensor electronics 1220 are provided further below.

Optionally, the first electrode 1211 may be used to measure an electrophysiological signal in addition to ion concentration. In other examples, such as when device 1200 is configured to detect an electrophysiological signal but not an ion concentration, first electrode 1211 need not include an ionophore, such as ionophore 1215 as shown in FIG. 12B. In other examples, the first electrode 1211 may include an ionophore that is inactive such that it does not interfere with the measurement of the electrophysiological signal.

In a manner such as illustrated in FIG. 12A, biological fluid 10 may include a plurality of ions 11, 12, 13, 14, and 15. Device 1200 may be configured to measure the concentration of ion 11, and accordingly such ion may be referred to as a “target” ion. Target ion 11 may be any suitable ion, and in nonlimiting examples is selected from the group consisting of sodium (Na⁺), potassium (K⁺), magnesium (Mg²⁺), calcium (Ca²⁺), hydrogen (H⁺), lithium (Li⁺), chloride (Cl⁻), sulfite (SO₃²⁻), sulfate (SO₄²⁻), phosphate (PO₄³⁻), and ammonium (NH₄⁺). Ions 12, 13, 14, and 15 may be others of the group consisting of sodium (Na⁺), potassium (K⁺), magnesium (Mg²⁺), calcium (Ca²⁺), hydrogen (H⁺), lithium (Li⁺), chloride (Cl⁻), sulfide (S²⁻), sulfite (SO₃²⁻), sulfate (SO₄²⁻), phosphate (PO₄³⁻), and ammonium (NH₄⁺). Ions 12, 13, 14, and 15 may be considered interferants to the measurement of target ion 11 because they have the potential interfere with the measurement of target ion 11 by sensor to produce a signal that does not accurately represent the concentration of target ion 11. Ionophore, such as ionophore 1215 as shown in FIG. 12B, may be selected so as to selectively transport target ion 11 to or within first electrode 1211 and to inhibit, fully, partially and/or substantially, the transport of one or more of ions 12, 13, 14, or 15 to or within first electrode 1211. For example, as illustrated in FIG. 12B, ionophore 1215 may selectively transport, or selectively bind, target ions 11 from biological fluid 10 or from biointerface membrane 1214 (if provided, e.g., as described below) to and within first electrode 1211, while ions 12, 13, 14, and 15 may substantially remain within biological fluid 10 or biointerface membrane 1214. Accordingly, contributions to the potential difference between first electrode 1211 and second electrode 1217 responsive to the transport of ions to or within first electrode 1211 substantially may be primarily caused by target ion 11 instead of by one or more of ions 12, 13, 14, or 15.

A wide variety of ionophores 1215 may be used to selectively transport corresponding ions in a manner such as described with reference to FIGS. 12A-12B. For example, where the target ion 11 is hydrogen (via peroxide), the ionophore 1215 may be tridodecylamine, 4-nonadecylpyridine, N,N-dioctadecylmethylamine, octadecyl isonicotinate, calix[4]-aza-crown. Or, for example, where the target ion 11 is lithium, the ionophore 1215 may be ETH 149, N,N,N′,N′,N″,N″-hexacyclohexyl-4,4′,4″-propylidynetris(3-oxabutyramide), or 6,6-Dibenzyl-1,4,8-11-tetraoxacyclotetradecane. Or, for example, where the target ion 11 is sulfite, the ionophore 1215 may be octadecyl 4-formylbenzoate. Or, for example, where the target ion 11 is sulfate, the ionophore 1215 may be 1,3-[bis(3-phenylthioureidomethyl)]benzene or zinc phthalocyanine. Or, for example, where the target ion 11 is phosphate, the ionophore 1215 may be 9-decyl-1,4,7-triazacyclodecane-8,10-dione. Or, for example, where the target ion 11 is sodium, the ionophore 1215 may be 4-tert-butylcalix[4]arene-tetraacetic acid tetraethyl ester (sodium ionophore X) or calix[4]arene-25,26,27,28-tetrol (calix[4]arene). Or, for example, where the target ion 11 is potassium, the ionophore 1215 may be potassium ionophore II (BB15C5) or valinomycin. Or, for example, where the target ion 11 is magnesium, the ionophore 1215 may be 4,5-bis(benzoylthio)-1,3-dithiole-2-thione (Bz2dmit) or 1,3,5-Tris[10-(1-adamantyl)-7,9-dioxo-6,10-diazaundecyl]benzene (magnesium ionophore VI). Or, for example, where the target ion 11 is calcium, the ionophore 1215 may be calcium ionophore I (ETH 1001) or calcium ionophore II (ETH129). Or, for example, where the target ion 11 is chloride, the ionophore 1215 may be tridodecylmethylammonium chloride (TDMAC). Or, for example, where the target ion 11 is ammonium, the ionophore 1215 may be nonactin.

In the nonlimiting example illustrated in FIG. 12A, ionophore 1215 may be provided within first electrode 1211, and in such example the first electrode may be referred to as an ion-selective electrode (ISE), since the ionophore 1215 selectively transports the target ion 11. In some examples, first electrode 1211 may include a conductive polymer optionally having ionophore 1215 therein. Illustratively, the conductive polymer may be present in an amount of about 90 to about 99.5 weight percent in the first electrode 1211. The ionophore 1215 may be present in an amount of about 0.5 to about 10 weight percent in the first electrode. In some examples, the conductive polymer may be selected from the group consisting of: poly(3,4-ethylenedioxythiophene) (PEDOT), poly(3,4-ethylenedioxythiophene) polystyrene sulfonate (PEDOT:PSS), polyaniline (PANI), poly(pyrrole) (PPy), or poly(3-octylthiophene) (POT).

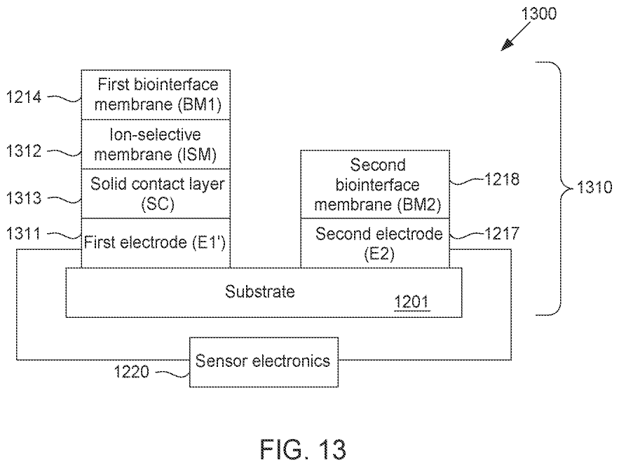

While conductive polymers (such as listed above) suitably may be used in a first electrode 1211 that excludes ionophore 1215, other materials alternatively may be used, some nonlimiting examples of which are described below with reference to FIG. 13. Optionally, ionophore 1215 may be provided in a membrane which is disposed on a first electrode 1211 (which electrode may exclude ionophore 1215), e.g., such as will be described below with reference to FIG. 13.

First electrode 1211 may be configured in such a manner as to enhance its biocompatibility. For example, first electrode 1211 may substantially exclude any plasticizer, which otherwise may leach into the biological fluid 10, potentially causing toxicity and/or a degradation in device performance. As used herein, the “substantial” exclusion of materials such as plasticizers is intended to mean that the first electrode 1211 or other aspects discussed herein do not contain detectable quantities of the “substantially” excluded material. In some examples, the first electrode 1211 may consist essentially of the conductive polymer, optionally in addition to the ionophore 1215. In some examples, the first electrode 1211 may consist essentially of the conductive polymer, the ionophore 1215, and an additive with ion exchanger capability. Such an additive may act as an ion exchanger. In one example, the additive contributes to the ion selectivity. In another example, the additive may not provide ion selectivity. For example, the additive may help to provide a substantially even concentration of the ion in the membrane. Additionally, or alternatively, the additive may help any change in ion concentration in the biofluid to cause an ion exchange within the membrane that may induce a non-selective potential difference. Additionally, or alternatively, the ionophore and the ion exchanger may form a complex which improves the ionophore's selectivity towards the target ion as compared to the selectivity of the ionophore alone.

Optionally, the additive may include a lipophilic salt. In nonlimiting examples, the lipophilic salt is selected from the group consisting of sodium tetrakis[3,5-bis(trifluoromethyl)phenyl]borate (NaTPFB), sodium tetraphenylborate (NaTPB), potassium tetrakis [3,5-bis(trifluoromethyl)phenyl]borate (KTFPB), and potassium tetrakis(4-chlorophenyl)borate (KTCIPB). The additive may be present in an amount of about 0.01 to about 1 weight percent in the first electrode, or other suitable amount.

Other materials within sensor 1210 may be selected. For example, substrate 1201 may include a material selected from the group consisting of: metal, glass, transparent conductive oxide, semiconductor, dielectric, ceramic, and polymer (such as biopolymer or synthetic polymer). In some examples, second electrode 1317 may include a metal, a metal alloy, a transition metal oxide, a transparent conductive oxide, a carbon material, a doped semiconductor, a binary semiconductor, a ternary semiconductor, or a conductive polymer. The binary semiconductor may include any two elements suitable for use in a semiconductor. The ternary semiconductor may include two or more binary semiconductors. In examples where a metal or a metal alloy is used, the metal or metals used can be selected from the group consisting of: gold, platinum, silver, iridium, rhodium, ruthenium, nickel, chromium, and titanium. The metal optionally may be oxidized or optionally may be in the form of a metal salt. A nonlimiting example of an oxidized metal which may be used in second electrode 1217 is iridium oxide. The carbon material may be selected from the group consisting of: carbon paste, graphene oxide, carbon nanotubes, C60, porous carbon nanomaterial, mesoporous carbon, glassy carbon, hybrid carbon nanomaterial, graphite, and doped diamond. The doped semiconductor may be selected from the group consisting of: silicon, germanium, silicon-germanium, zinc oxide, gallium arsenide, indium phosphide, gallium nitride, cadmium telluride, indium gallium arsenide, and aluminum arsenide. The transition metal oxide may be selected from the group of: titanium dioxide (TiO₂), iridium dioxide (IrO₂), platinum dioxide (PtO₂), zinc oxide (ZnO), copper oxide (CuO), cerium dioxide (CeO₂), ruthenium(IV) oxide (RuO₂), tantalum pentoxide (Ta₂O₅), titanium dioxide (TiO₂), molybdenum dioxide (MoO₂), and manganese dioxide (MnO₂). The metal alloy may be selected from the group consisting of: platinum-iridium (Pt—Ir), platinum-silver (Pt—Ag), platinum-gold (Pt—Au), gold-iridium (Au—Ir), gold-copper (Au—Cu), gold-silver (Au—Ag), and cobalt-iron (Co—Fe).

The conductive polymer that may be used for the sensor 2010 may be selected from the group consisting of: poly(3,4-ethylenedioxythiophene) (PEDOT), poly(3,4-ethylenedioxythiophene) polystyrene sulfonate (PEDOT:PSS), polyaniline (PANI), poly(pyrrole) (PPy), or poly(3-octylthiophene) (POT). That is, first electrode 1311 and second electrode 1317 optionally may be formed of the same material as one another, or may be formed using different materials than one another. In the nonlimiting example illustrated in FIG. 12A, first electrode 1211 and second electrode 1217 may be disposed directly on substrate 1201, or alternatively may be disposed on substrate 1201 via one or more intervening layers (not illustrated).

The biocompatibility of sensor 1210 optionally may be further enhanced by providing a biointerface membrane over one or more component(s) of sensor 1210. For example, in the nonlimiting configuration illustrated in FIG. 12A, a first biointerface membrane (BM1) 1214 may be disposed on the ionophore 1215 and the first electrode 1211. In another example, the first biointerface membrane (BM1) 1214 may be disposed on the ionophore 1215 and the first electrode 1211, and a second biointerface membrane (BM2) 1218 may be disposed on the second electrode 1217. Although FIG. 12A may suggest that the biointerface membrane(s) have a rectangular shape for simplicity of illustration, it should be apparent that the membrane(s) may conform to the shape of any underlying layers. In some examples, the biointerface membrane(s) may be configured to inhibit biofouling of the ionophore 1215, the first electrode 1211, and/or the second electrode 1217. Nonlimiting examples of materials which may be included in the biointerface membrane(s) include hard segments and/or soft segments. Examples of hard and soft segments used for the biointerface membrane 1214/1214′/1218 or other biointerface membranes as discussed herein include aromatic polyurethane hard segments with Si groups, aliphatic hard segments, polycarbonate soft segments or any combination thereof. In other examples of biointerface membrane(s) such as 1214/1214′/1218 or other biointerface membranes discussed herein, PVP may not be included. In this example where no PVP is included, the biointerface membrane (1218, 1214, 1214′, or other biointerface membranes as discussed herein) may include polyurethane and PDMS. In some examples, which may be combined with other examples herein, the biointerface membranes discussed herein may include one or more zwitterionic compounds.

Whereas ionophore 1215 is included within first electrode 1211 in the example described with reference to FIG. 12A, in the example illustrated in FIG. 13 first electrode 1311 does not include ionophore 1215 (and thus may be referred to as E1′ rather than E1). Instead, ionophore 1215 may be within an ion-selective membrane (ISM) 1312 disposed on the first electrode 1311. Ionophores 1215 may selectively transport target ion 11 to first electrode 1311 in a manner similar to that described with reference to FIGS. 12A-12B, and such transport may cause a potential difference between the first electrode 1311 and second electrode 1217 based upon which sensor electronics 1220 may generate an output corresponding to a measurement of the concentration of target ion 11 in biological fluid 10. It will be appreciated that in examples in which device 1200 is used to measure an electrophysiological signal and is not used to measure an ion concentration, ISM 1312 may be omitted.

In a manner similar to that described with reference to first electrode 1211, ion-selective membrane 1312 substantially may exclude any plasticizer. In some examples, ion-selective membrane 1312 may consist essentially of a biocompatible polymer and ionophore 1215 configured to selectively bind the target ion. Alternatively, in some examples, the ion-selective membrane 1312 may consist essentially of a biocompatible polymer, an ionophore 1215 configured to selectively bind the target ion 11, and an additive with ion exchanger capability, such as a lipophilic salt. Nonlimiting examples of lipophilic salts, and nonlimiting amounts of additives, biocompatible polymers, and ionophores are provided above with reference to FIGS. 12A-12B. Whereas first electrode 1211 includes a conductive polymer so as to be able to provide ionophore 1215 therein while retaining the electrical conductivity of an electrode, additional types of materials may be used in ion-selective membrane 1312 because the ion-selective membrane 1312 need not be used as an electrode. For example, the biocompatible polymer of the ion-selective membrane 1312 may include a hydrophobic polymer. Illustratively, the hydrophobic polymer may be selected from the group consisting of silicone, fluorosilicone (FS), polyurethane, polyurethaneurea, polyurea. In one example, the biocompatible polymer of the ISM 1412 (or other ion-selective membranes or other membranes discussed here) may include one or more block copolymers, which may be segmented block copolymers. In one example, the hydrophobic polymer may be a segmented block copolymer comprising polyurethane and/or polyurea segments, and/or polyester segments, and one or more of polycarbonate, polydimethylsiloxane (PDMS), methylene diphenyl diisocyanate (MDI), polysulfone (PSF), methyl methacrylate (MMA), poly(ε-caprolactone) (PCL), and 1,4-butanediol (BD). In other examples, the hydrophobic polymer may alternately or additionally include poly(vinyl chloride) (PVC), fluoropolymer, polyacrylate, and/or polymethacrylate.

In one example, the biocompatible polymer may include a hydrophilic block copolymer instead of or in addition to one or more hydrophobic copolymers. Illustratively, the hydrophilic block copolymer may include one or more hydrophilic blocks selected from the group consisting of polyethylene glycol (PEG) and cellulosic polymers. Additionally, or alternatively, the block copolymer may include one or more hydrophobic blocks selected from the group consisting of polydimethylsiloxane (PDMS) polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, poly(propylene oxide) and copolymers and blends thereof. In one example, the ion-selective membrane 2112 does not contain PVP, or other plasticizers.

In one example, the biocompatible polymer of the ion-selective membrane 1312 includes from about 0.1 wt. % silicone to about 80 wt. % silicone. In one example, the ion-selective membrane 1312, or other ion-selective membranes discussed herein, includes from about 5 wt. % silicone to about 25 wt. % silicone. In yet another example, the ion-selective membrane 1312, or other ion-selective membranes discussed herein, includes from about 35 wt. % silicone to about 65 wt. % silicone. In yet another example, the ion-selective membrane 1312, or other ion-selective membranes discussed herein, includes from about 30 wt. % silicone to about 50 wt. % silicone.

In certain examples, the ISM 1312 or other ISMs discussed herein may include one or more block copolymers or segmented block copolymers. The segmented block copolymer may include hard segments and soft segments. In this example, the hard segments may include aromatic or aliphatic diisocyanates are used to prepare hard segments of segmented block copolymer. In one example, the aliphatic or aromatic diisocyanate used to provide hard segment of polymer includes one or more of norbornane diisocyanate (NBDI), isophorone diisocyanate (IPDI), tolylene diisocyanate (TDI), 1,3-phenylene diisocyanate (MPDI), trans-1,3-bis(isocyanatomethyl) cyclohexane (1,3-H6XDI), bicyclohexylmethane-4,4′-diisocyanate (HMDI), 4,4′-diphenylmethane diisocyanate (MDI), trans-1,4-bis(isocyanatomethyl) cyclohexane (1,4-H6XDI), 1,4-cyclohexyl diisocyanate (CHDI), 1,4-phenylene diisocyanate (PPDI), 3,3′-Dimethyl-4,4′-biphenyldiisocyanate (TODI), 1,6-hexamethylene diisocyanate (HDI), or combinations thereof. In one example, the hard segments may be from about 5 wt. % to about 90 wt. % of the segmented block copolymer of the ISM 1312. In another example, the hard segments may be from about 15 wt. % to about 75 wt. %. In yet another example, the hard segments may be from about 25 wt. % to about 55 wt. %. It will be appreciated that ion-selective membrane 1312 and first electrode 1211 may be prepared in any suitable manner. Illustratively, the polymer, ionophore 1215, and any additive may be dispersed in appropriate amounts in a suitable organic solvent (e.g., tetrahydrofuran, isopropyl alcohol, acetone, or methyl ethyl ketone). The mixture may be coated onto substrate 1201 (or onto a layer thereon) using any suitable technique, such as dipping and drying, spray-coating, inkjet printing, aerosol jet dispensing, slot-coating, electrodeposition, electrospraying, electrospinning, chemical vapor deposition, plasma polymerization, physical vapor deposition, spin-coating, or the like. The organic solvent may be removed so as to form a solid material corresponding to ion-selective membrane 1312 or first electrode 1211. Other layers in device 1200 or device 1300, such as electrodes, solid contact layers, and/or biological membranes, may be formed using techniques described elsewhere herein or otherwise known in the art.

Whereas first electrode 1211 includes a conductive polymer so as to be able to provide ionophore 1215 therein while retaining the electrical conductivity of an electrode, additional types of materials may be used in first electrode 1311 because an ionophore need not be provided therein. Nonlimiting example materials for use in first electrode 1311 of device 1300 are provided above with reference to second electrode 1217, e.g., a metal, a metal alloy, a transition metal oxide, a transparent conductive oxide, a carbon material, a doped semiconductor, a binary semiconductor, a ternary semiconductor, or a conductive polymer such as described above with reference to FIG. 12A.

In some examples, the ion-selective membrane is in direct contact with the first electrode. In other examples, such as illustrated in FIG. 13, sensor 1310 further may include a solid contact layer 1313 disposed between the first electrode 1311 and the ion-selective membrane 1312. Solid contact layer 1313 may perform the function of enhancing the reproducibility and stability of the EMF by converting the signal into a measurable electrical potential signal. Additionally, or alternatively, solid contact layer 1313 may inhibit transport of water from the biological fluid 10 to the first electrode 1311 and/or accumulation of water at the first electrode 1311. Solid contact layer 1313 may include any suitable material or combination of materials. Nonlimiting example materials for use in solid contact layer 1313 are provided above with reference to second electrode 1217, e.g., a metal, a carbon material, a doped semiconductor, or a conductive polymer such as described above with reference to FIG. 12A. Alternatively, solid contact layer 1313 may include a redox couple which has a well-controlled concentration ratio of oxidized/reduced species that may be used to stabilize the interfacial electrical potential. The redox couple may include metallic centers with different oxidation states. Illustratively, the metallic centers may be selected from the group consisting of Co(II) and Co(III); Ir(II) and Ir(III); and Os(II) and Os(III). In alternative examples, the solid contact layer 213 may include a mixed conductor, or mixed ion-electron conductor, such as strontium titanate (SrTiO₃), titanium dioxide (TiO₂), (La,Ba,Sr)(Mn,Fe,Co)O_{3-d},La₂CuO_{4+d}, cerium(IV) oxide (CeO₂), lithium iron phosphate (LiFePO₄), and LiMnPO₄.

It will further be appreciated that sensor 1310 may have any suitable configuration. In the nonlimiting example illustrated in FIG. 13, substrate 1201 may be planar or substantially planar.

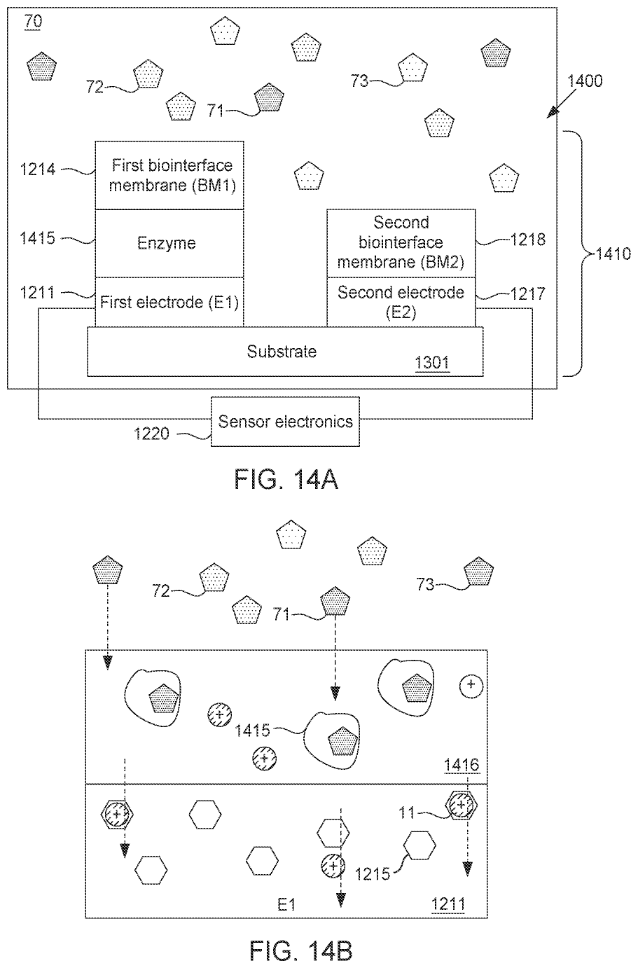

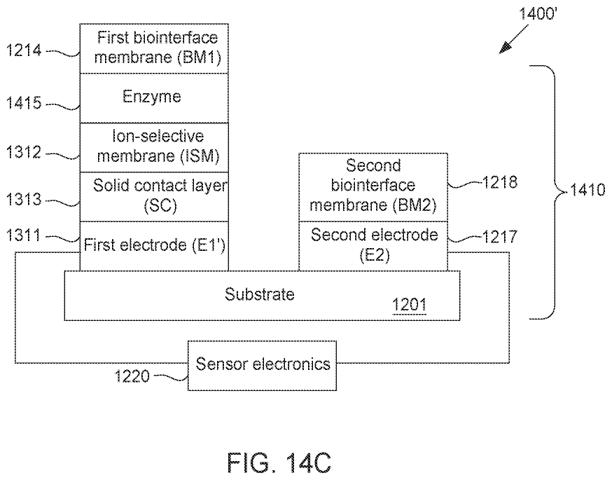

In the nonlimiting example illustrated in FIG. 14A, the ionophore may be located within first electrode (E1) 1211 disposed on the substrate and may be configured similarly as described with reference to FIG. 12A. Alternatively, in the nonlimiting example illustrated in FIG. 14C, the ionophore may be located within ion-selective membrane 1312 which may be configured in a manner such as described with reference to FIG. 13, and the first electrode 1311 may be configured in a manner such as described with reference to FIG. 13. First electrode 1211 or 1311 may be referred to as a working electrode (WE), while second electrode 1217 may be referred to as a reference electrode (RE).

The sensor electronics 1220 may be configured to generate a signal corresponding to an electromotive force (EMF). In some examples, the EMF is at least partially based on a potential difference that is generated between the first electrode and the second electrode responsive to the ionophore transporting the target ion to the first electrode. The sensor electronics 1220 may be configured to use the signal to generate an output corresponding to a measurement of the concentration of the target ion in the biological fluid, and/or may be configured to transmit the signal to an external device configured to use the signal to generate an output corresponding to a measurement of the concentration of the target ion in the biological fluid. Optionally, in some examples, the EMF is at least partially based on a potential difference that is generated between the first electrode and the second electrode responsive to biological fluid 10 conducting the electrophysiological signal to first electrode 111, and sensor electronics 1220 may be configured to use the signal to generate an output corresponding to a measurement of the electrophysiological signal.

In a manner such as illustrated in FIG. 14A, biological fluid 10 may include a plurality of analytes 71, 72, and 73. Device 1400 may be configured to measure the concentration of analyte 71, and accordingly such analyte may be referred to as a “target” analyte. As illustrated in FIG. 14B, enzyme 1415 may be located within enzyme layer 1416, and may selectively act upon target analyte 71 from biological fluid 10 or from biointerface membrane 1214 (if provided, e.g., as illustrated in FIG. 14A and configured similarly as described with reference to FIGS. 12A and 13). The action of enzyme 1415 upon the target analyte 71 generates the target ion 11. Ionophore 1215 within first electrode 1211 or within ion-selective membrane 1312 may selectively transport, or selectively bind, target ions 11 from enzyme 1415 to and within first electrode 1211 or first electrode 1311.

It will be appreciated that target analyte 71 may be any suitable analyte, enzyme 1415 may be any suitable enzyme that generates a suitable ion responsive to action upon that analyte, and ionophore 1215 may be any suitable ionophore that selectively transports and/or binds that ion generated by enzyme 1415 so as to generate an EMF based upon which the concentration of analyte 71 may be determined (whether using sensor electronics 1220 or an external device to which the sensor electronics 1220 transmits the electrophysiological signal and/or signal corresponding to ion concentration). Nonlimiting examples of analytes, enzymes, and ionophores that may be used together are listed below in Table 1.

TABLE 1

| Analyte | Enzyme | Ion generated | Ionophore |
|---|---|---|---|
| Urea | Urease | Ammonium | Nonactin |
| Glucose | Glucose | H+ (via peroxide) | Tridodecylamine, 4- |
| | oxidase | | Nonadecylpyridine, N,N- |
| | | | Dioctadecylmethylamine, |
| | | | Octadecyl isonicotinate, |
| | | | Calix[4]-aza-crown |
| Creatinine | Creatinine | Ammonium | Nonactin |
| | deaminase | | |
| Lactate | Lactate | H+ (via peroxide) | Tridodecylamine, 4- |
| | oxidase | | Nonadecylpyridine, N,N- |
| | | | Dioctadecylmethylamine, |
| | | | Octadecyl isonicotinate, |
| | | | Calix[4]-aza-crown |
| Cholesterol | Cholesterol | H+ (via peroxide) | Tridodecylamine, 4- |
| | oxidase | | Nonadecylpyridine, N,N- |
| | | | Dioctadecylmethylamine, |
| | | | Octadecyl isonicotinate, |
| | | | Calix[4]-aza-crown |
| Glutamate | Glutamate | Ammonium | Nonactin |
| | oxidase/ | | |
| | Glutamate | | |
| | dehydrogenase | | |
| Galactose | Galactose/ | H+ (via peroxide) | Tridodecylamine, 4- |
| | oxidase | | Nonadecylpyridine, N,N- |
| | | | Dioctadecylmethylamine, |
| | | | Octadecyl isonicotinate, |
| | | | Calix[4]-aza-crown |

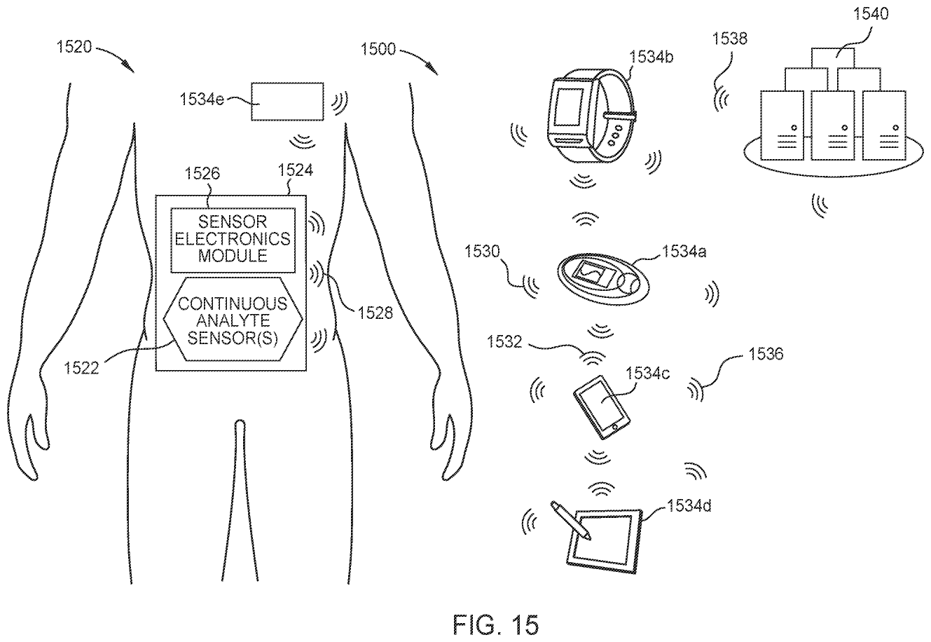

FIG. 15 is a diagram depicting an example continuous analyte monitoring system 1500 configured to measure one or more target ions and/or other analytes as discussed herein. The monitoring system 1500 includes an analyte sensor system 1524 operatively connected to a host 1520 and a plurality of display devices 1534 a-e according to certain aspects of the present disclosure. It should be noted that the display device 1534e alternatively or in addition to being a display device, may be a medicament delivery device that can act cooperatively with the analyte sensor system 1524 to deliver medicaments to host 1520. The analyte sensor system 1524 may include a sensor electronics module 1526 and a continuous analyte sensor 1522 associated with the sensor electronics module 1526. The sensor electronics module 1526 may be in direct wireless communication with one or more of the plurality of the display devices 1534a-e via wireless communications signals.

As will be discussed in greater detail below, display devices 1534a-e may also communicate amongst each other and/or through each other to analyte sensor system 1524. For ease of reference, wireless communications signals from analyte sensor system 1524 to display devices 1534a-e can be referred to as “uplink” signals 1528. Wireless communications signals from, e.g., display devices 1534a-e to analyte sensor system 1524 can be referred to as “downlink” signals 1530. Wireless communication signals between two or more of display devices 1534a-e may be referred to as “crosslink” signals 1532. Additionally, wireless communication signals can include data transmitted by one or more of display devices 1534a-d via “long-range” uplink signals 1536 (e.g., cellular signals) to one or more remote servers 1540 or network entities, such as cloud-based servers or databases, and receive long-range downlink signals 1538 transmitted by remote servers 1540.

The sensor electronics module 1526 includes sensor electronics that are configured to process sensor information and generate transformed sensor information. In certain embodiments, the sensor electronics module 1526 includes electronic circuitry associated with measuring and processing data from continuous analyte sensor 1522, including prospective algorithms associated with processing and calibration of the continuous analyte sensor data. The sensor electronics module 1526 can be integral with (non-releasably attached to) or releasably attachable to the continuous analyte sensor 1522 achieving a physical connection therebetween. The sensor electronics module 1526 may include hardware, firmware, and/or software that enables analyte level measurement. For example, the sensor electronics module 1526 can include a potentiostat, a power source for providing power to continuous analyte sensor 1522, other components useful for signal processing and data storage, and a telemetry module for transmitting data from itself to one or more display devices 1534a-e. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, each of which are incorporated herein by reference in their entirety for all purposes.

Display devices 1534a-e are configured for displaying, alarming, and/or basing medicament delivery on the sensor information that has been transmitted by the sensor electronics module 1526 (e.g., in a customized data package that is transmitted to one or more of display devices 1534a-e based on their respective preferences). Each of the display devices 1534a-e can include a display such as a touchscreen display for displaying sensor information to a user (most often host 1520 or a caretaker/medical professional) and/or receiving inputs from the user. In some embodiments, the display devices 1534a-e may include other types of user interfaces such as a voice user interface instead of or in addition to a touchscreen display for communicating sensor information to the user of the display device 1534a-e and/or receiving user inputs. In some embodiments, one, some or all of the display devices 1534a-e are configured to display or otherwise communicate the sensor information as it is communicated from the sensor electronics module 1526 (e.g., in a data package that is transmitted to respective display devices 1534a-e), without any additional prospective processing required for calibration and real-time display of the sensor information.

In the embodiment of FIG. 15, one of the plurality of display devices 1534a-e may be a custom display device 1534a specially designed for displaying certain types of displayable sensor information associated with analyte values received from the sensor electronics module 1526 (e.g., a numerical value and an arrow, in some embodiments). In some embodiments, one of the plurality of display devices 1534a-e may be a handheld device 1534c, such as a mobile phone based on the Android, iOS operating system or other operating system, a palm-top computer and the like, where handheld device 1534c may have a relatively larger display and be configured to display a graphical representation of the continuous sensor data (e.g., including current and historic data). Other display devices can include other hand-held devices, such as a tablet 1534d, a smart watch 1534b, a medicament delivery device 1534e, a blood glucose meter, and/or a desktop or laptop computer.

As discussed above, because the different display devices 1534a-e provide different user interfaces, content of the data packages (e.g., amount, format, and/or type of data to be displayed, alarms, and the like) can be customized (e.g., programmed differently by the manufacture and/or by an end user) for each particular display device and/or display device type. Accordingly, in the embodiment of FIG. 12A, one or more of display devices 1534a-e can be in direct or indirect wireless communication with the sensor electronics module 1526 to enable a plurality of different types and/or levels of display and/or functionality associated with the sensor information, which is described in more detail elsewhere herein.

### Additional Considerations

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, acc, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof, adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. A monitoring system, comprising:
a continuous analyte sensor configured to generate analyte measurements associated with analyte levels of a patient; and
a sensor electronics module coupled to the continuous analyte sensor and configured to receive and process the analyte measurements.

2. The monitoring system of claim 1, wherein the continuous analyte sensor comprises:
a substrate,
a working electrode disposed on the substrate,
a reference electrode disposed on the substrate, wherein the analyte measurements generated by the continuous analyte sensor correspond to an electromotive force at least in part based on a potential difference generated between the working electrode and the reference electrode.

3. The monitoring system of claim 1, wherein:
the continuous analyte sensor comprises a continuous potassium sensor, and
the analyte measurements include potassium measurements.

4. The monitoring system of claim 3, further comprising:
a memory comprising executable instructions; and
one or more processors in data communication with the memory and configured to execute the executable instructions to:
receive potassium data associated with the potassium measurements from the sensor electronics module;
process the potassium data to determine at least one potassium trend based on the potassium data; and
generate a kidney disease prediction based on the at least one potassium trend for the patient.

5. The monitoring system of claim 4, wherein the kidney disease prediction is indicative of at least one of:
a risk of future kidney disease in the patient;
a current presence of kidney disease in the patient;
a severity of kidney disease in the patient; or
a level of improvement or deterioration of the kidney disease in the patient.

6. The monitoring system of claim 5, wherein the severity of kidney disease corresponds to a stage of chronic kidney disease.

7. The monitoring system of claim 4, further comprising generating one or more recommendations for treatment or prevention of kidney disease based, at least in part, on the kidney disease prediction.

8. The monitoring system of claim 7, wherein the one or more recommendations comprise at least one of:
a lifestyle modification recommendation;
a medication recommendation;
an intervention recommendation; or
a recommendation to seek additional diagnostic testing.

9. The monitoring system of claim 7, wherein the one or more recommendations comprise a recommendation to administer a kidney function challenge test.

10. The monitoring system of claim 7, wherein the one or more recommendations comprise an alert or alarm indicating at least one of:
an abnormal analyte level;
an abnormal analyte rate of change;
an abnormal analyte clearance rate; or
an abnormal analyte variance.

11. The monitoring system of claim 4, wherein:
the continuous analyte sensor further comprises a continuous glucose sensor,
the analyte measurements further include glucose measurements,
the processor is further configured to receive glucose data associated with the glucose measurements from the sensor electronics module, and
the kidney disease prediction is further based on the glucose data.

12. The monitoring system of claim 4, further comprising:
one or more non-analyte sensors, wherein the processor is further configured to:
receive non-analyte sensor data generated for the patient using the one or more non-analyte sensors, wherein the kidney disease prediction is generated based on the non-analyte sensor data.

13. The monitoring system of claim 12, wherein the one or more non-analyte sensors comprise at least one of: an insulin pump, an accelerometer, a temperature sensor, an electrocardiogram (ECG) sensor, a heart rate monitor, a blood pressure sensor, an impedance, or a respiratory sensor.

14. The monitoring system of claim 4, wherein the kidney disease prediction is generated using a model trained based on population data including records of historical patients with varying stages of kidney disease.

15. The monitoring system of claim 4, wherein the processor is further configured to:
obtain at least one of demographic information, food consumption information, activity level information, medication information, health and sickness information, disease information, or kidney disease stage information related to the patient; and
wherein the kidney disease prediction is generated based on at least one of the food consumption information, the activity level information, the medication information, the health and sickness information, disease information, or the kidney disease stage information related to the patient.
